(19) United States
(12) Patent Application Publication
(10) Pub. No.: US 2017/0348376 A1
(43) Pub. Date: Dec. 7, 2017

(54) TUBULYSIN CONJUGATE FOR USE IN TREATING CANCER

(71) Applicant: ENDOCYTE, INC., West Lafayette, IN (US)

(72) Inventors: Christopher Paul LEAMON, West Lafayette, IN (US); Joseph Anand REDDY, West Lafayette, IN (US); Binh NGUYEN, Indianapolis, IN (US); Alicia BLOOMFIELD, Greenfield, IN (US); Melissa NELSON, Delphi, IN (US); Ryan DORTON, Lafayette, IN (US)

(21) Appl. No.: 15/610,855

(22) Filed: Jun. 1, 2017

Related U.S. Application Data
(60) Provisional application No. 62/345,409, filed on Jun. 3, 2016.

(51) Int. Cl. A61K 38/07; A61K 9/00; A61K 47/65; A61K 51/08; A61K 31/519

(52) U.S. Cl. CPC A61K 38/07; A61K 51/088; A61K 31/519; A61K 47/65; A61K 9/0019; A61K 9/0024

(57) ABSTRACT

The invention described herein pertains to drug delivery conjugates for targeted therapy. The invention described herein relates to methods of treating folate receptor-expressing cancers with a folate-tubulysin conjugate. The invention described herein also relates to methods of treating folate-expressing cancers with a folate tubulysin conjugate in patients where stable disease results after treatment with the folate-tubulysin conjugate.

### CROSS-REFERENCE TO RELATED APPLICATION

This application claims priority under 35 U.S.C. §119 to U.S. Provisional Application Ser. No. 62/345,409 entitled “TUBULYSIN CONJUGATE FOR USE IN TREATING CANCER,” which was filed on Jun. 3, 2016 and is expressly incorporated herein by reference.

### FIELD OF THE INVENTION

The invention described herein pertains to drug delivery conjugates for targeted therapy. The invention described herein relates to methods of treating folate receptor-expressing cancers with a folate-tubulysin conjugate. The invention described herein also relates to methods of treating folate-expressing cancers with a folate tubulysin conjugate in patients where stable disease results after treatment with the folate-tubulysin conjugate.

### BACKGROUND OF THE INVENTION

Folate plays important roles in nucleotide biosynthesis and cell division, intracellular activities which occur in both malignant and certain normal cells. The folate receptor has a high affinity for folate, which, upon binding the folate receptor, impacts the cell cycle in dividing cells. As a result, folate receptors have been implicated in a variety of cancers (e.g., ovarian, endometrial, lung and breast) which have been shown to demonstrate high folate receptor expression. In contrast, folate receptor expression in normal tissues is limited (e.g., kidney, liver, intestines and placenta). This differential expression of the folate receptor in neoplastic and normal tissues makes the folate receptor an ideal target for small molecule drug development. The development of folate conjugates represents one avenue for the discovery of new treatments that take advantage of differential expression of the folate receptor. There is a great need for the development of folate conjugates, methods to identify folate receptor positive cancers, and methods to treat patients with folate receptor positive cancers.

It has been determined that Compound I

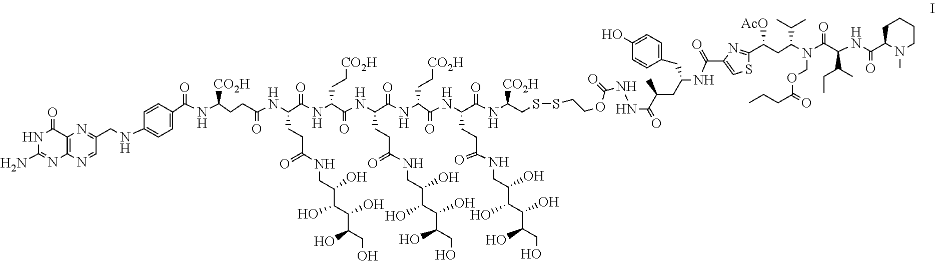 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c2ccc(NCc3cnc4nc(N)[nH]c(=O)c4n3)cc2)C(=O)O)C(=O)O)cs1)C(C)C]

or a pharmaceutically acceptable salt thereof, is useful for the treatment of cancer.

### SUMMARY OF THE INVENTION

In some embodiments a method is provided for treating a cancer in a patient in need of such treatment comprising, administering to the patient a therapeutically effective amount of a compound of the formula I

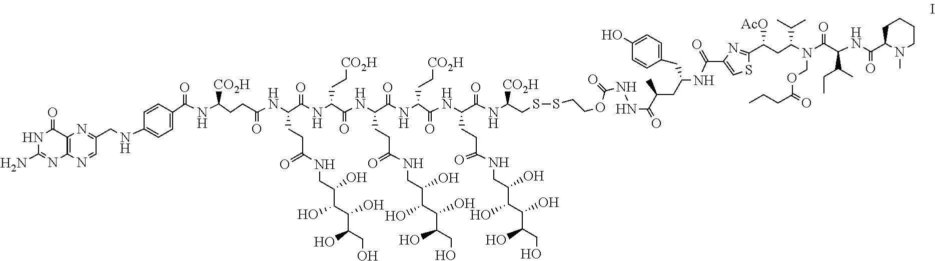 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c2ccc(NCc3cnc4nc(N)[nH]c(=O)c4n3)cc2)C(=O)O)C(=O)O)cs1)C(C)C]

or a pharmaceutically acceptable salt thereof. In some aspects of these embodiments, the cancer is a folate receptor-expressing cancer. In some aspects of these embodiments, the compound is at least about 98 percent pure. In some aspects of these embodiments, the cancer is selected from the group consisting of a carcinoma, a sarcoma, a lymphoma, a melanoma, a mesothelioma, a nasopharyngeal carcinoma, a leukemia, an adenocarcinoma, and a myeloma.

In some aspects of these embodiments, the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, small cell lung cancer, cancer of the adrenal gland, sarcoma of soft tissue, cancer of the urethra, cancer of the penis, prostate cancer, chronic leukemia, acute leukemia, lymphocytic lymphoma, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, cholangiocarcinoma, Hurthle cell thyroid cancer, cancer of the head and neck, malignant neoplasm of parotid gland, appendiceal cancer, duodenal cancer, laryngeal, metastatic hepatocellular carcinoma, metastatic squamous cell left gingiva, leiomyosarcoma and adenocarcinoma of the gastroesophageal junction. In some aspects of these embodiments, the cancer is selected from the group consisting of triple-negative breast cancer, pleural mesothelioma, non-small cell lung cancer, small cell lung cancer, adenocarcinoma of the gastroesophageal junction, ovarian cancer, cancer of the head and neck, malignant neoplasm of parotid gland, appendiceal cancer, duodenal cancer, laryngeal, metastatic hepatocellular carcinoma, metastatic squamous cell left gingiva, leiomyosarcoma and endometrial cancer. In some aspects of these embodiments, the cancer is triple-negative breast cancer. In some aspects of these embodiments, the cancer is non-small cell lung cancer. In some aspects of these embodiments, the cancer is small cell lung cancer. In some aspects of these embodiments, the cancer is adenocarcinoma of the gastroesophageal junction. In some aspects of these embodiments, the cancer is ovarian cancer. In some aspects of these embodiments, the cancer is endometrial cancer. In some aspects of these embodiments, the cancer is pleural mesothelioma. In some aspects of these embodiments, the cancer is leiomyosarcoma. In some aspects of these embodiments, the compound of formula I, or a pharmaceutically acceptable salt thereof, is administered in a parenteral dosage form.

In some aspects of these embodiments, the parenteral dosage form is selected from the group consisting of intradermal, subcutaneous, intramuscular, intraperitoneal, intravenous, and intrathecal. In some aspects of these embodiments, the therapeutically effective amount is from about 0.5 mg/m² to about 100.0 mg/m². In some aspects of these embodiments, the therapeutically effective amount is from about 0.5 mg/m² to about 90.0 mg/m². In some aspects of these embodiments, the therapeutically effective amount is from about 0.5 mg/m² to about 80.0 mg/m². In some aspects of these embodiments, the therapeutically effective amount is from about 0.5 mg/m² to about 70.0 mg/m². In some aspects of these embodiments, the therapeutically effective amount is from about 0.5 mg/m² to about 60.0 mg/m². In some aspects of these embodiments, the therapeutically effective amount is from about 0.5 mg/m² to about 50.0 mg/m². In some aspects of these embodiments, the therapeutically effective amount is from about 0.5 mg/m² to about 40.0 mg/m². In some aspects of these embodiments, the therapeutically effective amount is from about 0.5 mg/m² to about 35.0 mg/m². In some aspects of these embodiments, the therapeutically effective amount is from about 0.5 mg/m² to about 30.0 mg/m². In some aspects of these embodiments, the therapeutically effective amount is from about 0.5 mg/m² to about 25.0 mg/m². In some aspects of these embodiments, the therapeutically effective amount is from about 0.5 mg/m² to about 24.5 mg/m². In some aspects of these embodiments, the therapeutically effective amount is from about 0.5 mg/m² to about 24.0 mg/m². In some aspects of these embodiments, the therapeutically effective amount is from about 0.5 mg/m² to about 23.5 mg/m². In some aspects of these embodiments, the therapeutically effective amount is from about 0.5 mg/m² to about 23.0 mg/m². In some aspects of these embodiments, the therapeutically effective amount is from about 0.5 mg/m² to about 22.5 mg/m². In some aspects of these embodiments, the therapeutically effective amount is from about 0.5 mg/m² to about 22.0 mg/m². In some aspects of these embodiments, the therapeutically effective amount is from about 0.5 mg/m² to about 21.5 mg/m². In some aspects of these embodiments, the therapeutically effective amount is from about 0.5 mg/m² to about 21.0 mg/m². In some aspects of these embodiments, the therapeutically effective amount is from about 0.5 mg/m² to about 20.5 mg/m². In some aspects of these embodiments, the therapeutically effective amount is from about 0.5 mg/m² to about 20.0 mg/m². In some aspects of these embodiments, the therapeutically effective amount is from about 0.5 mg/m² to about 19.5 mg/m². In some aspects of these embodiments, the therapeutically effective amount is from about 0.5 mg/m² to about 19.0 mg/m². In some aspects of these embodiments, the therapeutically effective amount is from about 0.5 mg/m² to about 18.5 mg/m². In some aspects of these embodiments, the therapeutically effective amount is from about 0.5 mg/m² to about 18.0 mg/m². In some aspects of these embodiments, the therapeutically effective amount is from about 0.5 mg/m² to about 17.5 mg/m². In some aspects of these embodiments, the therapeutically effective amount is from about 0.5 mg/m² to about 17.0 mg/m². In some aspects of these embodiments, the therapeutically effective amount is from about 0.5 mg/m² to about 16.5 mg/m². In some aspects of these embodiments, the therapeutically effective amount is from about 0.5 mg/m² to about 16.0 mg/m². In some aspects of these embodiments, the therapeutically effective amount is from about 0.5 mg/m² to about 15.5 mg/m². In some aspects of these embodiments, the therapeutically effective amount is from about 0.5 mg/m² to about 15.0 mg/m². In some aspects of these embodiments, the therapeutically effective amount is from about 0.5 mg/m² to about 14.5 mg/m². In some aspects of these embodiments, the therapeutically effective amount is from about 0.5 mg/m² to about 14.0 mg/m². In some aspects of these embodiments, the therapeutically effective amount is from about 0.5 mg/m² to about 13.5 mg/m². In some aspects of these embodiments, the therapeutically effective amount is from about 0.5 mg/m² to about 13.0 mg/m².

In some aspects of these embodiments, the therapeutically effective amount is from about 0.5 mg/m² to about 12.5 mg/m². In some aspects of these embodiments, the therapeutically effective amount is from about 0.5 mg/m² to about 12.0 mg/m². In some aspects of these embodiments, the therapeutically effective amount is from about 0.5 mg/m² to about 11.5 mg/m². In some aspects of these embodiments, the therapeutically effective amount is from about 0.5 mg/m² to about 11.0 mg/m². In some aspects of these embodiments, the therapeutically effective amount is from about 0.5 mg/m² to about 10.5 mg/m². In some aspects of these embodiments, the therapeutically effective amount is from about 0.5 mg/m² to about 10.0 mg/m². In some aspects of these embodiments, the therapeutically effective amount is from about 0.5 mg/m² to about 9.5 mg/m². In some aspects of these embodiments, the therapeutically effective amount is from about 0.5 mg/m² to about 9.0 mg/m². In some aspects of these embodiments, the therapeutically effective amount is from about 0.5 mg/m² to about 8.5 mg/m². In some aspects of these embodiments, the therapeutically effective amount is from about 0.5 mg/m² to about 8.0 mg/m². In some aspects of these embodiments, the therapeutically effective amount is from about 0.5 mg/m² to about 7.5 mg/m². In some aspects of these embodiments, the therapeutically effective amount is from about 0.5 mg/m² to about 7.0 mg/m². In some aspects of these embodiments, the therapeutically effective amount is from about 0.5 mg/m² to about 6.5 mg/m². In some aspects of these embodiments, the therapeutically effective amount is from about 0.5 mg/m² to about 6.0 mg/m². In some aspects of these embodiments, the therapeutically effective amount is from about 0.5 mg/m² to about 5.5 mg/m². In some aspects of these embodiments, the therapeutically effective amount is from about 0.5 mg/m² to about 5.0 mg/m². In some aspects of these embodiments, the therapeutically effective amount is from about 0.5 mg/m² to about 4.5 mg/m². In some aspects of these embodiments, the therapeutically effective amount is from about 0.5 mg/m² to about 4.0 mg/m². In some aspects of these embodiments, the therapeutically effective amount is from about 0.5 mg/m² to about 3.5 mg/m². In some aspects of these embodiments, the therapeutically effective amount is from about 0.5 mg/m² to about 3.0 mg/m². In some aspects of these embodiments, the therapeutically effective amount is from about 0.5 mg/m² to about 2.5 mg/m². In some aspects of these embodiments, the therapeutically effective amount is from about 0.5 mg/m² to about 2.0 mg/m².

In other embodiments, the methods described herein further comprise detecting folate receptor overexpression by the cancer. In some aspects of these embodiments, the step of detecting occurs before the step of administering. In some aspects of these embodiments, the detecting is performed by imaging wherein the imaging is selected from the group consisting of SPECT imaging, PET imaging, IHC, and FISH. In some aspects of these embodiments, the detecting is performed by SPECT imaging.

In some aspects of these embodiments, the step of detecting comprises administering to the patient a conjugate of the formula II

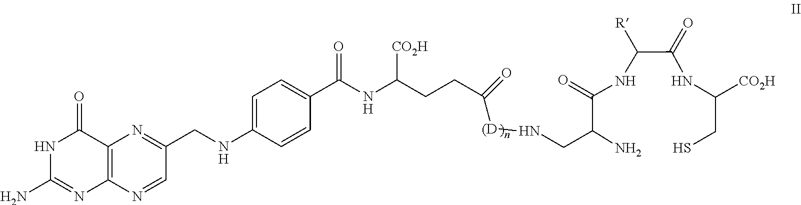

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein a radionuclide is bound to the conjugate.

In some aspects of these embodiments, the step of detecting comprises administering a conjugate of the formula III

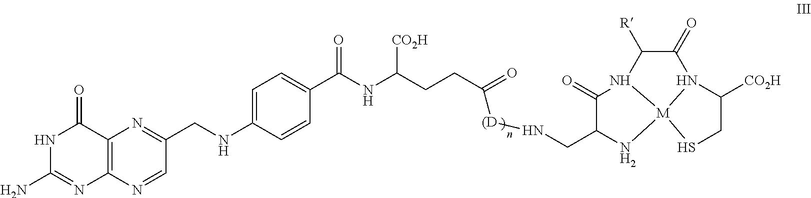

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein M is a cation of a radionuclide. In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the conjugate, or a pharmaceutically acceptable salt thereof, is an isotope of technetium.

In some aspects of these embodiments, the conjugate is of the formula

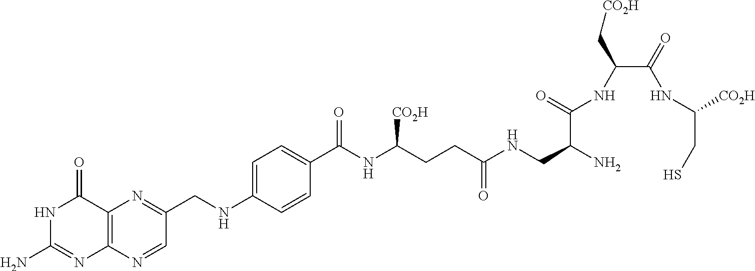 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof, wherein a radionuclide is bound to the conjugate.

In some aspects of these embodiments, the conjugate is of the formula

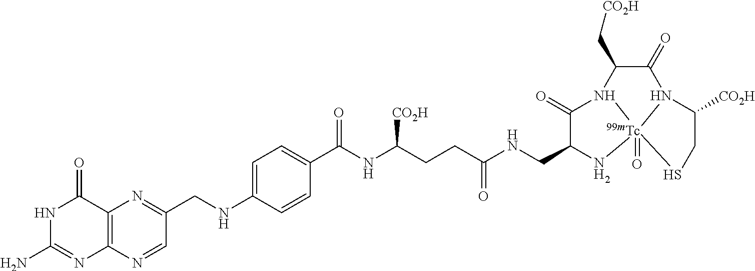

or a pharmaceutically acceptable salt thereof. This conjugate can be denoted as ^{99m}Tc-etarfolatide or pteroyl-γ-D-glutamyl-β-L-2,3-diaminopropionyl-L-asprtyl-L-cysteine complexed to ^{99m}Tc or ^{99m}Tc EC20.

In other embodiments, the methods described herein further comprise determining the folate receptor status of the patient by imaging. In some aspects of these embodiments, the imaging is SPECT imaging. In some aspects of these embodiments, the folate receptor status is based on a measurement of the percentage of evaluable lesions in the patient that are folate receptor positive. In some aspects of these embodiments, the folate receptor status of the patient correlates with a clinical benefit to the patient. In some aspects of these embodiments, the clinical benefit is selected from the group consisting of inhibition of tumor growth, stable disease, a partial response, and a complete response. In some aspects of these embodiments, the clinical benefit is stable disease. In some aspects of these embodiments, the folate receptor positive lesions indicate functionally active folate receptors.

In some aspects of these embodiments, the step of determining comprises administering to the patient a conjugate of the formula II

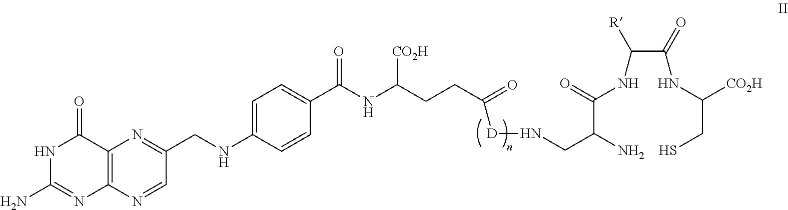

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein the conjugate is bound to a radionuclide.

In some aspects of these embodiments, the step of determining comprises administering a conjugate of the formula III

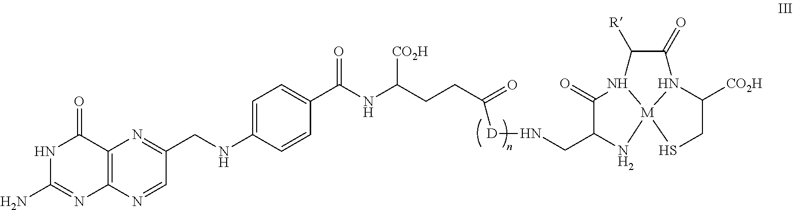

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein M is a cation of a radionuclide.

In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the conjugate, or a pharmaceutically acceptable salt thereof, is an isotope of technetium. In some aspects of these embodiments, the conjugate is of the formula

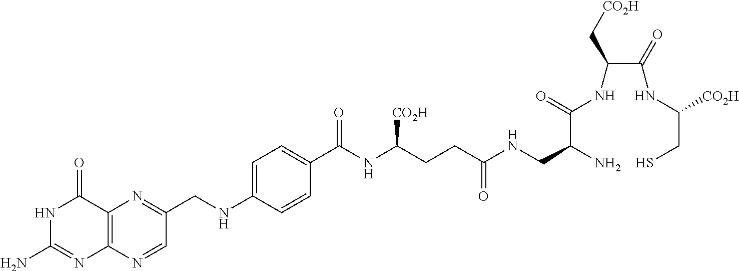 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof, wherein a radionuclide is bound to the conjugate.

In some aspects of these embodiments, the conjugate is of the formula

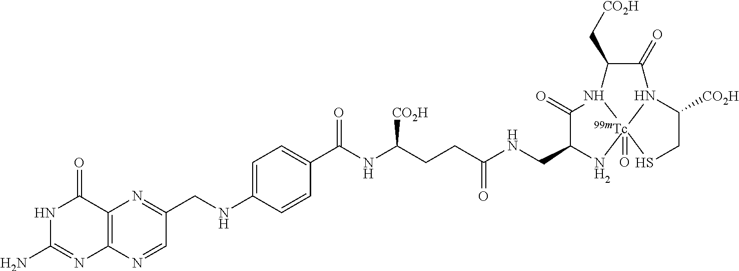

or a pharmaceutically acceptable salt thereof.

In other embodiments described herein, methods of treating a cancer in a patient in need of such treatment are provided comprising, administering to the patient a therapeutically effective amount of a compound of the formula I

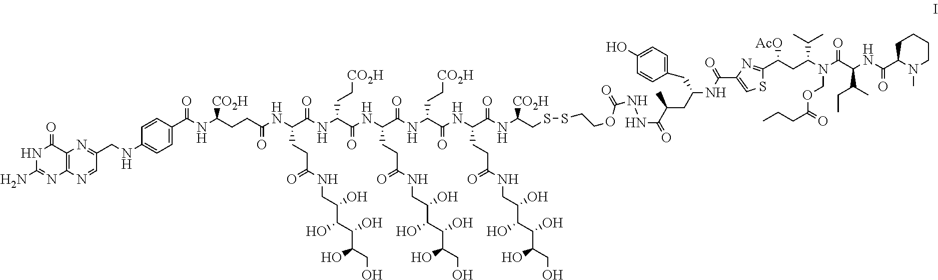 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c2ccc(NCc3cnc4nc(N)[nH]c(=O)c4n3)cc2)C(=O)O)C(=O)O)cs1)C(C)C]

or a pharmaceutically acceptable salt thereof, wherein stable disease results after the compound of formula I, or a pharmaceutically acceptable salt thereof, is administered. In some aspects of these embodiments, the patient has been treated with at least one prior treatment. In some aspects of these embodiments, the at least one prior treatment is selected from the group consisting of a chemotherapeutic agent, surgery, radiation therapy, immunotherapy, photodynamic therapy, stem cell therapy, and hyperthermia. In some aspects of these embodiments, the at least one prior treatment is a systemic treatment. In some aspects of these embodiments, the systemic treatment is selected from the group consisting of palifosfamide, 5-fluorouracil, capecitabine, pemetrexed, cisplatin, carboplatin, gemcitabine, paclitaxel, vinorelbine, eribulin, docetaxel, cyclophosphamide, doxorubicin, regorafinib, and combinations thereof. In some aspects of these embodiments, the cancer is a folate receptor-expressing cancer. In some aspects of these embodiments, the compound is at least about 98 percent pure.

In some aspects of these embodiments, the cancer is selected from the group consisting of a carcinoma, a sarcoma, a lymphoma, a melanoma, a mesothelioma, a nasopharyngeal carcinoma, a leukemia, an adenocarcinoma, and a myeloma. In some aspects of these embodiments, the cancer is selected from the group consisting of lung cancer, bone cancer, pancreatic cancer, skin cancer, cancer of the head, cancer of the neck, cutaneous melanoma, intraocular melanoma uterine cancer, ovarian cancer, leiomyosarcoma, endometrial cancer, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, cholangiocarcinoma, cancer of the head and neck, malignant neoplasm of parotid gland, appendiceal cancer, duodenal cancer, laryngeal, metastatic hepatocellular carcinoma, metastatic squamous cell left gingiva, Hurthle cell thyroid cancer, and adenocarcinoma of the gastroesophageal junction.

In some aspects of these embodiments, the cancer is selected from the group consisting of triple-negative breast cancer, pleural mesothelioma, non-small cell lung cancer, small cell lung cancer, adenocarcinoma of the gastroesophageal junction, ovarian cancer, cancer of the head and neck, malignant neoplasm of parotid gland, appendiceal cancer, duodenal cancer, laryngeal, metastatic hepatocellular carcinoma, metastatic squamous cell left gingiva, leiomyosarcoma and endometrial cancer.

In some aspects of these embodiments, the cancer is triple-negative breast cancer. In some aspects of these embodiments, the cancer is non-small cell lung cancer. In some aspects of these embodiments, the cancer is small cell lung cancer. In some aspects of these embodiments, the cancer is adenocarcinoma of the gastroesophageal junction. In some aspects of these embodiments, the cancer is ovarian cancer. In some aspects of these embodiments, the cancer is endometrial cancer. In some aspects of these embodiments, the cancer is pleural mesothelioma. In some aspects of these embodiments, the cancer is leiomyosarcoma.

In some aspects of these embodiments, the compound of formula I, or a pharmaceutically acceptable salt thereof, is administered in a parenteral dosage form. In some aspects of these embodiments, the parenteral dosage form is selected from the group consisting of intradermal, subcutaneous, intramuscular, intraperitoneal, intravenous, and intrathecal. In some aspects of these embodiments, the therapeutically effective amount is from about 0.5 mg/m² to about 100.0 mg/m². In some aspects of these embodiments, the therapeutically effective amount is from about 0.5 mg/m² to about 90.0 mg/m². In some aspects of these embodiments, the therapeutically effective amount is from about 0.5 mg/m² to about 80.0 mg/m². In some aspects of these embodiments, the therapeutically effective amount is from about 0.5 mg/m² to about 70.0 mg/m². In some aspects of these embodiments, the therapeutically effective amount is from about 0.5 mg/m² to about 60.0 mg/m². In some aspects of these embodiments, the therapeutically effective amount is from about 0.5 mg/m² to about 50.0 mg/m². In some aspects of these embodiments, the therapeutically effective amount is from about 0.5 mg/m² to about 40.0 mg/m². In some aspects of these embodiments, the therapeutically effective amount is from about 0.5 mg/m² to about 35.0 mg/m². In some aspects of these embodiments, the therapeutically effective amount is from about 0.5 mg/m² to about 30.0 mg/m². In some aspects of these embodiments, the therapeutically effective amount is from about 0.5 mg/m² to about 25.0 mg/m². In some aspects of these embodiments, the therapeutically effective amount is from about 0.5 mg/m² to about 24.5 mg/m². In some aspects of these embodiments, the therapeutically effective amount is from about 0.5 mg/m² to about 24.0 mg/m². In some aspects of these embodiments, the therapeutically effective amount is from about 0.5 mg/m² to about 23.5 mg/m². In some aspects of these embodiments, the therapeutically effective amount is from about 0.5 mg/m² to about 23.0 mg/m². In some aspects of these embodiments, the therapeutically effective amount is from about 0.5 mg/m² to about 22.5 mg/m². In some aspects of these embodiments, the therapeutically effective amount is from about 0.5 mg/m² to about 22.0 mg/m². In some aspects of these embodiments, the therapeutically effective amount is from about 0.5 mg/m² to about 21.5 mg/m². In some aspects of these embodiments, the therapeutically effective amount is from about 0.5 mg/m² to about 21.0 mg/m². In some aspects of these embodiments, the therapeutically effective amount is from about 0.5 mg/m² to about 20.5 mg/m². In some aspects of these embodiments, the therapeutically effective amount is from about 0.5 mg/m² to about 20.0 mg/m². In some aspects of these embodiments, the therapeutically effective amount is from about 0.5 mg/m² to about 19.5 mg/m². In some aspects of these embodiments, the therapeutically effective amount is from about 0.5 mg/m² to about 19.0 mg/m². In some aspects of these embodiments, the therapeutically effective amount is from about 0.5 mg/m² to about 18.5 mg/m². In some aspects of these embodiments, the therapeutically effective amount is from about 0.5 mg/m² to about 18.0 mg/m². In some aspects of these embodiments, the therapeutically effective amount is from about 0.5 mg/m² to about 17.5 mg/m². In some aspects of these embodiments, the therapeutically effective amount is from about 0.5 mg/m² to about 17.0 mg/m². In some aspects of these embodiments, the therapeutically effective amount is from about 0.5 mg/m² to about 16.5 mg/m². In some aspects of these embodiments, the therapeutically effective amount is from about 0.5 mg/m² to about 16.0 mg/m². In some aspects of these embodiments, the therapeutically effective amount is from about 0.5 mg/m² to about 15.5 mg/m². In some aspects of these embodiments, the therapeutically effective amount is from about 0.5 mg/m² to about 15.0 mg/m². In some aspects of these embodiments, the therapeutically effective amount is from about 0.5 mg/m² to about 14.5 mg/m². In some aspects of these embodiments, the therapeutically effective amount is from about 0.5 mg/m² to about 14.0 mg/m². In some aspects of these embodiments, the therapeutically effective amount is from about 0.5 mg/m² to about 13.5 mg/m². In some aspects of these embodiments, the therapeutically effective amount is from about 0.5 mg/m² to about 13.0 mg/m². In some aspects of these embodiments, the therapeutically effective amount is from about 0.5 mg/m² to about 12.5 mg/m². In some aspects of these embodiments, the therapeutically effective amount is from about 0.5 mg/m² to about 12.0 mg/m². In some aspects of these embodiments, the therapeutically effective amount is from about 0.5 mg/m² to about 11.5 mg/m². In some aspects of these embodiments, the therapeutically effective amount is from about 0.5 mg/m² to about 11.0 mg/m². In some aspects of these embodiments, the therapeutically effective amount is from about 0.5 mg/m² to about 10.5 mg/m².

In some aspects of these embodiments, the therapeutically effective amount is from about 0.5 mg/m² to about 10.0 mg/m². In some aspects of these embodiments, the therapeutically effective amount is from about 0.5 mg/m² to about 9.5 mg/m². In some aspects of these embodiments, the therapeutically effective amount is from about 0.5 mg/m² to about 9.0 mg/m². In some aspects of these embodiments, the therapeutically effective amount is from about 0.5 mg/m² to about 8.5 mg/m². In some aspects of these embodiments, the therapeutically effective amount is from about 0.5 mg/m² to about 8.0 mg/m². In some aspects of these embodiments, the therapeutically effective amount is from about 0.5 mg/m² to about 7.5 mg/m². In some aspects of these embodiments, the therapeutically effective amount is from about 0.5 mg/m² to about 7.0 mg/m². In some aspects of these embodiments, the therapeutically effective amount is from about 0.5 mg/m² to about 6.5 mg/m². In some aspects of these embodiments, the therapeutically effective amount is from about 0.5 mg/m² to about 6.0 mg/m². In some aspects of these embodiments, the therapeutically effective amount is from about 0.5 mg/m² to about 5.5 mg/m². In some aspects of these embodiments, the therapeutically effective amount is from about 0.5 mg/m² to about 5.0 mg/m². In some aspects of these embodiments, the therapeutically effective amount is from about 0.5 mg/m² to about 4.5 mg/m². In some aspects of these embodiments, the therapeutically effective amount is from about 0.5 mg/m² to about 4.0 mg/m². In some aspects of these embodiments, the therapeutically effective amount is from about 0.5 mg/m² to about 3.5 mg/m². In some aspects of these embodiments, the therapeutically effective amount is from about 0.5 mg/m² to about 3.0 mg/m². In some aspects of these embodiments, the therapeutically effective amount is from about 0.5 mg/m² to about 2.5 mg/m². In some aspects of these embodiments, the therapeutically effective amount is from about 0.5 mg/m² to about 2.0 mg/m².

In other embodiments the methods described herein further comprise detecting folate receptor overexpression by the cancer. In some aspects of these embodiments, the step of detecting occurs before the step of administering. In some aspects of these embodiments, the detecting is performed by imaging wherein the imaging is selected from the group consisting of SPECT imaging, PET imaging, IHC, and FISH. In some aspects of these embodiments, the detecting is performed by SPECT imaging.

In some aspects of these embodiments, the step of detecting comprises administering to the patient a conjugate of the formula II

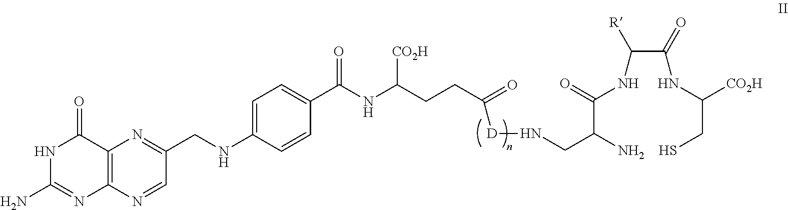

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein a radionuclide is bound to the conjugate.

In some aspects of these embodiments, the step of detecting comprises administering a conjugate of the formula III

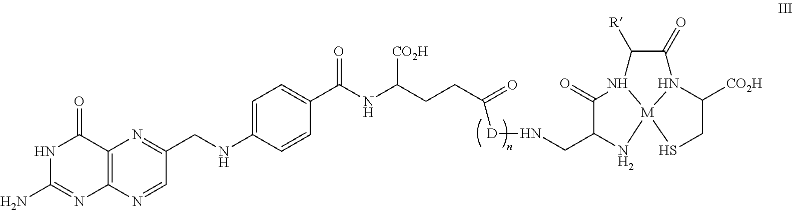

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein M is a cation of a radionuclide. In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the conjugate, or a pharmaceutically acceptable salt thereof, is an isotope of technetium.

In some aspects of these embodiments, the conjugate is of the formula

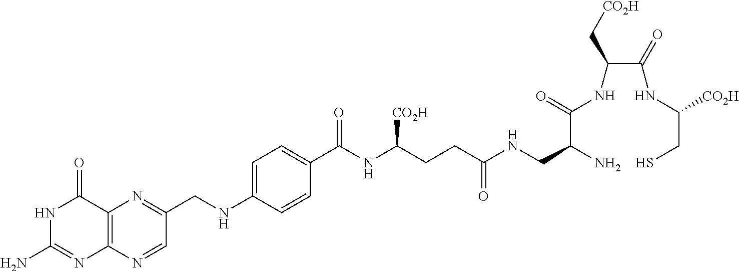 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof, and wherein a radionuclide is bound to the conjugate. In some aspects of these embodiments, the conjugate is of the formula

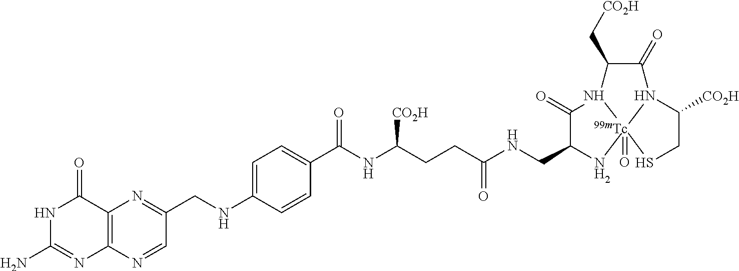

or a pharmaceutically acceptable salt thereof.

In other embodiments the methods described herein further comprise determining the folate receptor status of the patient by imaging. In some aspects of these embodiments, the imaging is SPECT imaging. In some aspects of these embodiments, the folate receptor status is based on a measurement of the percentage of evaluable lesions in the patient that are folate receptor positive. In some aspects of these embodiments, the folate receptor status of the patient correlates with a clinical benefit to the patient. In some aspects of these embodiments, the clinical benefit is selected from the group consisting of inhibition of tumor growth, stable disease, a partial response, and a complete response. In some aspects of these embodiments, the clinical benefit is stable disease.

In some aspects of these embodiments, the step of determining comprises administering to the patient a conjugate of the formula II

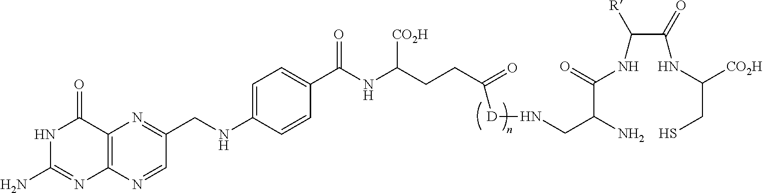

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein a radionuclide is bound to the conjugate.

In some aspects of these embodiments, the step of determining comprises administering a conjugate of the formula III

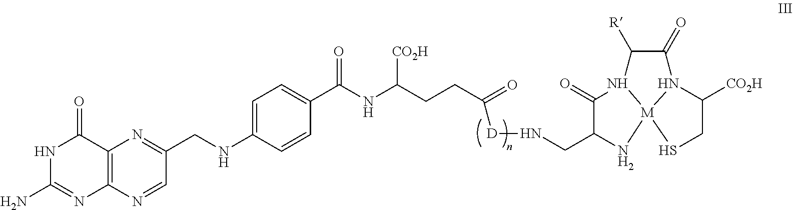

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherin M is a cation of a radionuclide.

In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the conjugate, or a pharmaceutically acceptable salt thereof, is an isotope of technetium. In some aspects of these embodiments, the conjugate is of the formula

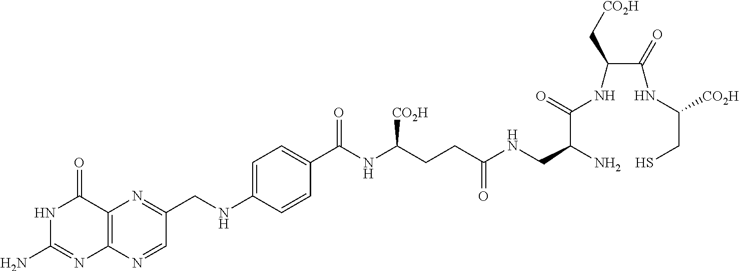 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof, wherein a radionuclide is bound to the conjugate.

In some aspects of these embodiments, the conjugate is of the formula

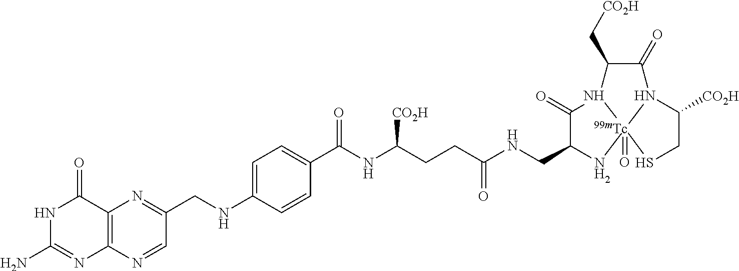

or a pharmaceutically acceptable salt thereof. In some aspects of these embodiments, unlabeled folic acid, or a pharmaceutically acceptable salt thereof, is administered to the patient before the conjugate, or a pharmaceutically acceptable salt thereof, is administered to the patient.

In some embodiments described herein are methods of treating a folate receptor-expressing cancer in a patient in need of such treatment comprising, administering to the patient a therapeutically effective amount of a compound of the formula I

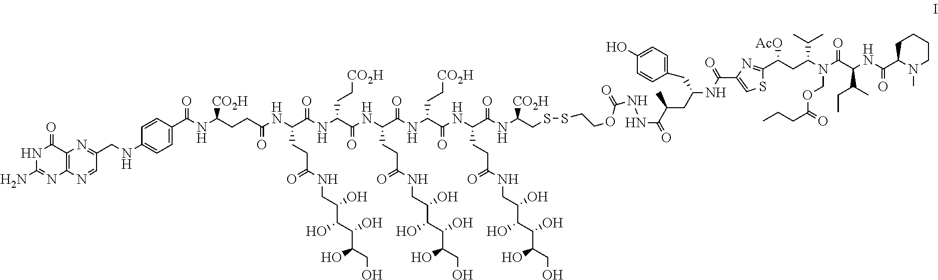 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c2ccc(NCc3cnc4nc(N)[nH]c(=O)c4n3)cc2)C(=O)O)C(=O)O)cs1)C(C)C]

or a pharmaceutically acceptable salt thereof, wherein the patient has been identified as having a folate receptor-expressing cancer.

In some aspects of these embodiments, the patient has been treated with at least one prior treatment. In some aspects of these embodiments, the at least one prior treatment is selected from the group consisting of a chemotherapeutic agent, surgery, radiation therapy, immunotherapy, photodynamic therapy, stem cell therapy, and hyperthermia. In some aspects of these embodiments, the at least one prior treatment is a systemic treatment. In some aspects of these embodiments, the systemic treatment is selected from the group consisting of palifosfamide, 5-fluorouracil, capecitabine, pemetrexed, cisplatin, carboplatin, gemcitabine, paclitaxel, vinorelbine, eribulin, docetaxel, cyclophosphamide, doxorubicin, regorafinib, and combinations thereof. In some aspects of these embodiments, the cancer is a folate receptor-expressing cancer. In some aspects of these embodiments, the compound is at least about 98 percent pure.

In some aspects of these embodiments, the cancer is selected from the group consisting of a carcinoma, a sarcoma, a lymphoma, a melanoma, a mesothelioma, a nasopharyngeal carcinoma, a leukemia, an adenocarcinoma, and a myeloma. In some aspects of these embodiments, the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, leiomyosarcoma, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, cholangiocarcinoma, cancer of the head and neck, malignant neoplasm of parotid gland, appendiceal cancer, duodenal cancer, laryngeal, metastatic hepatocellular carcinoma, metastatic squamous cell left gingiva, Hurthle cell thyroid cancer, and adenocarcinoma of the gastroesophageal junction.

In some aspects of these embodiments, the cancer is selected from the group consisting of triple-negative breast cancer, pleural mesothelioma, non-small cell lung cancer, adenocarcinoma of the gastroesophageal junction, ovarian cancer, cancer of the head and neck, malignant neoplasm of parotid gland, appendiceal cancer, duodenal cancer, laryngeal, metastatic hepatocellular carcinoma, metastatic squamous cell left gingiva, leiomyosarcoma and endometrial cancer.

In some aspects of these embodiments, the cancer is triple-negative breast cancer. In some aspects of these embodiments, the cancer is non-small cell lung cancer. In some aspects of these embodiments, the cancer is small cell lung cancer. In some aspects of these embodiments, the cancer is adenocarcinoma of the gastroesophageal junction. In some aspects of these embodiments, the cancer is ovarian cancer. In some aspects of these embodiments, the cancer is endometrial cancer. In some aspects of these embodiments, the cancer is pleural mesothelioma. In some aspects of these embodiments, the cancer is leiomyosarcoma.

In some aspects of these embodiments, the compound of formula I, or a pharmaceutically acceptable salt thereof, is administered in a parenteral dosage form. In some aspects of these embodiments, the parenteral dosage form is selected from the group consisting of intradermal, subcutaneous, intramuscular, intraperitoneal, intravenous, and intrathecal. In some aspects of these embodiments, the therapeutically effective amount is from about 0.5 mg/m² to about 100.0 mg/m². In some aspects of these embodiments, the therapeutically effective amount is from about 0.5 mg/m² to about 90.0 mg/m². In some aspects of these embodiments, the therapeutically effective amount is from about 0.5 mg/m² to about 80.0 mg/m². In some aspects of these embodiments, the therapeutically effective amount is from about 0.5 mg/m² to about 70.0 mg/m². In some aspects of these embodiments, the therapeutically effective amount is from about 0.5 mg/m² to about 60.0 mg/m². In some aspects of these embodiments, the therapeutically effective amount is from about 0.5 mg/m² to about 50.0 mg/m². In some aspects of these embodiments, the therapeutically effective amount is from about 0.5 mg/m² to about 40.0 mg/m². In some aspects of these embodiments, the therapeutically effective amount is from about 0.5 mg/m² to about 35.0 mg/m². In some aspects of these embodiments, the therapeutically effective amount is from about 0.5 mg/m² to about 30.0 mg/m². In some aspects of these embodiments, the therapeutically effective amount is from about 0.5 mg/m² to about 25.0 mg/m². In some aspects of these embodiments, the therapeutically effective amount is from about 0.5 mg/m² to about 24.5 mg/m². In some aspects of these embodiments, the therapeutically effective amount is from about 0.5 mg/m² to about 24.0 mg/m². In some aspects of these embodiments, the therapeutically effective amount is from about 0.5 mg/m² to about 23.5 mg/m². In some aspects of these embodiments, the therapeutically effective amount is from about 0.5 mg/m² to about 23.0 mg/m². In some aspects of these embodiments, the therapeutically effective amount is from about 0.5 mg/m² to about 22.5 mg/m². In some aspects of these embodiments, the therapeutically effective amount is from about 0.5 mg/m² to about 22.0 mg/m². In some aspects of these embodiments, the therapeutically effective amount is from about 0.5 mg/m² to about 21.5 mg/m². In some aspects of these embodiments, the therapeutically effective amount is from about 0.5 mg/m² to about 21.0 mg/m². In some aspects of these embodiments, the therapeutically effective amount is from about 0.5 mg/m² to about 20.5 mg/m². In some aspects of these embodiments, the therapeutically effective amount is from about 0.5 mg/m² to about 20.0 mg/m². In some aspects of these embodiments, the therapeutically effective amount is from about 0.5 mg/m² to about 19.5 mg/m². In some aspects of these embodiments, the therapeutically effective amount is from about 0.5 mg/m² to about 19.0 mg/m². In some aspects of these embodiments, the therapeutically effective amount is from about 0.5 mg/m² to about 18.5 mg/m². In some aspects of these embodiments, the therapeutically effective amount is from about 0.5 mg/m² to about 18.0 mg/m². In some aspects of these embodiments, the therapeutically effective amount is from about 0.5 mg/m² to about 17.5 mg/m². In some aspects of these embodiments, the therapeutically effective amount is from about 0.5 mg/m² to about 17.0 mg/m². In some aspects of these embodiments, the therapeutically effective amount is from about 0.5 mg/m² to about 16.5 mg/m². In some aspects of these embodiments, the therapeutically effective amount is from about 0.5 mg/m² to about 16.0 mg/m². In some aspects of these embodiments, the therapeutically effective amount is from about 0.5 mg/m² to about 15.5 mg/m². In some aspects of these embodiments, the therapeutically effective amount is from about 0.5 mg/m² to about 15.0 mg/m². In some aspects of these embodiments, the therapeutically effective amount is from about 0.5 mg/m² to about 14.5 mg/m². In some aspects of these embodiments, the therapeutically effective amount is from about 0.5 mg/m² to about 14.0 mg/m². In some aspects of these embodiments, the therapeutically effective amount is from about 0.5 mg/m² to about 13.5 mg/m². In some aspects of these embodiments, the therapeutically effective amount is from about 0.5 mg/m² to about 13.0 mg/m². In some aspects of these embodiments, the therapeutically effective amount is from about 0.5 mg/m² to about 12.5 mg/m². In some aspects of these embodiments, the therapeutically effective amount is from about 0.5 mg/m² to about 12.0 mg/m². In some aspects of these embodiments, the therapeutically effective amount is from about 0.5 mg/m² to about 11.5 mg/m². In some aspects of these embodiments, the therapeutically effective amount is from about 0.5 mg/m² to about 11.0 mg/m². In some aspects of these embodiments, the therapeutically effective amount is from about 0.5 mg/m² to about 10.5 mg/m². In some aspects of these embodiments, the therapeutically effective amount is from about 0.5 mg/m² to about 10.0 mg/m². In some aspects of these embodiments, the therapeutically effective amount is from about 0.5 mg/m² to about 9.5 mg/m². In some aspects of these embodiments, the therapeutically effective amount is from about 0.5 mg/m² to about 9.0 mg/m². In some aspects of these embodiments, the therapeutically effective amount is from about 0.5 mg/m² to about 8.5 mg/m². In some aspects of these embodiments, the therapeutically effective amount is from about 0.5 mg/m² to about 8.0 mg/m². In some aspects of these embodiments, the therapeutically effective amount is from about 0.5 mg/m² to about 7.5 mg/m². In some aspects of these embodiments, the therapeutically effective amount is from about 0.5 mg/m² to about 7.0 mg/m².

In some aspects of these embodiments, the therapeutically effective amount is from about 0.5 mg/m² to about 6.5 mg/m². In some aspects of these embodiments, the therapeutically effective amount is from about 0.5 mg/m² to about 6.0 mg/m². In some aspects of these embodiments, the therapeutically effective amount is from about 0.5 mg/m² to about 5.5 mg/m². In some aspects of these embodiments, the therapeutically effective amount is from about 0.5 mg/m² to about 5.0 mg/m². In some aspects of these embodiments, the therapeutically effective amount is from about 0.5 mg/m² to about 4.5 mg/m². In some aspects of these embodiments, the therapeutically effective amount is from about 0.5 mg/m² to about 4.0 mg/m². In some aspects of these embodiments, the therapeutically effective amount is from about 0.5 mg/m² to about 3.5 mg/m². In some aspects of these embodiments, the therapeutically effective amount is from about 0.5 mg/m² to about 3.0 mg/m². In some aspects of these embodiments, the therapeutically effective amount is from about 0.5 mg/m² to about 2.5 mg/m². In some aspects of these embodiments, the therapeutically effective amount is from about 0.5 mg/m² to about 2.0 mg/m².

In other embodiments the methods described herein further comprise detecting folate receptor overexpression by the cancer. In some aspects of these embodiments, the step of detecting occurs before the step of administering. In some aspects of these embodiments, the detecting is performed by imaging and the imaging is selected from the group consisting of SPECT imaging, PET imaging, IHC, and FISH. In some aspects of these embodiments, the detecting is performed by SPECT imaging.

In some aspects of these embodiments, the step of detecting comprises administering to the patient a conjugate of the formula II

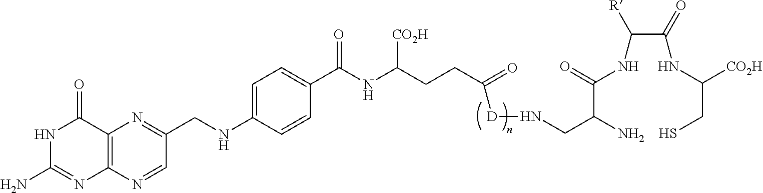

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein a radionuclide is bound to the conjugate.

In some aspects of these embodiments, the step of detecting comprises administering a conjugate of the formula III

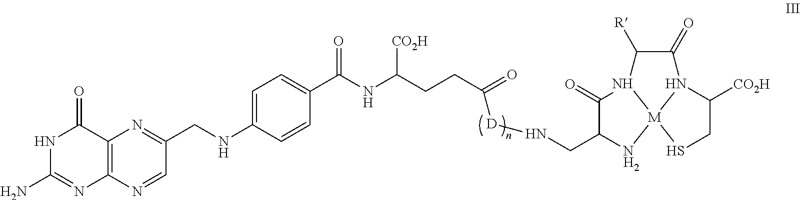

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein M is a cation of a radionuclide. In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the conjugate, or a pharmaceutically acceptable salt thereof, is an isotope of technetium.

In some aspects of these embodiments, the conjugate is of the formula

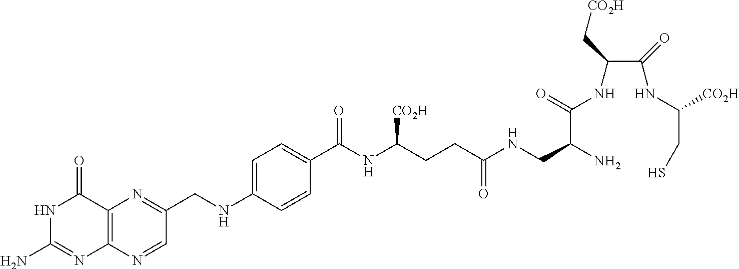 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof, wherein a radionuclide is bound to the conjugate. In some aspects of these embodiments, the conjugate is of the formula

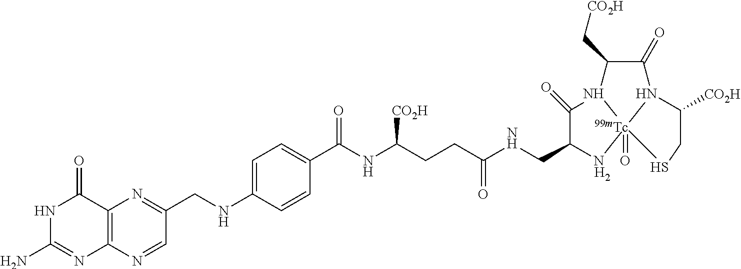

or a pharmaceutically acceptable salt thereof.

In other embodiments the methods described herein further comprise determining the folate receptor status of the patient by imaging. In some aspects of these embodiments, the imaging is SPECT imaging. In some aspects of these embodiments, the folate receptor status is based on a measurement of the percentage of evaluable lesions in the patient that are folate receptor positive. In some aspects of these embodiments, the folate receptor status of the patient correlates with a clinical benefit to the patient. In some aspects of these embodiments, the clinical benefit is selected from the group consisting of inhibition of tumor growth, stable disease, a partial response, and a complete response. In some aspects of these embodiments, the clinical benefit is stable disease.

In some aspects of these embodiments, the step of determining comprises administering to the patient a conjugate of the formula II

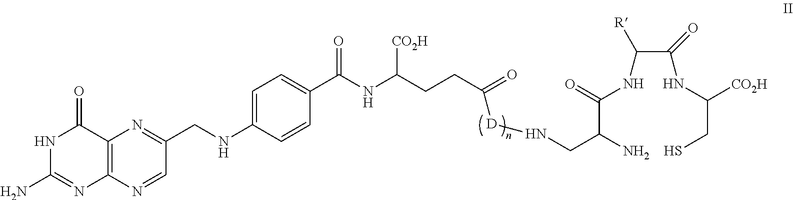

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein a radionuclide is bound to the conjugate.

In some aspects of these embodiments, the step of determining comprises administering a conjugate of the formula III

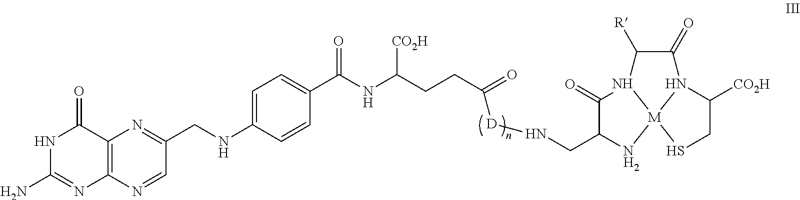

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherin M is a cation of a radionuclide.

In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the conjugate, or a pharmaceutically acceptable salt thereof, is an isotope of technetium. In some aspects of these embodiments, the conjugate is of the formula

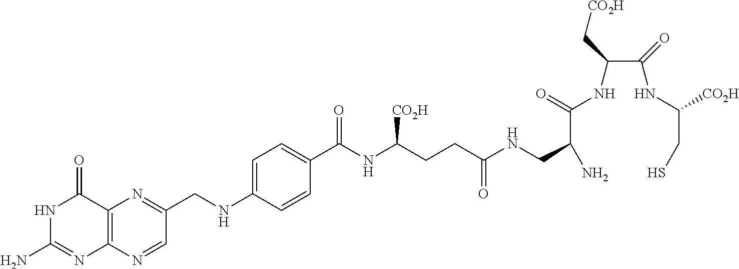 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof, and a radionuclide is bound to the conjugate.

In some aspects of these embodiments, the conjugate is of the formula

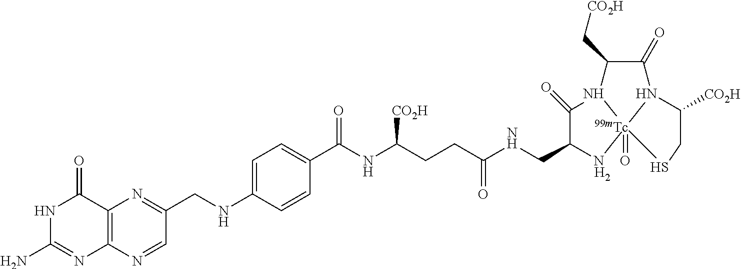

or a pharmaceutically acceptable salt thereof. In some aspects of these embodiments, unlabeled folic acid, or a pharmaceutically acceptable salt thereof, is administered to the patient before the conjugate, or a pharmaceutically acceptable salt thereof, is administered to the patient.

Embodiments of the invention are further described by the following enumerated clauses:
1. A method for treating a cancer in a patient in need of such treatment comprising, administering to the patient a therapeutically effective amount of a compound of the formula I

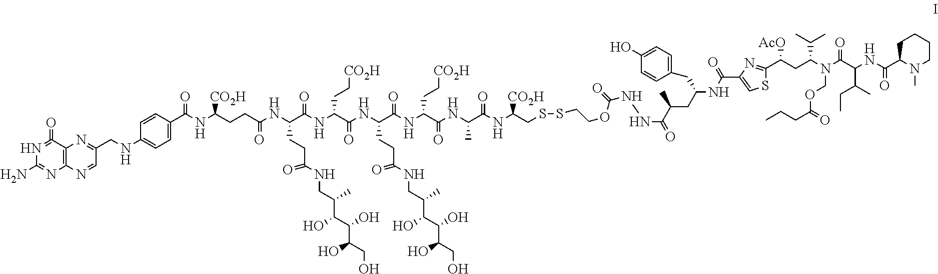

or a pharmaceutically acceptable salt thereof.
2. The method of clause 1, wherein the cancer is a folate receptor expressing cancer.
3. The method of clause 1 or 2, wherein the compound is at least about 98 percent pure.
4. The method of any one of clauses 1 to 3, wherein the cancer is selected from the group consisting of a carcinoma, a sarcoma, a lymphoma, a melanoma, a mesothelioma, a nasopharyngeal carcinoma, a leukemia, an adenocarcinoma, and a myeloma.
5. The method of any one of clauses 1 to 3, wherein the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, leiomyosarcoma, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, cholangiocarcinoma, cancer of the head and neck, malignant neoplasm of parotid gland, appendiceal cancer, duodenal cancer, laryngeal, metastatic hepatocellular carcinoma, metastatic squamous cell left gingiva, Hurthle cell thyroid cancer and adenocarcinoma of the gastroesophageal junction.
6. The method of any one of clauses 1 to 5, wherein the cancer is selected from the group consisting of triple-negative breast cancer, pleural mesothelioma, non-small cell lung cancer, small cell lung cancer, adenocarcinoma of the gastroesophageal junction, ovarian cancer, cancer of the head and neck, malignant neoplasm of parotid gland, appendiceal cancer, duodenal cancer, laryngeal, metastatic hepatocellular carcinoma, metastatic squamous cell left gingiva, leiomyosarcoma and endometrial cancer.
7. The method of any one of clauses 1 to 6, wherein the cancer is triple-negative breast cancer.
8. The method of any one of clauses 1 to 6, wherein the cancer is non-small cell lung cancer.
9. The method of any one of clauses 1 to 6, wherein the cancer is adenocarcinoma of the gastroesophageal junction.
10. The method of any one of clauses 1 to 6, wherein the cancer is ovarian cancer.
11. The method of any one of clauses 1 to 6, wherein the cancer is pleural mesothelioma.
11a. The method of any one of clauses 1 to 6, wherein the cancer is small cell lung cancer.
11b. The method of any one of clauses 1 to 6, wherein the cancer is leiomyosarcoma.
12. The method of any one of clauses 1 to 11, wherein the compound of formula I, or a pharmaceutically acceptable salt thereof, is administered in a parenteral dosage form.
13. The method of clause 12, wherein the parenteral dosage form is selected from the group consisting of intradermal, subcutaneous, intramuscular, intraperitoneal, intravenous, and intrathecal.
14. The method of any one of the preceding clauses, wherein the therapeutically effective amount is from about 0.5 mg/m² to about 100.0 mg/m², or about 0.5 mg/m² to about 90.0 mg/m², or about 0.5 mg/m² to about 80.0 mg/m², or about 0.5 mg/m² to about 70.0 mg/m², or about 0.5 mg/m² to about 60.0 mg/m², or about 0.5 mg/m² to about 50.0 mg/m², or 0.5 mg/m² to about 40.0 mg/m², or about 0.5 mg/m² to about 35.0 mg/m², or about 0.5 mg/m² to about 30.0 mg/m², or about 0.5 mg/m² to about 25.0 mg/m², or 0.5 mg/m² to about 24.5 mg/m², or about 0.5 mg/m² to about 24.0 mg/m², or about 0.5 mg/m² to about 23.5 mg/m², or about 0.5 mg/m² to about 23.0 mg/m², or about 0.5 mg/m² to about 22.5 mg/m², or about 0.5 mg/m² to about 22.0 mg/m², or about 0.5 mg/m² to about 21.5 mg/m², or about 0.5 mg/m² to about 21.0 mg/m², or about 0.5 mg/m² to about 20.0 mg/m².
15. The method of any one of clauses 1 to 14, wherein the therapeutically effective amount is from about 0.5 mg/m² to about 19.0 mg/m².
16. The method of any one of clauses 1 to 15, wherein the therapeutically effective amount is from about 0.5 mg/m² to about 18.0 mg/m².
17. The method of any one of clauses 1 to 16, wherein the therapeutically effective amount is from about 0.5 mg/m² to about 17.0 mg/m².
18. The method of any one of clauses 1 to 17, wherein the therapeutically effective amount is from about 0.5 mg/m² to about 16.0 mg/m².
19. The method of any one of clauses 1 to 18, wherein the therapeutically effective amount is from about 0.5 mg/m² to about 15.0 mg/m².
20. The method of any one of clauses 1 to 19, wherein the therapeutically effective amount is from about 0.5 mg/m² to about 14.0 mg/m².
21. The method of any one of clauses 1 to 20, wherein the therapeutically effective amount is from about 0.5 mg/m² to about 13.0 mg/m².
22. The method of any one of clauses 1 to 21, wherein the therapeutically effective amount is from about 0.5 mg/m² to about 12.0 mg/m².
23. The method of any one of clauses 1 to 22, wherein the therapeutically effective amount is from about 0.5 mg/m² to about 11.0 mg/m².
24. The method of any one of clauses 1 to 23, wherein the therapeutically effective amount is from about 0.5 mg/m² to about 10.0 mg/m².
25. The method of any one of clauses 1 to 24, wherein the therapeutically effective amount is from about 0.5 mg/m² to about 9.0 mg/m².
26. The method of any one of clauses 1 to 25, wherein the therapeutically effective amount is from about 0.5 mg/m² to about 8.0 mg/m².
27. The method of any one of clauses 1 to 26, wherein the therapeutically effective amount is from about 0.5 mg/m² to about 7.0 mg/m².
28. The method of any one of clauses 1 to 27, wherein the therapeutically effective amount is from about 0.5 mg/m² to about 6.0 mg/m².
29. The method of any one of clauses 1 to 28, wherein the therapeutically effective amount is from about 0.5 mg/m² to about 5.0 mg/m².
30. The method of any one of clauses 1 to 29, wherein the therapeutically effective amount is from about 0.5 mg/m² to about 4.0 mg/m².
31. The method of any one of clauses 1 to 30, wherein the therapeutically effective amount is from about 0.5 mg/m² to about 3.5 mg/m².
32. The method of any one of clauses 1 to 31, wherein the therapeutically effective amount is from about 0.5 mg/m² to about 3.0 mg/m².
33. The method of any one of clauses 1 to 32, wherein the therapeutically effective amount is from about 0.5 mg/m² to about 2.5 mg/m².
34. The method of any one of clauses 1 to 33, wherein the therapeutically effective amount is from about 0.5 mg/m² to about 2.0 mg/m².
35. The method of any one of clauses 1 to 34, further comprising detecting folate receptor overexpression by the cancer.
36. The method of clause 35, wherein the step of detecting occurs before the step of administering.
37. The method of clause 35 or 36, wherein the detecting is performed by imaging and wherein the imaging is selected from the group consisting of SPECT imaging, PET imaging, IHC, and FISH.
38. The method of any one of clauses 35 to 37, wherein the detecting is performed by SPECT imaging.
39. The method of any one of clauses 1 to 38, further comprising determining the folate receptor status of the patient by imaging.
40. The method of clause 39, wherein the imaging is SPECT imaging.
41. The method of clause 39 or 40, wherein the folate receptor status is based on a measurement of the percentage of evaluable lesions in the patient that are folate receptor positive.
42. The method of any one of clauses 39 to 41, wherein the folate receptor status of the patient correlates with a clinical benefit to the patient.
43. The method of clause 42, wherein the clinical benefit is selected from the group consisting of inhibition of tumor growth, stable disease, a partial response, and a complete response.
44. The method of clause 42 or 43 wherein the clinical benefit is stable disease.
45. The method of any one of clauses 41 to 44 wherein the folate receptor positive lesions indicate functionally active folate receptors.
46. The method of any one of clauses 35 to 45, wherein the step of detecting comprises administering to the patient a conjugate of the formula II

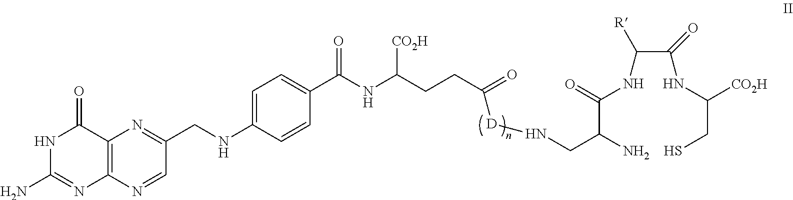

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein a radionuclide is bound to the conjugate.
47. The method of any one of clauses 45 to 46, wherein the step of detecting comprises administering a conjugate of the formula III

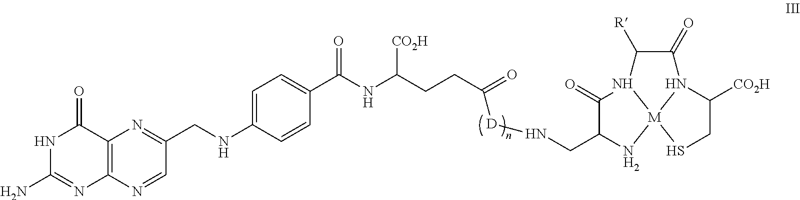

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein M is a cation of a radionuclide.
48. The method of clause 47, wherein M in the conjugate, or a pharmaceutically acceptable salt thereof, is selected from the group consisting of an isotope of gallium, an isotope of indium, an isotope of copper, an isotope of technetium, and an isotope of rhenium.
49. The method of clause 47 or 48, wherein M in the conjugate, or a pharmaceutically acceptable salt thereof, is an isotope of technetium.
50. The method of clause 46, wherein the conjugate is of the formula

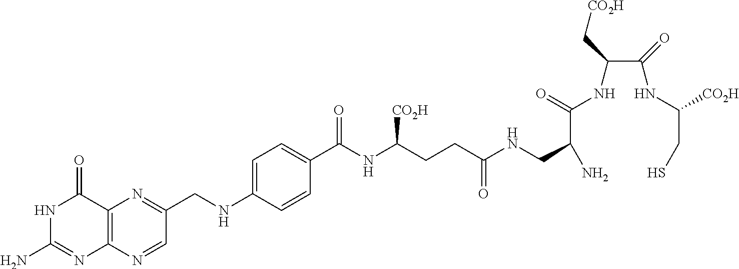 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof, and wherein a radionuclide is bound to the conjugate.
51. The method of any one of clauses 46 to 50, wherein the conjugate is of the formula

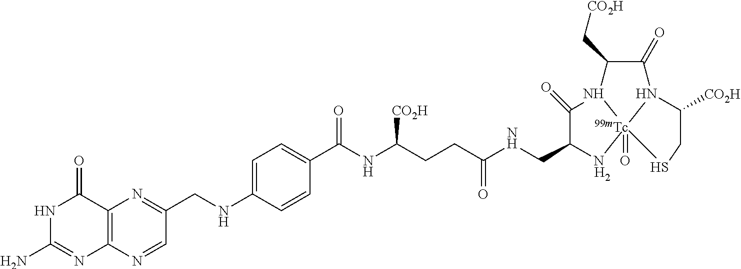

or a pharmaceutically acceptable salt thereof.
52. The method of any one of clauses 39 to 45, wherein the step of determining comprises administering to the patient a conjugate of the formula II

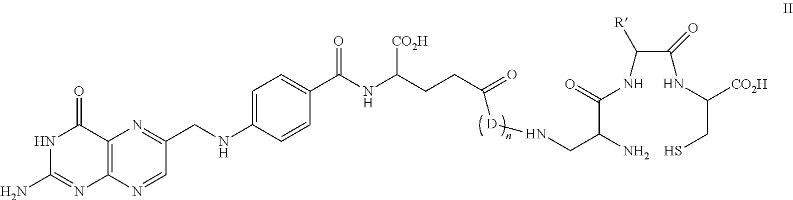

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein a radionuclide is bound to the conjugate.
53. The method of any one of clauses 39 to 45, wherein the step of determining comprises administering a conjugate of the formula III

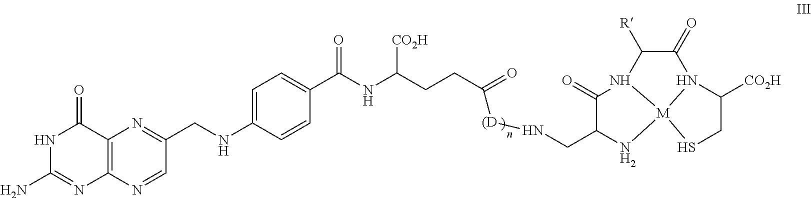

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein M is a cation of a radionuclide.
54. The method of clause 53, wherein M in the conjugate, or a pharmaceutically acceptable salt thereof, is selected from the group consisting of an isotope of gallium, an isotope of indium, an isotope of copper, an isotope of technetium, and an isotope of rhenium.
55. The method of clause 53 or 54, wherein M in the conjugate, or a pharmaceutically acceptable salt thereof, is an isotope of technetium.
56. The method of any one of clauses 52 to 55, wherein the conjugate is of the formula

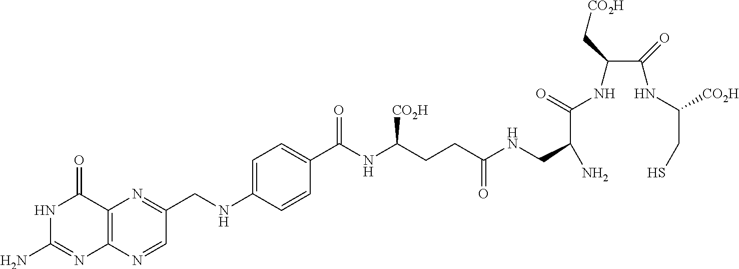 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof, and wherein a radionuclide is bound to the conjugate.
57. The method of any one of clauses 53 to 55, wherein the conjugate is of the formula

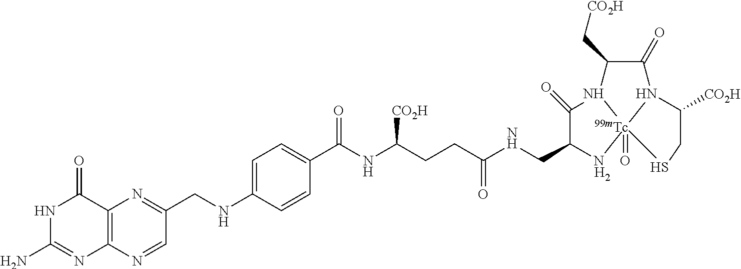

or a pharmaceutically acceptable salt thereof.
58. A method of treating a cancer in a patient in need of such treatment comprising, administering to the patient a therapeutically effective amount of a compound of the formula I

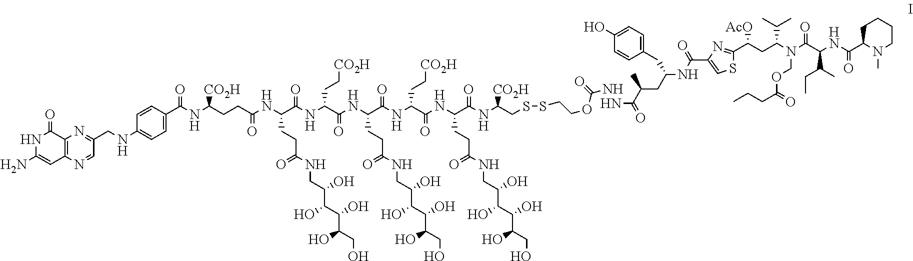 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c2ccc(NCc3cnc4cc(N)[nH]c(=O)c4n3)cc2)C(=O)O)C(=O)O)cs1)C(C)C]

or a pharmaceutically acceptable salt thereof, wherein stable disease results after the compound of the formula I, or a pharmaceutically acceptable salt thereof, is administered.
59. The method of clause 58, wherein the patient has been treated with at least one prior treatment.
60. The method of clause 59, wherein the at least one prior treatment is selected from the group consisting of a chemotherapeutic agent, surgery, radiation therapy, immunotherapy, photodynamic therapy, stem cell therapy, and hyperthermia.
61. The method of clause 59, wherein the at least one prior treatment is a systemic treatment.
62. The method of clause 61, wherein the systemic treatment is selected from the group consisting of palifosfamide, 5-fluorouracil, capecitabine, pemetrexed, cisplatin, carboplatin, gemcitabine, paclitaxel, vinorelbine, eribulin, docetaxel, cyclophosphamide, doxorubicin, regorafinib, and combinations thereof.
63. The method of any one of clauses 58 to 62, wherein the cancer is a folate receptor expressing cancer.
64. The method of any one of clauses 58 to 63, wherein the compound is at least about 98 percent pure.
65. The method of any one of clauses 58 to 64, wherein the cancer is selected from the group consisting of a carcinoma, a sarcoma, a lymphoma, a melanoma, a mesothelioma, a nasopharyngeal carcinoma, a leukemia, an adenocarcinoma, and a myeloma.
66. The method of any one of clauses 58 to 65, wherein the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, leiomyosarcoma, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, cholangiocarcinoma, cancer of the head and neck, malignant neoplasm of parotid gland, appendiceal cancer, duodenal cancer, laryngeal, metastatic hepatocellular carcinoma, metastatic squamous cell left gingiva, Hurthle cell thyroid cancer and adenocarcinoma of the gastroesophageal junction.
67. The method of any one of clauses 58 to 66, wherein the cancer is selected from the group consisting of triple-negative breast cancer, pleural mesothelioma, non-small cell lung cancer, small cell lung cancer, adenocarcinoma of the gastroesophageal junction, ovarian cancer, cancer of the head and neck, malignant neoplasm of parotid gland, appendiceal cancer, duodenal cancer, laryngeal, metastatic hepatocellular carcinoma, metastatic squamous cell left gingiva, leiomyosarcoma and endometrial cancer.
68. The method of any one of clauses 58 to 67, wherein the cancer is triple-negative breast cancer.
69. The method of any one of clauses 58 to 67, wherein the cancer is non-small cell lung cancer.
70. The method of any one of clauses 58 to 67, wherein the cancer is adenocarcinoma of the gastroesophageal junction.
71. The method of any one of clauses 58 to 67, wherein the cancer is ovarian cancer.
72. The method of any one of clauses 58 to 67, wherein the cancer is pleural mesothelioma.
73. The method of any one of clauses 58 to 72, wherein the compound of formula I, or a pharmaceutically acceptable salt thereof, is administered in a parenteral dosage form.
74. The method of clause 73, wherein the parenteral dosage form is selected from the group consisting of intradermal, subcutaneous, intramuscular, intraperitoneal, intravenous, and intrathecal.
75. The method of any one of clauses 58 to 74, wherein the therapeutically effective amount is from about 0.5 mg/m² to about 100.0 mg/m², or about 0.5 mg/m² to about 90.0 mg/m², or about 0.5 mg/m² to about 80.0 mg/m², or about 0.5 mg/m² to about 70.0 mg/m², or about 0.5 mg/m² to about 60.0 mg/m², or about 0.5 mg/m² to about 50.0 mg/m², or 0.5 mg/m² to about 40.0 mg/m², or about 0.5 mg/m² to about 35.0 mg/m², or about 0.5 mg/m² to about 30.0 mg/m², or about 0.5 mg/m² to about 25.0 mg/m², or 0.5 mg/m² to about 24.5 mg/m², or about 0.5 mg/m² to about 24.0 mg/m², or about 0.5 mg/m² to about 23.5 mg/m², or about 0.5 mg/m² to about 23.0 mg/m², or about 0.5 mg/m² to about 22.5 mg/m², or about 0.5 mg/m² to about 22.0 mg/m², or about 0.5 mg/m² to about 21.5 mg/m², or about 0.5 mg/m² to about 21.0 mg/m², or about 0.5 mg/m² to about 20.0 mg/m².
76. The method of any one of clauses 58 to 75, wherein the therapeutically effective amount is from about 0.5 mg/m² to about 19.0 mg/m².
77. The method of any one of clauses 58 to 76, wherein the therapeutically effective amount is from about 0.5 mg/m² to about 18.0 mg/m².
78. The method of any one of clauses 58 to 77, wherein the therapeutically effective amount is from about 0.5 mg/m² to about 17.0 mg/m².
79. The method of any one of clauses 58 to 78, wherein the therapeutically effective amount is from about 0.5 mg/m² to about 16.0 mg/m².
80. The method of any one of clauses 58 to 79, wherein the therapeutically effective amount is from about 0.5 mg/m² to about 15.0 mg/m².
81. The method of any one of clauses 58 to 80, wherein the therapeutically effective amount is from about 0.5 mg/m² to about 14.0 mg/m².
82. The method of any one of clauses 58 to 81, wherein the therapeutically effective amount is from about 0.5 mg/m² to about 13.0 mg/m².
83. The method of any one of clauses 58 to 82, wherein the therapeutically effective amount is from about 0.5 mg/m² to about 12.0 mg/m².
84. The method of any one of clauses 58 to 83, wherein the therapeutically effective amount is from about 0.5 mg/m² to about 11.0 mg/m².
85. The method of any one of clauses 58 to 84, wherein the therapeutically effective amount is from about 0.5 mg/m² to about 10.0 mg/m².
86. The method of any one of clauses 58 to 85, wherein the therapeutically effective amount is from about 0.5 mg/m² to about 9.0 mg/m².
87. The method of any one of clauses 58 to 86, wherein the therapeutically effective amount is from about 0.5 mg/m² to about 8.0 mg/m².
88. The method of any one of clauses 58 to 87, wherein the therapeutically effective amount is from about 0.5 mg/m² to about 7.0 mg/m².
89. The method of any one of clauses 58 to 88, wherein the therapeutically effective amount is from about 0.5 mg/m² to about 6.0 mg/m².
90. The method of any one of clauses 58 to 89, wherein the therapeutically effective amount is from about 0.5 mg/m² to about 5.0 mg/m².
91. The method of any one of clauses 58 to 90, wherein the therapeutically effective amount is from about 0.5 mg/m² to about 4.0 mg/m².
92. The method of any one of clauses 58 to 91, wherein the therapeutically effective amount is from about 0.5 mg/m² to about 3.5 mg/m².
93. The method of any one of clauses 58 to 92, wherein the therapeutically effective amount is from about 0.5 mg/m² to about 3.0 mg/m².
94. The method of any one of clauses 58 to 93, wherein the therapeutically effective amount is from about 0.5 mg/m² to about 2.5 mg/m².
95. The method of any one of clauses 58 to 94, wherein the therapeutically effective amount is from about 0.5 mg/m² to about 2.0 mg/m².
96. The method of any one of clauses 58 to 95, further comprising detecting folate receptor overexpression by the cancer.
97. The method of clause 96, wherein the step of detecting occurs before the step of administering.
98. The method of clause 96 or 97, wherein the detecting is performed by imaging and wherein the imaging is selected from the group consisting of SPECT imaging, PET imaging, IHC, and FISH.
99. The method of any one of clauses 95 to 98, wherein the detecting is performed by SPECT imaging.
100. The method of any one of clauses 58 to 99, further comprising determining the folate receptor status of the patient by imaging.
101. The method of clause 100, wherein the imaging is SPECT imaging.
102. The method of clause 100 or 101, wherein the folate receptor status is based on a measurement of the percentage of evaluable lesions in the patient that are folate receptor positive.
103. The method of any one of clauses 100 to 102, wherein the folate receptor status of the patient correlates with a clinical benefit to the patient.
104. The method of clause 103, wherein the clinical benefit is selected from the group consisting of inhibition of tumor growth, stable disease, a partial response, and a complete response.
105. The method of clause 104, wherein the clinical benefit is stable disease.
106. The method of any one of clauses 96 to 99, wherein the step of detecting comprises administering to the patient a conjugate of the formula II

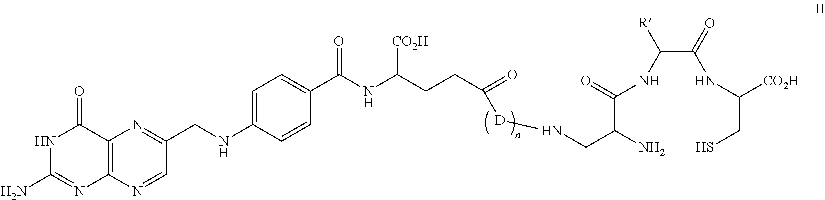

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein a radionuclide is bound to the conjugate.
107. The method of any one of clauses 96 to 99, wherein the step of detecting comprises administering a conjugate of the formula III

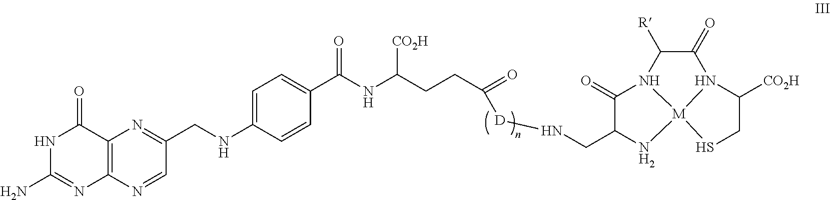

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein M is a cation of a radionuclide.
108. The method of clause 107, wherein M in the conjugate, or a pharmaceutically acceptable salt thereof, is selected from the group consisting of an isotope of gallium, an isotope of indium, an isotope of copper, an isotope of technetium, and an isotope of rhenium.

109. The method of clause 107 or 108, wherein M in the conjugate, or a pharmaceutically acceptable salt thereof, is an isotope of technetium.
110. The method of clause 106, wherein the conjugate is of the formula

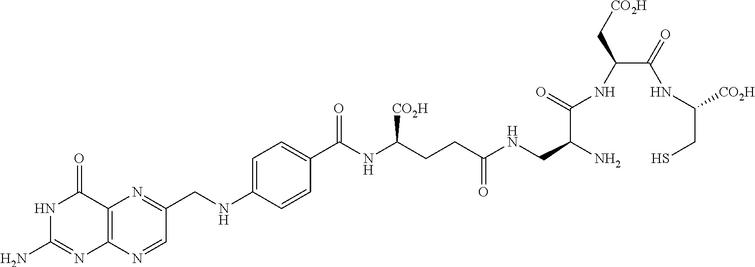 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof, and wherein a radionuclide is bound to the conjugate.
111. The method of any one of clauses 107 to 109, wherein the conjugate is of the formula

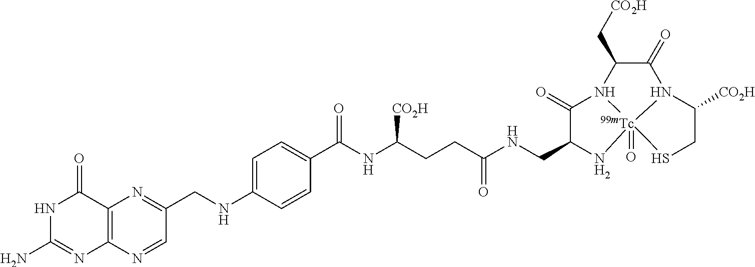

or a pharmaceutically acceptable salt thereof.
112. The method of any one of clauses 100 to 105, wherein the step of determining comprises administering to the patient a conjugate of the formula II

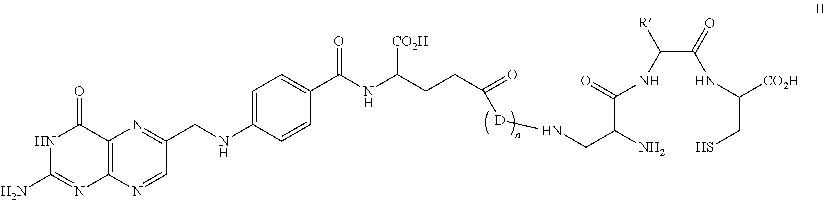

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein a radionuclide is bound to a conjugate.
113. The method of any one of clauses 100 to 105, wherein the step of determining comprises administering a conjugate of the formula III

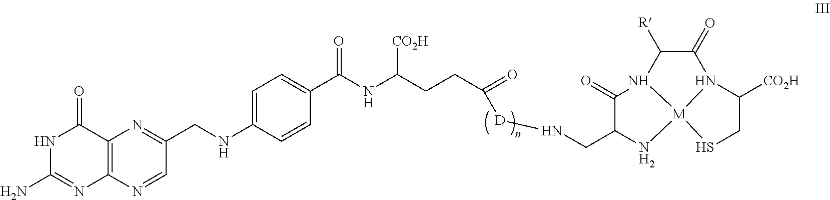

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherin M is a cation of a radionuclide.
114. The method of clause 113, wherein M in the conjugate, or a pharmaceutically acceptable salt thereof, is selected from the group consisting of an isotope of gallium, an isotope of indium, an isotope of copper, an isotope of technetium, and an isotope of rhenium.
115. The method of clause 113 or 114, wherein M in the conjugate, or a pharmaceutically acceptable salt thereof, is an isotope of technetium.
116. The method of any one of clauses 112 to 115, wherein the conjugate is of the formula

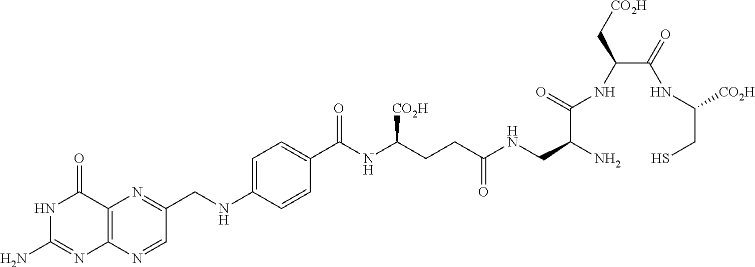 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof, and wherein a radionuclide is bound to the conjugate.
117. The method of any one of clauses 112 to 116, wherein the conjugate is of the formula

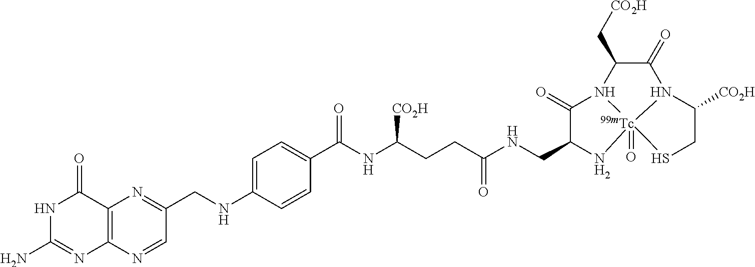

or a pharmaceutically acceptable salt thereof.
118. The method of any one of clauses 60 to 72 or clauses 106 to 117 wherein unlabeled folic acid, or a pharmaceutically acceptable salt thereof, is administered to the patient before the conjugate, or a pharmaceutically acceptable salt thereof, is administered to the patient.
119. A method of treating a folate receptor expressing cancer in a patient in need of such treatment comprising, administering to the patient a therapeutically effective amount of a compound of the formula I

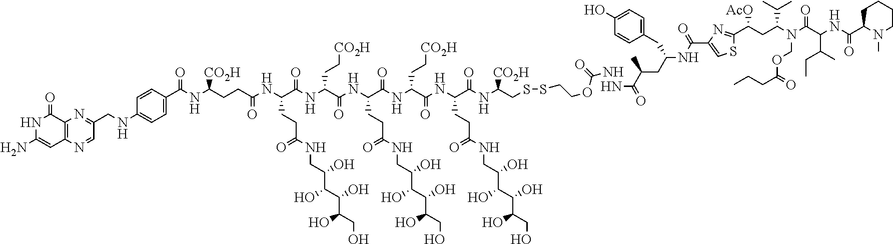 [SMILES: CCCC(=O)OCN(C(=O)C(NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c2ccc(NCc3cnc4cc(N)[nH]c(=O)c4n3)cc2)C(=O)O)C(=O)O)cs1)C(C)C]

or a pharmaceutically acceptable salt thereof, wherein the patient has been identified as having a folate receptor-expressing cancer.

### BRIEF DESCRIPTION OF THE DRAWINGS

FIG. 1: Antitumor effects of paclitaxel, EC145 and EC1456 on paclitaxel resistant KB-PR10 tumors. KB-PR10 tumor cells (1×10⁶) were inoculated subcutaneously into nu/nu mice and therapy started on randomized mice. Each curve shows the average volume of 5 tumors. ▪, Control; ▴, paclitaxel, 20 mg/kg, TIW×2; ♦, EC145, 2 μmol/kg, TIW×2; , EC1456, 1 μmol/kg, TIW×2.

FIG. 2: Antitumor effects of EC1456 in mice bearing LU2505 tumors. LU2505 tumor cells were inoculated subcutaneously into female Balb/c nu/nu mice. Each curve shows the average volume of 5 tumors. ▪, LU2505 Control; , EC1456, 2 μmol/kg, BIW×2; ▴, EC1456, 2 μmol/kg, SIW×2.

FIG. 3: Antitumor effects of EC1456 in mice bearing LU1147 tumors. LU1147 tumor cells were inoculated subcutaneously into female Balb/c nu/nu mice. Each curve shows the average volume of 5 tumors. ▪, LU1147 Control; , EC1456, 2 μmol/kg, BIW×2; A, EC1456, 2 μmol/kg, SIW×2.

FIG. 4: Antitumor effects of EC1456 in mice bearing large KB tumors. EC1456 at 2 μmol/kg (three times a week for two weeks) produced excellent anti-tumor activity with 100% cures in both the 1000 and 1400 mm³ groups. a. ▪, Control; b. , 700 mm³ tumor; c. , 1000 mm³ tumor; d. , 1400 mm³ tumor.

FIG. 5: Antitumor effects of EC1456 in mice bearing ST040 Endometrial tumors. Treatment with 15 mg/kg of Paclitaxel (once a week for two weeks) produced minimal anti-tumor activity with zero animals exhibiting stable disease. EC1456 at 1.5 μmol/kg (two times a week for two weeks) and 3 μmol/kg (once a week for two weeks) produced slightly better anti-tumor activity with 2 animals exhibiting stable disease/1 animal exhibiting PR and 2 animals exhibiting stable disease respectively. a. ▪, Control {0,0,0,0}/5; b. ▾, Paclitaxel 15 mg/kg, SIW×2 {0,0,0,0}/3; c. ▴, EC1456, 1.5 μmoles/kg BIW×2 {2,1,0,0}/5; d. ▴, EC1456, 3 μmoles/kg SIW×2 {2,1,0,0}/5. Dotted line represents last day of dosing. All result reported as stable disease, partial response, complete response, cure {SD, PR, CR, Cure}.

FIG. 6: Antitumor effects of EC1456 in mice bearing ST502 TNBC tumors. Treatment with 1 mg/kg of Eribulin mesylate (once a week for two weeks) produced minimal anti-tumor activity with 1 animal exhibiting stable disease/1 animal exhibiting PR. EC1456 at 2 μmol/kg (two times a week for two weeks) and 4 μmol/kg (once a week for two weeks) produced no anti-tumor activity. a. ▪, Control {0,0,0,0}/7; b. ▾, Eribulin mesylate, 1 mg/kg, SIW×2 {1,1,0,0}/7; c. ▴, EC1456, 2 μmoles/kg BIW×2 {0,0,0,0}/7; d. ▴, EC1456, 4 μmoles/kg SIW×2 {0,0,0,0}/7. Dotted line represents last day of dosing. All result reported as {SD, PR, CR, Cure}.

FIG. 7: Antitumor effects of EC1456 in mice bearing ST738 TNBC tumors. Treatment with 1 mg/kg of Eribulin mesylate (once a week for two weeks) produced some anti-tumor activity with 5 animals exhibiting stable disease/2 PR's. EC1456 at 2 μmol/kg (two times a week for two weeks) and 4 μmol/kg (once a week for two weeks) also produced some anti-tumor activity with 2 animals exhibiting stable disease/3 animals exhibiting PR's and 2 animals exhibiting stable disease/5 animals exhibiting PR's. a. ▪, Control {0,0,0,0}/7; b. ▾, Eribulin mesylate, 1 mg/kg, SIW×2 {5,2,0,0}/7; c. ▴, EC1456, 2 μmoles/kg BIW×2 {2,3,0,2}/7; d. ▴, EC1456, 4 μmoles/kg SIW×2 {2,5,0,0}/7. Dotted line represents last day of dosing. All result reported as {SD, PR, CR, Cure}.

FIG. 8: Antitumor effects of EC1456 in mice bearing ST024 Ovarian tumors. Treatment with 15 mg/kg of Paclitaxel (once a week for two weeks) produced no anti-tumor activity. EC1456 at 2 μmol/kg (two times a week for two weeks) and 4 μmol/kg (once a week for two weeks) produced curative (100% animals exhibiting cures) anti-tumor activity. a. ▪, Control {0,0,0,0}/7; b. ▾, Paclitaxel 15 mg/kg, SIW×2 {0,0,0,0}/7; c. ▴, EC1456, 2 μmoles/kg BIW×2 {0,1,0,6}/5; d. ▴, EC1456, 4 μmoles/kg SIW×2 {0,2,0,5}/5. Dotted line represents last day of dosing. All result reported as {SD, PR, CR, Cure}.

### DEFINITIONS

In accordance with the invention, “functionally active folate receptors” means folate receptors that bind folate.

In accordance with the invention, “clinical benefit” means a response of a patient to treatment with Compound I where the response includes overall survival of the patient, ability to receive four or more cycles of therapy (e.g., four weeks of therapy) with Compound I, inhibition of tumor growth, stable disease, a partial response, and/or a complete response, among other clinical benefits defined by the Food and Drug Administration in the United States of America.

In accordance with the invention, “inhibition of tumor growth” means reduction in tumor size, complete disappearance of a tumor, or growth of a patient tumor of less than 30% over the course of therapy with Compound I.

In accordance with the invention, “stable disease” means no material progression of disease in a patient over the course of therapy with Compound I.

In accordance with the invention, “a partial response” means a decrease in tumor size of 30% or greater in a patient treated with Compound I.

In accordance with the invention, “a complete response” means the disappearance of detectable disease in a patient treated with Compound I.

In accordance with the invention, “prior treatment” means the patient has been treated with at least one prior treatment known in the art. It will be appreciated that a prior treatment can be any treatment known to those of skill in the art, including, but not limited, chemotherapeutic agent, surgery, radiation therapy, immunotherapy, photodynamic therapy, stem cell therapy, hyperthermia, and the like. Prior treatments can include systemic treatments including, but not limited to treatment with palifosfamide, 5-fluorouracil, capecitabine, pemetrexed, cisplatin, carboplatin, gemcitabine, paclitaxel, vinorelbine, eribulin, docetaxel, cyclophosphamide, doxorubicin, regorafinib, and combinations thereof.

In accordance with the inventions, the term “alkyl” includes a chain of carbon atoms, which is optionally branched. As used herein, the term “alkenyl” and “alkynyl” includes a chain of carbon atoms, which is optionally branched, and includes at least one double bond or triple bond, respectively. It is to be understood that alkynyl may also include one or more double bonds. It is to be further understood that in certain embodiments, alkyl is advantageously of limited length, including C₁-C₂₄, C₁-C₁₂, C₁-C₈, C₁-C₆, and C₁-C₄. Illustratively, such particularly limited length alkyl groups, including C₁-C₈, C₁-C₆, and C₁-C₄ may be referred to as lower alkyl. It is to be further understood that in certain embodiments alkenyl and/or alkynyl may each be advantageously of limited length, including C₂-C₂₄, C₂-C₁₂, C₂-C₈, C₂-C₆, and C₂-C₄. Illustratively, such particularly limited length alkenyl and/or alkynyl groups, including C₂-C₈, C₂-C₆, and C₂-C₄ may be referred to as lower alkenyl and/or alkynyl. It is appreciated herein that shorter alkyl, alkenyl, and/or alkynyl groups may add less lipophilicity to the compound and accordingly will have different pharmacokinetic behavior. In embodiments of the invention described herein, it is to be understood, in each case, that the recitation of alkyl refers to alkyl as defined herein, and optionally lower alkyl. In embodiments of the invention described herein, it is to be understood, in each case, that the recitation of alkenyl refers to alkenyl as defined herein, and optionally lower alkenyl. In embodiments of the invention described herein, it is to be understood, in each case, that the recitation of alkynyl refers to alkynyl as defined herein, and optionally lower alkynyl. Illustrative alkyl, alkenyl, and alkynyl groups are, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like, and the corresponding groups containing one or more double and/or triple bonds, or a combination thereof.

In accordance with the invention, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium. As used herein, the term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

In accordance with the invention, the term “aryl” includes monocyclic and polycyclic aromatic carbocyclic groups having from 6 to 14 ring carbon atoms, each of which may be optionally substituted. Illustrative aromatic carbocyclic groups described herein include, but are not limited to, phenyl, naphthyl, and the like. In accordance with the invention, the term “heteroaryl” includes aromatic heterocyclic groups, having from 5 to 10 ring atoms, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like. In accordance with the invention, the term “heteroarylalkyl” includes a combination of an “alkyl” group as described herein with a “heteroaryl” group described herein. In accordance with the invention, the term “arylalkyl” includes a combination of an “alkyl” group as described herein with a “aryl” group described herein, for example a benzyl group.

In accordance with the invention, the term “amino” includes the group NH₂, alkylamino, aminoalkyl and dialkylamino, where the two alkyl groups in dialkylamino may be the same or different, i.e. alkylalkylamino. Illustratively, amino includes methylamino, ethylamino, dimethylamino, methylethylamino, and the like. In addition, it is to be understood that when amino modifies or is modified by another term, such as aminoalkyl, or acylamino, the above variations of the term amino are included therein. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

In accordance with the invention, the term “hydroxy” means —OH, and hydrozy can be appended to numerous groups contemplated herein to form, for example, hydroxylalkyl, alkyloxy, alkenyloxy, alkynyloxy, heteroalkyloxy, heteroalkenyloxy, heteroalkynyloxy, cycloalkyloxy, cycloalkenyloxy, cycloheteroalkyloxy, cycloheteroalkenyloxy, aryloxy, arylalkyloxy, arylalkenyloxy, arylalkynyloxy, heteroaryloxy, heteroarylalkyloxy, heteroarylalkenyloxy, heteroarylalkynyloxy, acyloxy, and the like, each of which is optionally substituted.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative substituents include, but are not limited to, a radical —(CH₂)ₓZ^{X}, where x is an integer from 0-6 and Z^{X} is selected from the group consisting of halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, and nitro; or Z^{X} is selected from the group consisting of—-CO₂R⁴ and —CONR⁵R⁶, where R⁴, R⁵, and R⁶ are each independently selected in each occurrence from hydrogen, C₁-C₆ alkyl, aryl-C₁-C₆ alkyl, and heteroaryl—C₁-C₆ alkyl.

In accordance with the invention, the term “administering” as used herein includes all means of introducing the compounds and folate-imaging agent conjugates described herein to the patient, including, but not limited to, oral (po), intravenous (iv), intramuscular (im), subcutaneous (sc), transdermal, inhalation, buccal, ocular, sublingual, vaginal, rectal, and the like. The compounds and folate-imaging agent conjugates described herein may be administered in unit dosage forms and/or formulations containing conventional nontoxic pharmaceutically-acceptable carriers, adjuvants, and vehicles.

### Detailed Description of the Illustrative Embodiments

In accordance with Applicants' invention described herein, the embodiments of the numbered clauses provided in the summary above, or any combination thereof, are contemplated for combination with any of the embodiments described in the Detailed Description section of this patent application.

In one embodiment, the methods described herein can be used for both human clinical medicine and veterinary applications. Thus, a “patient” can be administered the compounds or folate-imaging agent conjugates described herein, and can be human or, in the case of veterinary applications, can be a laboratory, agricultural, domestic, or wild animal. In one aspect, the patient can be a human, a laboratory animal such as a rodent (e.g., mice, rats, hamsters, etc.), a rabbit, a monkey, a chimpanzee, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

In various embodiments, the cancers described herein can be a cancer cell population that is tumorigenic, including benign tumors and malignant tumors, or the cancer can be non-tumorigenic. The cancer can arise spontaneously or by such processes as mutations present in the germline of the patient or somatic mutations, or the cancer can be chemically-, virally-, or radiation-induced. Cancers applicable to the invention described herein include, but are not limited to, a carcinoma, a sarcoma, a lymphoma, a melanoma, a mesothelioma, a nasopharyngeal carcinoma, a leukemia, an adenocarcinoma, and a myeloma.

In some aspects the cancers can be lung cancer, bone cancer, pancreatic cancer, skin cancer, cancer of the head, cancer of the neck, cutaneous melanoma, intraocular melanoma uterine cancer, ovarian cancer, endometrial cancer, leiomyosarcoma, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, cholangiocarcinoma, cancer of the head and neck, malignant neoplasm of parotid gland, appendiceal cancer, duodenal cancer, laryngeal, metastatic hepatocellular carcinoma, metastatic squamous cell left gingiva, Hurthle cell thyroid cancer or adenocarcinoma of the gastroesophageal junction.

Compound I has the formula

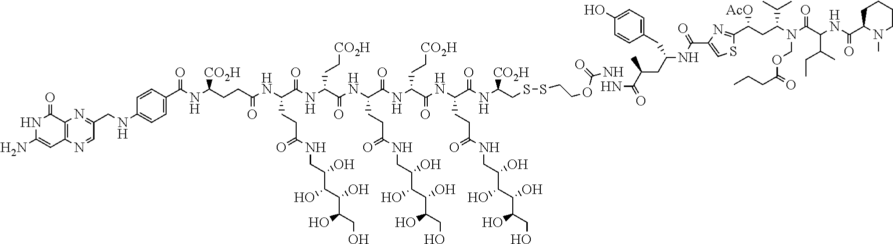 [SMILES: CCCC(=O)OCN(C(=O)C(NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c2ccc(NCc3cnc4cc(N)[nH]c(=O)c4n3)cc2)C(=O)O)C(=O)O)cs1)C(C)C]

and the conjugates described herein include the following formulas

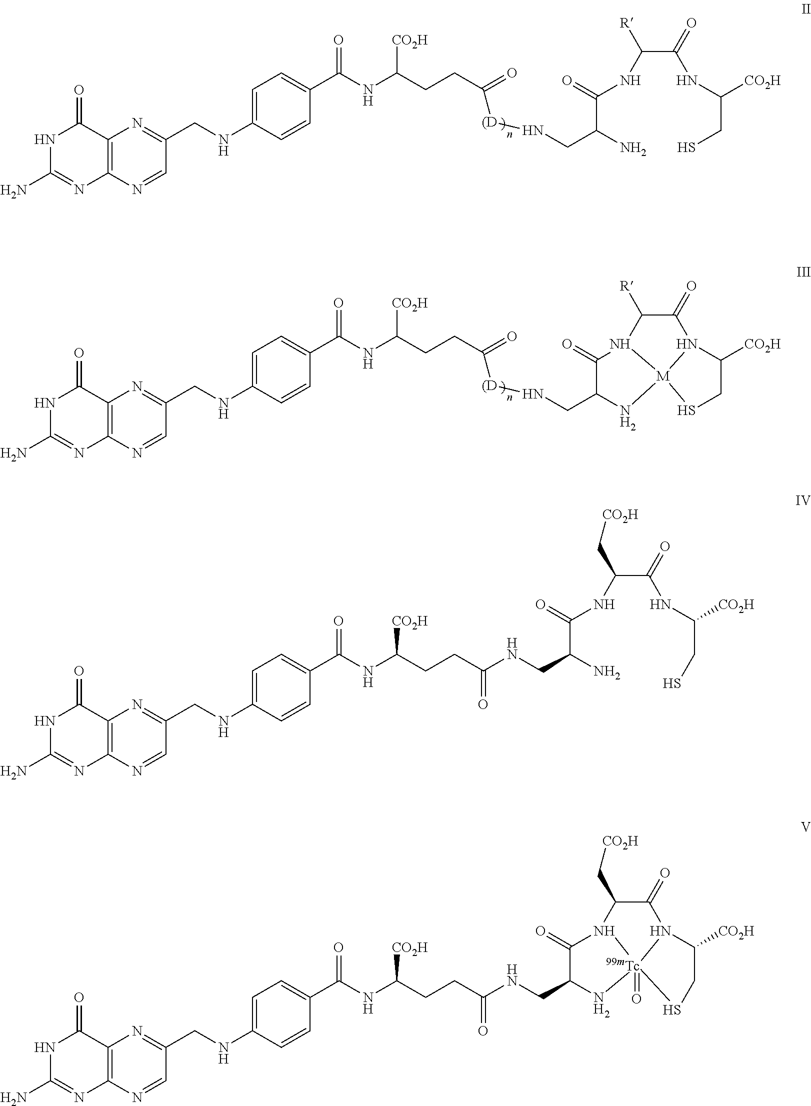

In other embodiments, any of a variety of folate-imaging agent conjugates detectable by PET imaging, SPECT imaging, and the like can be used. The exact manner of imaging is not limited to the imaging agents described herein. Collectively, the folate-imaging agent conjugates useful for imaging described herein, including those described by formulas and the agents useful for PET imaging, SPECT imaging, etc. are referred to as “folate-imaging agent conjugates.”

In one embodiment, the compounds and folate-imaging agent conjugates described herein bind to over-expressed folate receptors on cancer cells. In one illustrative aspect, the compounds and folate-imaging agent conjugates are capable of differentially binding to folate receptors on cancer cells compared to normal cells due to preferential expression (or over-expression) of the folate receptor on the cancer cells.

In one illustrative aspect, the chemical linkage (e.g. “D” or “divalent linker”) in the conjugate described herein can be a direct linkage or the linkage can be through an intermediary linker. In one embodiment, if present, an intermediary linker can be any biocompatible linker known in the art. In one illustrative embodiment, the divalent linker comprises about 1 to about 30 carbon atoms. In another illustrative embodiment, the divalent linker comprises about 2 to about 20 carbon atoms. In other embodiments, lower molecular weight divalent linkers (i.e., those having an approximate molecular weight of about 30 to about 300) are employed.

In one embodiment, the divalent linker comprises a heteroatom directly bonded to the folate or to the imaging agent. In one embodiment, the heteroatom is nitrogen. In another embodiment, the divalent linker comprises an optionally-substituted diaminoalkylene. In one embodiment, the optionally-substituted diaminoalkylene is a diaminoacid. In another embodiment, the divalent linker comprises one or more optionally-substituted diaminoalkylene moieties, and one or more optionally-substituted amino acids. In one illustrative example, the divalent linker comprises glutamic acid.

In another illustrative embodiment, the divalent linker includes one or more amino acids. In one variation, the divalent linker includes a single amino acid. In another variation, the divalent linker includes a peptide having from 2 to about 50, 2 to about 30, or 2 to about 20 amino acids. In another variation, the divalent linker includes a peptide having from about 4 to about 8 amino acids. Such amino acids are illustratively selected from the naturally occurring amino acids, or stereoisomers thereof. In another embodiment, the amino acid may also be any other amino acid, such as any amino acid having the general formula:

—N(R¹)—(CR²R³)_{q-}C(O)—

where R¹ is hydrogen, alkyl, acyl, or a suitable nitrogen protecting group, R² and R³ in the amino acid are hydrogen or a substituent, each of which is independently selected in each occurrence, and q is an integer such as 1, 2, 3, 4, or 5. Illustratively, R² and/or R³ in the amino acid independently correspond to, but are not limited to, hydrogen or the side chains present on naturally occurring amino acids, such as methyl, benzyl, hydroxymethyl, thiomethyl, carboxyl, carboxylmethyl, guanidinopropyl, and the like, and derivatives and protected derivatives thereof. The above described formula includes all stereoisomeric variations. For example, the amino acid may be selected from asparagine, aspartic acid, cysteine, glutamic acid, lysine, glutamine, arginine, serine, ornithine, threonine, and the like. In one variation, the divalent linker includes at least 2 amino acids selected from of asparagine, aspartic acid, cysteine, glutamic acid, lysine, glutamine, arginine, serine, ornithine, and threonine. In another variation, the divalent linker includes between 2 and about 5 amino acids selected from asparagine, aspartic acid, cysteine, glutamic acid, lysine, glutamine, arginine, serine, ornithine, and threonine. In another variation, the divalent linker includes a tripeptide, tetrapeptide, pentapeptide, or hexapeptide consisting of amino acids selected from aspartic acid, cysteine, glutamic acid, lysine, arginine, and ornithine, and combinations thereof.

In another embodiment, the divalent linker may also include one or more spacer linkers. Illustrative spacer linkers are shown in the following table. The following non-limiting, illustrative spacer linkers are described where * indicates the point of attachment to the folate or the imaging moiety portion of the imaging agent in the conjugate.

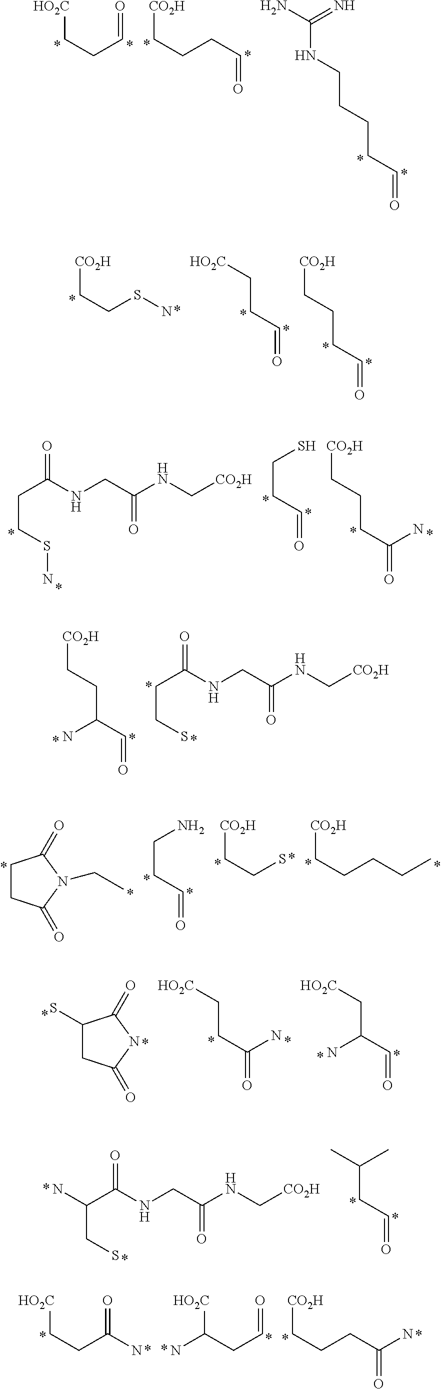

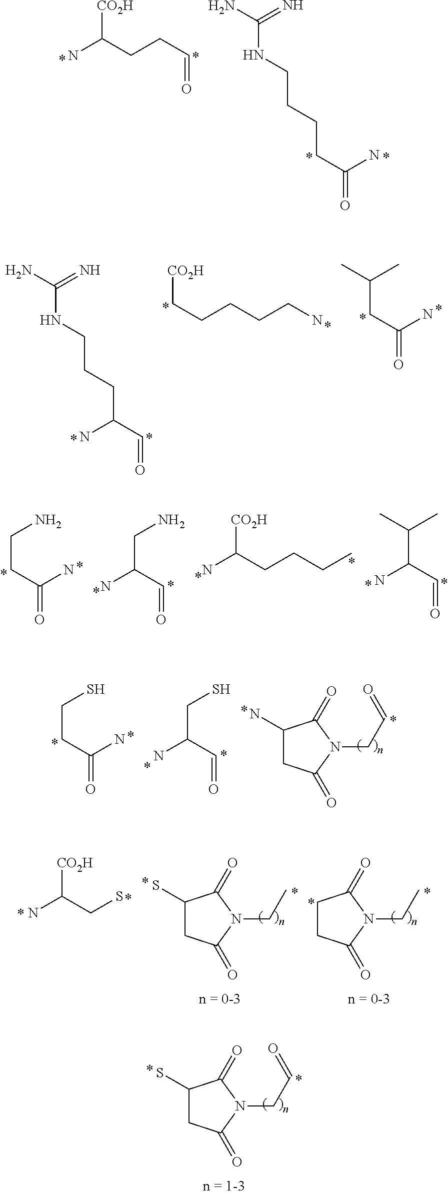

In other embodiments of the methods described herein, pharmaceutically acceptable salts of the compounds and folate-imaging agent conjugates described herein are provided. Pharmaceutically acceptable salts of the compounds and folate-imaging agent conjugates described herein include acid addition and base salts thereof.

Suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts of the compounds and folate-imaging agent conjugates described herein are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts.

In one embodiment, the compounds and folate-imaging agent conjugates described herein may be administered as a formulation in association with one or more pharmaceutically acceptable carriers. The carriers can be excipients. The choice of carrier will to a large extent depend on factors such as the particular mode of administration, the effect of the carrier on solubility and stability, and the nature of the dosage form. Pharmaceutical compositions suitable for the delivery of compounds and folate-imaging agent conjugates described herein and methods for their preparation will be readily apparent to those skilled in the art. Such compositions and methods for their preparation may be found, for example, in Remington: The Science & Practice of Pharmacy, 21th Edition (Lippincott Williams & Wilkins, 2005), incorporated herein by reference.

In one illustrative aspect, a pharmaceutically acceptable carrier includes any and all solvents, dispersion media, coatings, antibacterial and antifungal agents, isotonic and absorption delaying agents, and the like, and combinations thereof, that are physiologically compatible. In some embodiments, the carrier is suitable for parenteral administration. Pharmaceutically acceptable carriers include sterile aqueous solutions or dispersions and sterile powders for the extemporaneous preparation of sterile injectable solutions or dispersions. Supplementary active compounds can also be incorporated into compositions of the invention.

In various embodiments, liquid formulations may include suspensions and solutions. Such formulations may comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid.

In one embodiment, an aqueous suspension may contain the active materials in admixture with appropriate excipients. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions may also contain one or more preservatives, for example, ascorbic acid, ethyl, n-propyl, or p-hydroxybenzoate; or one or more coloring agents.

In one illustrative embodiment, dispersible powders and granules suitable for preparation of an aqueous suspension by the addition of water provide the active ingredient in admixture with a dispersing or wetting agent, suspending agent and one or more preservatives. Additional excipients, for example, coloring agents, may also be present.

Suitable emulsifying agents may be naturally-occurring gums, for example, gum acacia or gum tragacanth; naturally-occurring phosphatides, for example, soybean lecithin; and esters including partial esters derived from fatty acids and hexitol anhydrides, for example, sorbitan mono-oleate, and condensation products of the said partial esters with ethylene oxide, for example, polyoxyethylene sorbitan monooleate.

In other embodiments, isotonic agents, for example, sugars, polyalcohols such as mannitol, sorbitol, or sodium chloride can be included in the composition. Prolonged absorption of injectable compositions can be brought about by including in the composition an agent which delays absorption, for example, monostearate salts and gelatin.

Illustrative formats for oral administration include tablets, capsules, elixirs, syrups, and the like.

Depending upon the cancer type as described herein, the route of administration and/or whether the compounds and/or folate-imaging agent conjugates are administered locally or systemically, a wide range of permissible dosages are contemplated herein, including doses falling in the range from about 1 μg/kg to about 1 g/kg. The dosages may be single or divided, and may administered according to a wide variety of protocols, including q.d., b.i.d., t.i.d., or even every other day, biweekly (b.i.w.), once a week, once a month, once a quarter, and the like.

In each of these cases it is understood that the therapeutically effective amounts described herein correspond to the instance of administration, or alternatively to the total daily, weekly, month, or quarterly dose, as determined by the dosing protocol.

In one aspect, a compound or a folate-imaging agent conjugate as described herein may be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. Suitable means for parenteral administration include needle (including microneedle) injectors, needle-free injectors and infusion techniques.

In one illustrative aspect, parenteral formulations are typically aqueous solutions which may contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water. In other embodiments, any of the liquid formulations described herein may be adapted for parenteral administration of the compounds or folate-imaging agent conjugates described herein. The preparation of parenteral formulations under sterile conditions, for example, by lyophilization under sterile conditions, may readily be accomplished using standard pharmaceutical techniques well known to those skilled in the art. In one embodiment, the solubility of a compound or a folate-imaging agent conjugate used in the preparation of a parenteral formulation may be increased by the use of appropriate formulation techniques, such as the incorporation of solubility-enhancing agents.

In various embodiments, formulations for parenteral administration may be formulated for immediate and/or modified release. In one illustrative aspect, active agents of the invention (i.e., the compounds or folate-imaging agent conjugates) may be administered in a time release formulation, for example in a composition which includes a slow release polymer. The active compounds or folate-imaging agent conjugates can be prepared with carriers that will protect the compound or folate-imaging agent conjugate against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art. In another embodiment, the compounds or folate-imaging agent conjugates described herein or compositions comprising the compounds or folate-imaging agent conjugates may be continuously administered, where appropriate.

In one embodiment, a kit is provided. If a combination of active compounds and folate-imaging agent conjugates is to be administered, two or more pharmaceutical compositions may be combined in the form of a kit suitable for sequential administration or co-administration of the compositions. Such a kit comprises two or more separate pharmaceutical compositions, at least one of which contains a compound or folate-imaging agent conjugate described herein, and means for separately retaining the compositions, such as a container, divided bottle, or divided foil packet. In another embodiment, compositions comprising one or more compounds or folate-imaging agent conjugates described herein, in containers having labels that provide instructions for use of the compounds or folate-imaging agent conjugates for patient selection and/or treatment are provided.

In one embodiment, sterile injectable solutions can be prepared by incorporating the active agent in the required amount in an appropriate solvent with one or a combination of ingredients described above, as required, followed by filtered sterilization. Typically, dispersions are prepared by incorporating the active compound or folate-imaging agent conjugate into a sterile vehicle which contains a dispersion medium and any additional ingredients of those described above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof, or the ingredients may be sterile-filtered together.

The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. In one embodiment, the proper fluidity can be maintained, for example, by the use of a coating such as lecithin, by the maintenance of the required particle size in the case of dispersion and by the use of surfactants.

Any effective regimen for administering Compound I can be used. For example, Compound I can be administered as single doses, or the doses can be divided and administered as a multiple-dose daily regimen. Further, a staggered regimen, for example, one to five days per week can be used as an alternative to daily treatment, and for the purpose of the methods described herein, such intermittent or staggered daily regimen is considered to be equivalent to every day treatment and is contemplated. In one illustrative embodiment the patient is treated with multiple injections of Compound I to treat the cancer. In one embodiment, the patient is injected multiple times (preferably about 2 up to about 50 times) with Compound I, for example, at 12-72 hour intervals or at 48-72 hour intervals. Additional injections of Compound I can be administered to the patient at an interval of days or months after the initial injections(s) and the additional injections can prevent recurrence of the cancer.

Any suitable course of therapy with Compound I can be used. In one embodiment, individual doses and dosage regimens are selected to provide a total dose administered during a month of about 15 mg. In one illustrative example, Compound I is administered in a single daily dose administered five days a week, in weeks 1, 2, and 3 of each 4 week cycle, with no dose administered in week 4. In an alternative example, Compound I is administered in a single daily dose administered three days a week, of weeks 1, and 3 of each 4 week cycle, with no dose administered in weeks 2 and 4. In an alternative example, Compound I is administered biweekly on weeks 1 and 2, i.e. on days 1, 4, 8, 11, of a 3-week cycle. In an alternative example, Compound I is administered and once weekly on weeks 1 and 2, i.e. days 1 and 8 of a 3-week cycle. The unitary daily dosage of Compound I can vary significantly depending on the patient condition, the cancer being treated, the route of administration of Compound I and tissue distribution, and the possibility of co-usage of other therapeutic treatments, such as radiation therapy or additional drugs in combination therapies. The effective amount to be administered to a patient is based on body surface area, mass, and physician assessment of patient condition.

Therapeutically effective doses (also referred to herein as “therapeutically effective amount”) can range, for example, from about 0.5 mg/m² to about 100.0 mg/m². The therapeutically effective doses described herein also include ranges of about 0.5 mg/m² to about 90.0 mg/m², about 0.5 mg/m² to about 80.0 mg/m², about 0.5 mg/m² to about 70.0 mg/m², about 0.5 mg/m² to about 60.0 mg/m², about 0.5 mg/m² to about 50.0 mg/m², 0.5 mg/m² to about 40.0 mg/m², about 0.5 mg/m² to about 35.0 mg/m², about 0.5 mg/m² to about 30.0 mg/m², about 0.5 mg/m² to about 25.0 mg/m², 0.5 mg/m² to about 24.5 mg/m², about 0.5 mg/m² to about 24.0 mg/m², about 0.5 mg/m² to about 23.5 mg/m², about 0.5 mg/m² to about 23.0 mg/m², about 0.5 mg/m² to about 22.5 mg/m², about 0.5 mg/m² to about 22.0 mg/m², about 0.5 mg/m² to about 21.5 mg/m², about 0.5 mg/m² to about 21.0 mg/m², about 0.5 mg/m² to about 20.0 mg/m², about 0.5 mg/m² to about 19.5 mg/m², about 0.5 mg/m² to about 19.0 mg/m², about 0.5 mg/m² to about 18.5 mg/m², about 0.5 mg/m² to about 18.0 mg/m², about 0.5 mg/m² to about 17.5 mg/m², about 0.5 mg/m² to about 17.0 mg/m², about 0.5 mg/m² to about 16.5 mg/m², about 0.5 mg/m² to about 16.0 mg/m², about 0.5 mg/m² to about 15.5 mg/m², about 0.5 mg/m² to about 15.0 mg/m², about 0.5 mg/m² to about 14.5 mg/m², about 0.5 mg/m² to about 14.0 mg/m², about 0.5 mg/m² to about 13.5 mg/m², about 0.5 mg/m² to about 13.0 mg/m², about 0.5 mg/m² to about 12.5 mg/m², about 0.5 mg/m² to about 12.0 mg/m², about 0.5 mg/m² to about 11.5 mg/m², about 0.5 mg/m² to about 11.0 mg/m², about 0.5 mg/m² to about 10.5 mg/m², about 0.5 mg/m² to about 10.0 mg/m², about 0.5 mg/m² to about 9.5 mg/m², about 0.5 mg/m² to about 9.0 mg/m², about 0.5 mg/m² to about 8.5 mg/m², about 0.5 mg/m² to about 8.0 mg/m², about 0.5 mg/m² to about 7.5 mg/m² about 0.5 mg/m² to about 7.0 mg/m², about 0.5 mg/m² to about 6.5 mg/m², about 0.5 mg/m² to about 6.0 mg/m², about 0.5 mg/m² to about 5.5 mg/m², about 0.5 mg/m² to about 5.0 mg/m², about 0.5 mg/m² to about 4.5 mg/m², about 0.5 mg/m² to about 4.0 mg/m², about 0.5 mg/m² to about 3.5 mg/m², about 0.5 mg/m² to about 3.0 mg/m², about 0.5 mg/m² to about 2.5 mg/m², about 0.5 mg/m² to about 2.0 mg/m², about 0.5 mg/m² to about 1.5 mg/m², about 1.0 mg/m² to about 19.5 mg/m², about 1.0 mg/m² to about 19.0 mg/m², about 1.0 mg/m² to about 18.5 mg/m², about 1.0 mg/m² to about 18.0 mg/m², about 1.0 mg/m² to about 17.5 mg/m², about 1.0 mg/m² to about 17.0 mg/m², about 1.0 mg/m² to about 16.5 mg/m², about 1.0 mg/m² to about 16.0 mg/m², about 1.0 mg/m² to about 15.5 mg/m², about 1.0 mg/m² to about 15.0 mg/m², about 1.0 mg/m² to about 14.5 mg/m², about 1.0 mg/m² to about 14.0 mg/m², about 1.0 mg/m² to about 13.5 mg/m², about 1.0 mg/m² to about 13.0 mg/m², about 1.0 mg/m² to about 12.5 mg/m², about 1.0 mg/m² to about 12.0 mg/m², about 1.0 mg/m² to about 11.5 mg/m², about 1.0 mg/m² to about 11.0 mg/m², about 1.0 mg/m² to about 10.5 mg/m², about 1.0 mg/m² to about 10.0 mg/m², about 1.0 mg/m² to about 9.5 mg/m², about 1.0 mg/m² to about 9.0 mg/m², about 1.0 mg/m² to about 8.5 mg/m², about 1.0 mg/m² to about 8.0 mg/m², about 1.0 mg/m² to about 7.5 mg/m², about 1.0 mg/m² to about 7.0 mg/m², about 1.0 mg/m² to about 6.5 mg/m², about 1.0 mg/m² to about 6.0 mg/m², about 1.0 mg/m² to about 5.5 mg/m², about 1.0 mg/m² to about 5.0 mg/m², about 1.0 mg/m² to about 4.5 mg/m², about 1.0 mg/m² to about 4.0 mg/m², about 1.0 mg/m² to about 3.5 mg/m², about 1.0 mg/m² to about 3.0 mg/m², about 1.0 mg/m² to about 2.5 mg/m², about 1.0 mg/m² to about 2.0 mg/m², and about 1.0 mg/m² to about 1.5 mg/m². One of skill in the art will readily appreciate that the therapeutically effective dose may vary within the various ranges provided above based on the factors noted above. The therapeutically effective dose for any particular patient or group of patients may be any number value between about 0.5 mg/m² and about 100.0 mg/m², including but not limited to 1.0 mg/m², 1.5, mg/m², 2.0 mg/m², 2.5 mg/m², 3.0 mg/m², 3.5 mg/m², 4.0 mg/m², 4.5 mg/m², 5.0 mg/m², 5.5 mg/m², 6.0 mg/m², 6.5 mg/m², 7.0 mg/m², 7.5 mg/m², 8.0 mg/m², 8.5 mg/m², 9.0 mg/m², 9.5 mg/m², 10.0 mg/m², 10.5 mg/m², 11.0 mg/m², 11.5 mg/m², 12.0 mg/m², 12.5 mg/m², 13.0 mg/m², 13.5 mg/m², 14.0 mg/m², 14.5 mg/m², 15.0 mg/m², 15.5 mg/m², 16.0 mg/m², 16.5 mg/m², 17.0 mg/m², 17.5 mg/m², 18.0 mg/m², 18.5 mg/m², 19.0 mg/m² and 19.5 mg/m², 20.0 mg/m², 20.5 mg/m², 21.0 mg/m², 22.5 mg/m², 23.0 mg/m², 23.5 mg/m², 24.0 mg/m², 24.5 mg/m², 25.0 mg/m², 25.5 mg/m², 26.0 mg/m², 26.5 mg/m², 27.0 mg/m², 27.5 mg/m², 28.0 mg/m², 28.5 mg/m², 29.0 mg/m², 29.5 mg/m², 30.0 mg/m², 30.5 mg/m², 31.0 mg/m², 31.5 mg/m², 32.0 mg/m², 32.5 mg/m², 33.0 mg/m², 33.5 mg/m², 34.0 mg/m², 34.5 mg/m², 35.0 mg/m², 35.5 mg/m², 36.0 mg/m², 36.5 mg/m², 37.0 mg/m², 37.5 mg/m², 38.0 mg/m², 38.5 mg/m², 39.0 mg/m², 39.5 mg/m², 40.0 mg/m², 40.5 mg/m², 41.0 mg/m², 41.5 mg/m², 42.0 mg/m², 42.5 mg/m², 43.0 mg/m², 43.5 mg/m², 44.0 mg/m², 44.5 mg/m², 45.0 mg/m², 45.5 mg/m², 46.0 mg/m², 46.5 mg/m², 47.0 mg/m², 47.5 mg/m², 48.0 mg/m², 48.5 mg/m², 49.0 mg/m², 49.5 mg/m², 50.0 mg/m², 50.5 mg/m², 51.0 mg/m², 51.5 mg/m², 52.0 mg/m², 52.5 mg/m², 53.0 mg/m², 53.5 mg/m², 54.0 mg/m², 54.5 mg/m², 55.0 mg/m², 55.5 mg/m², 56.0 mg/m², 56.5 mg/m², 57.0 mg/m², 57.5 mg/m², 58.0 mg/m², 58.5 mg/m², 59.0 mg/m², 59.5 mg/m², 60.0 mg/m², 60.5 mg/m², 61.0 mg/m², 61.5 mg/m², 62.0 mg/m², 62.5 mg/m², 63.0 mg/m², 63.5 mg/m², 64.0 mg/m², 64.5 mg/m², 65.0 mg/m², 65.5 mg/m², 66.0 mg/m², 66.5 mg/m², 67.0 mg/m², 67.5 mg/m², 68.0 mg/m², 68.5 mg/m², 69.0 mg/m², 69.5 mg/m², 70.0 mg/m², 70.5 mg/m², 71.0 mg/m², 71.5 mg/m², 72.0 mg/m², 72.5 mg/m², 73.0 mg/m², 73.5 mg/m², 74.0 mg/m², 74.5 mg/m², 75.0 mg/m², 75.5 mg/m², 76.0 mg/m², 76.5 mg/m², 77.0 mg/m², 77.5 mg/m², 78.0 mg/m², 78.5 mg/m², 79.0 mg/m², 79.5 mg/m², 80.0 mg/m², 80.5 mg/m², 81.0 mg/m², 81.5 mg/m², 82.0 mg/m², 82.5 mg/m², 83.0 mg/m², 83.5 mg/m², 84.0 mg/m², 84.5 mg/m², 85.0 mg/m², 85.5 mg/m², 86.0 mg/m², 86.5 mg/m², 87.0 mg/m², 87.5 mg/m², 88.0 mg/m², 88.5 mg/m², 89.0 mg/m², 89.5 mg/m², 90.0 mg/m², 90.5 mg/m², 91.0 mg/m², 91.5 mg/m², 92.0 mg/m², 92.5 mg/m², 93.0 mg/m², 93.5 mg/m², 94.0 mg/m², 94.5 mg/m², 95.0 mg/m², 95.5 mg/m², 96.0 mg/m², 96.5 mg/m², 97.0 mg/m², 97.5 mg/m², 98.0 mg/m², 98.5 mg/m², 99.0 mg/m², or 99.5 mg/m². The total dose may be administered in single or divided doses and may, at the physician's discretion, fall outside of the typical range given herein.

The folate-imaging agent conjugates and compounds described herein may contain one or more chiral centers, or may otherwise be capable of existing as multiple stereoisomers. Accordingly, it is to be understood that the present invention includes pure stereoisomers as well as mixtures of stereoisomers, such as enantiomers, diastereomers, and enantiomerically or diastereomerically enriched mixtures. The folate-imaging agent conjugates and compounds described herein may be capable of existing as geometric isomers. Accordingly, it is to be understood that the present invention includes pure geometric isomers or mixtures of geometric isomers.

It is appreciated that the folate-imaging agent conjugates and compounds described herein may exist in unsolvated forms as well as solvated forms, including hydrated forms. In general, the solvated forms are equivalent to unsolvated forms and are encompassed within the scope of the present invention. The folate-imaging agent conjugates and compounds described herein may exist in multiple crystalline or amorphous forms. In general, all physical forms are equivalent for the uses contemplated by the present invention and are intended to be within the scope of the present invention.

In another embodiment, compositions and/or dosage forms for administration of Compound I are prepared from Compound I with a purity of at least about 90%, or about 95%, or about 96%, or about 97%, or about 98%, or about 99%, or about 99.5%. In another embodiment, compositions and or dosage forms for administration of Compound I are prepared from Compound I with a purity of at least 90%, or at least 95%, or at least 96%, or at least 97%, or at least 98%, or at least 99%, or at least 99.5%.

In another embodiment, compositions and/or dosage forms for administration of the folate-imaging agent conjugate are prepared from the folate-imaging agent conjugate with a purity of at least about 90%, or about 95%, or about 96%, or about 97%, or about 98%, or about 99%, or about 99.5%. In another embodiment, compositions and or dosage forms for administration of the folate-imaging agent conjugate are prepared from the folate-imaging agent conjugate with a purity of at least 90%, or at least 95%, or at least 97%, or at least 98%, or at least 99%, or at least 99.5%.

In another embodiment, compositions and/or dosage forms for administration of radiolabeled folate-imaging agent conjugate are prepared from the folate-imaging agent conjugate with a radiochemical purity of at least about 90%, or about 95%, or about 96%, or about 97%, or about 98%, or about 99%, or about 99.5%. In another embodiment, compositions and or dosage forms for administration of the folate-imaging agent conjugate are prepared from the folate-imaging agent conjugate with a purity of at least 90%, or at least 95%, or at least 96%, or at least 97%, or at least 98%, or at least 99%, or at least 99.5%.

As used herein, purity determinations may be based on weight percentage, mole percentage, and the like. In addition, purity determinations may be based on the absence or substantial absence of certain predetermined components, such as, but not limited to, folic acid, disulfide containing components not containing a vinca drug, oxidation products, disulfide components not containing a folate, and the like. It is also to be understood that purity determinations are applicable to solutions of the compounds and folate-imaging agent conjugates purified by the methods described herein. In those instances, purity measurements, including weight percentage and mole percentage measurements, are related to the components of the solution exclusive of the solvent.

The purity of Compound I or the folate-imaging agent conjugates described herein may be measured using any conventional technique, including various chromatography or spectroscopic techniques, such as high pressure or high performance liquid chromatography (HPLC), nuclear magnetic resonance spectroscopy, TLC, UV absorbance spectroscopy, fluorescence spectroscopy, and the like.

In another embodiment, the compound or folate-imaging agent conjugate described herein is provided in a sterile container or package.

In one aspect, a clinical benefit of the patient to treatment with Compound I can be characterized utilizing Response Evaluation Criteria in Solid Tumors (RECIST) criteria. Illustratively, the criteria have been adapted from the original WHO Handbook (3), taking into account the measurement of the longest diameter for all target lesions: complete response, (CR)—the disappearance of all target lesions; partial response (PR)—at least a 30% decrease in the sum of the longest diameter of target lesions, taking as reference the baseline sum longest diameter; stable disease (SD)—neither sufficient shrinkage to qualify for partial response nor sufficient increase to qualify for progressive disease, taking as reference the smallest sum longest diameter since the treatment started; progressive disease (PD)—at least a 20% increase in the sum of the longest diameter of target lesions, taking as reference the smallest sum longest diameter recorded since the treatment started or the appearance of one or more new lesions. In another aspect overall disease response rate (ORR) is a clinical benefit and is calculated as the percent of patients who achieve a best response of CR or PR. Overall disease control rate (DCR) can be another clinical benefit and is calculated as the percent of patients who achieve a best response of CR, PR, or SD.

In one illustrative example overall survival is the time to death for a given patient defined as the number of days from the first day the patient received protocol treatment (C1D1) to the date of the patient's death. All events of death can be included, regardless of whether the event occurred while the patient was still taking the study drug or after the patient discontinued the study drug. If a patient has not died, then the data can be censored at the last study visit, or the last contact date, or the date the patient was last known to be alive, whichever is last.

Alternatively, a clinical benefit of the patient as a result of treatment with Compound I can be characterized as inhibition of tumor growth which can be identified in a patient through, for example, follow-up imaging of the patient's cancer after treatment with Compound I. For example, inhibition of tumor growth can be characterized by measuring the size of tumors in a patient after administration of Compound I according to any of the imaging techniques described herein, where the inhibition of tumor growth is indicated by a stable tumor size, or by a reduction in tumor size. It will be appreciated that the identification of inhibition of tumor growth can be accomplished using a variety of techniques, and is not limited to the imaging methods described herein (e.g CT, MRI, PET imaging, SPECT imaging or chest x-ray)

In one embodiment, a method is provided of determining whether Compound I is indicated for the treatment of a patient with cancer, the method comprising the step of determining the folate-receptor status in a patient with cancer wherein Compound I is indicated for the treatment of the patient if the folate-receptor status of the patient is positive.

In one embodiment, a method is provided of assessing whether Compound I is indicated for the treatment of a patient with one of the cancers described herein. The method comprises the steps of visually determining folate receptor status in the patient wherein folate receptor status is based on a measurement of the percentage of evaluable tumors that are folate receptor positive in the patient, and wherein the Compound I is indicated for the treatment of the patient when the folate receptor status of the patient is positive. In an illustrative embodiment, positive folate receptor status means that the percentage of evaluable tumors in the patient that are folate receptor positive is about 100%. In other illustrative aspects, positive folate receptor status means that the percentage of evaluable tumors in the patient that are folate receptor positive is about 90%, about 80%, about 70%, about 60%, about 50%, at least about 50%, at least about 55%, at least about 60%, at least about 65%, at least about 70%, at least about 75%, at least about 80%, at least about 85%, at least about 90%, or at least about 95%. In another embodiment, positive folate receptor status means that the percentage of evaluable tumors in the patient that are folate receptor positive is 55%, 60%, 65%, 70%, 75%, 80%, 85%, 90%, 95% or 100%.

In this visual assessment embodiment, lesions are evaluated visually by examination of an image (e.g., a SPECT image) to determine if the patient has a threshold level of functionally active folate receptors indicative of a clinical benefit to the patient. In one aspect, lesions (i.e., tumors) for analysis in each patient are selected by a radiologist according to RECIST (v1.1) criteria. Subsequently, a nuclear medicine physician (i.e. reader) assesses the uptake of the folate-imaging agent conjugates described herein for each evaluable target lesion visually, and classifies the uptake as “positive” (marked uptake/mild uptake) or “negative” (no uptake). In one illustrative example the folate imaging agent conjugate is ^{99m}Tc-etarfolatide. The term “no uptake” means that visual inspection of the target lesion compared with the nearby tissue indicates that uptake of the folate-imaging agent conjugate in the target lesion and nearby tissue are not distinguishable. The term “mild uptake” means that visual inspection of the target lesion compared with the nearby tissue indicates that uptake of the folate-imaging agent conjugate in the target lesion and in nearby tissue are distinguishable. The term “marked uptake” means that visual inspection of the target lesion compared with the nearby tissue indicates that uptake of the folate-imaging agent conjugate in the target lesion and in nearby tissue are clearly distinguishable.

In these embodiments, lesions can be evaluable or non-evaluable. In one embodiment, lesions less than 1.5 cm in longest dimension (LD) are considered “non-evaluable” unless the nuclear medicine reader identified them as having unequivocal uptake of the folate-imaging agent conjugate, in which case they are characterized as “positive.” Moreover, certain organs (e.g., liver, spleen, bladder, and kidney) have an inherently high uptake of folate-imaging agent conjugates. Target lesions located in these organs are considered “non-evaluable.” In another embodiment, non-evaluable lesions fit one of the following criteria: 1) defined as “not imaged” upon target lesion evaluation, 2) defined as negative for folate-imaging agent conjugate uptake and less than 15 mm in diameter or 3) lesion located in the liver, kidney/adrenal gland, spleen, or bladder. In one embodiment, evaluable lesions fit one of the following criteria: 1) defined as positive for uptake as described above, or 2) defined as negative for uptake and greater than or equal to 15 mm in diameter.

In one embodiment, the percentage of lesions that are positive in each patient is calculated as follows: % positive lesions=(number of positive lesions/number of positive lesions+number of negative lesions+number of non-evaluable lesions).

In the above-described embodiments, if a patient is in the group with positive folate-receptor status, a clinical benefit of Compound I treatment is indicated. In one embodiment, the clinical benefit to the patient can be overall survival of the patient, ability to receive four or more cycles of therapy with Compound I, inhibition of tumor growth, stable disease, a partial response of the patient to therapy, a complete response of the patient to therapy, disease control (i.e., the best result obtained is a complete response, a partial response, or stable disease), and/or overall disease response (i.e., the best result obtained is a complete response or a partial response). In one illustrative example, the clinical benefit for a patient being treated for pleural mesothelioma or adenocarcinoma (e.g. adenocarcinoma of the gastroesophageal junction) is stable disease.

In any of the imaging methods described herein for detecting or determining, unlabeled folic acid, or a pharmaceutically acceptable salt thereof, can be administered to the patient before the folate-imaging agent conjugate, or a pharmaceutically acceptable salt thereof, is administered to the patient.

In another embodiment, the methods described herein include the following examples. The examples further illustrate additional features of the various embodiments of the invention described herein. However, it is to be understood that the examples are illustrative and are not to be construed as limiting other embodiments of the invention described herein. In addition, it is appreciated that other variations of the examples are included in the various embodiments of the invention described herein.

### EXAMPLES

### 1. Preparation of Compound I:

Compound I was prepared according to the methods described in International Patent Publication No. WO2014062697, incorporated herein by reference. See for example pages 76 to 91 of WO2014062697.

### a. Example. EC1426 is Prepared According to the Following Process

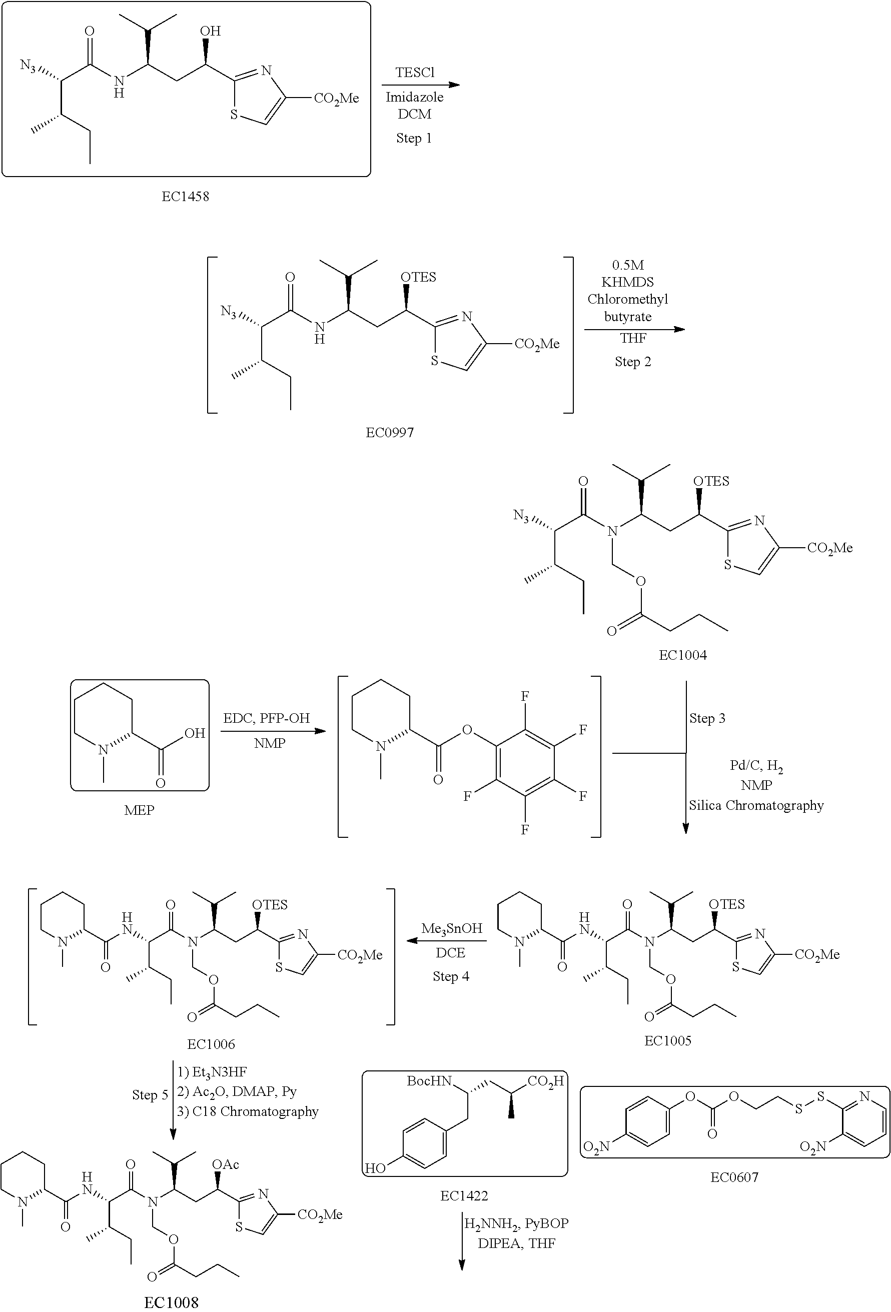

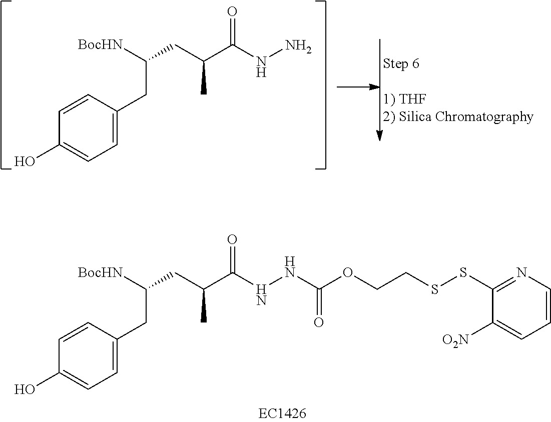

### b. Example. EC1456 is Prepared According to the Following Process

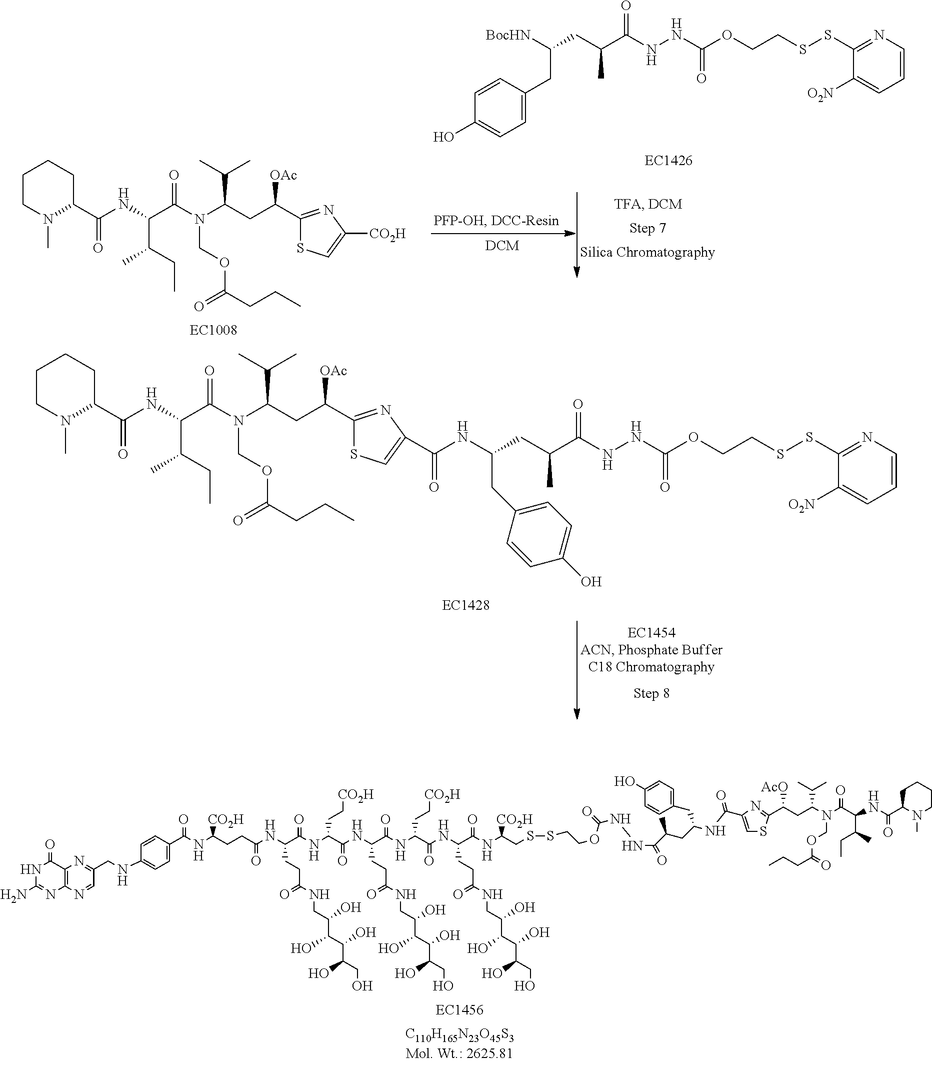

### c. Example. N¹⁰-TFA Protected EC1454 is Prepared According to the Following Process

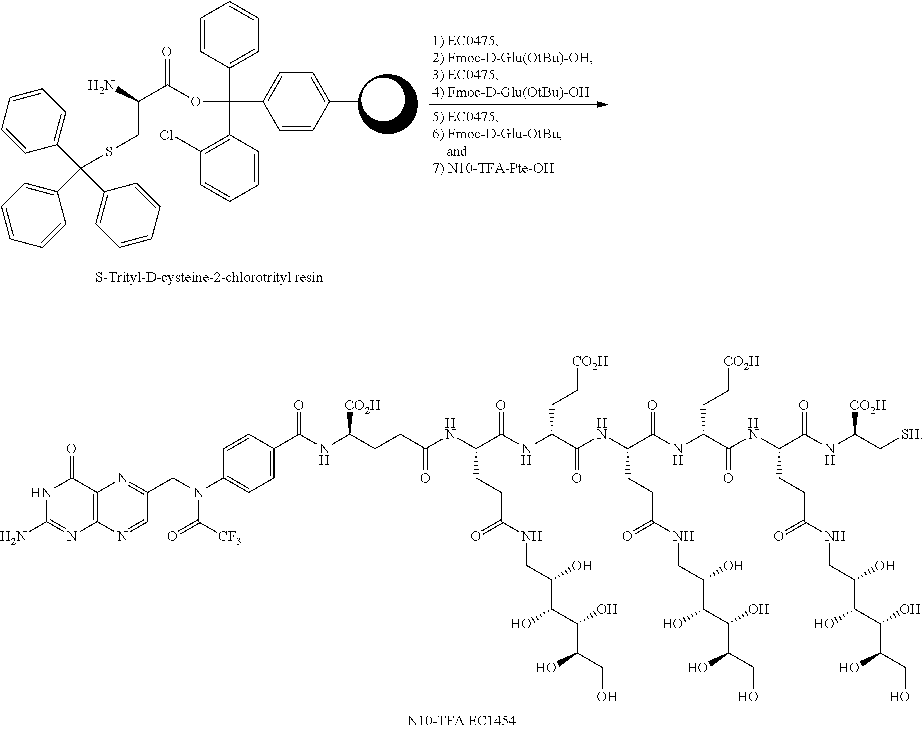

### d. Example. EC1454 is Prepared According to the Following Process

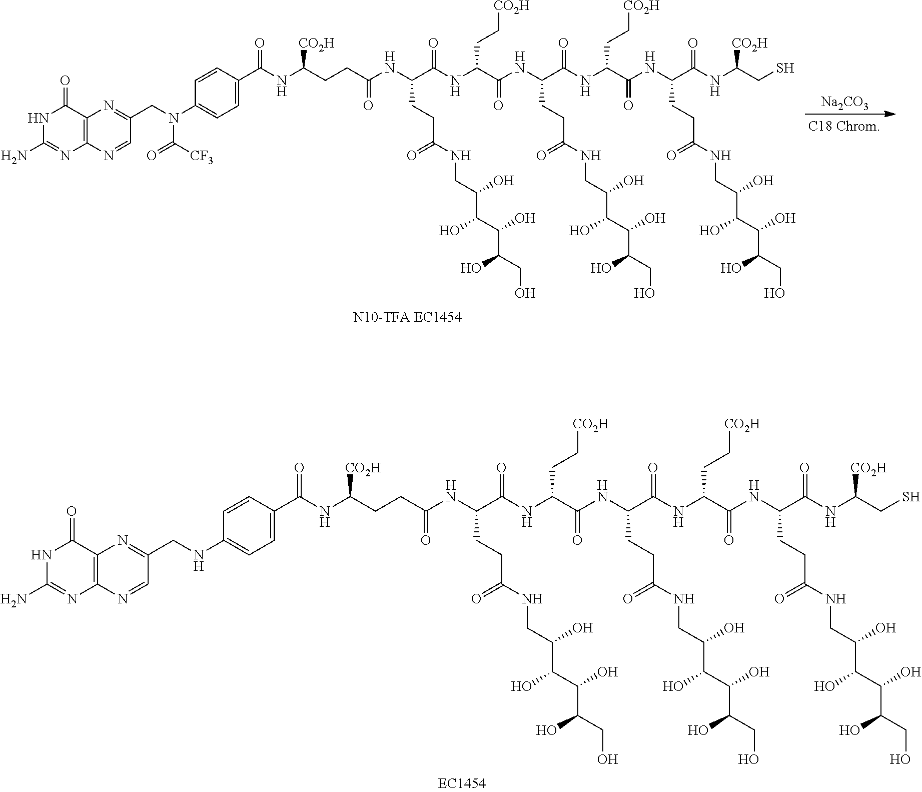

EC1454: MS (ESI, [M+2H]²⁺)=840.90, [M+H]⁺=1681.3. Partial 1H-NMR (DMSO) δ(ppm): 8.6(s), 7.6(d), 6.6(d), 4.45(s), 4.35(t), 4.15-4.3(m), 3.3-3.6(m), 3.25(m), 3.0(m), 2.7-2.9(m), 2-2.3(m), 1.6-2(m).

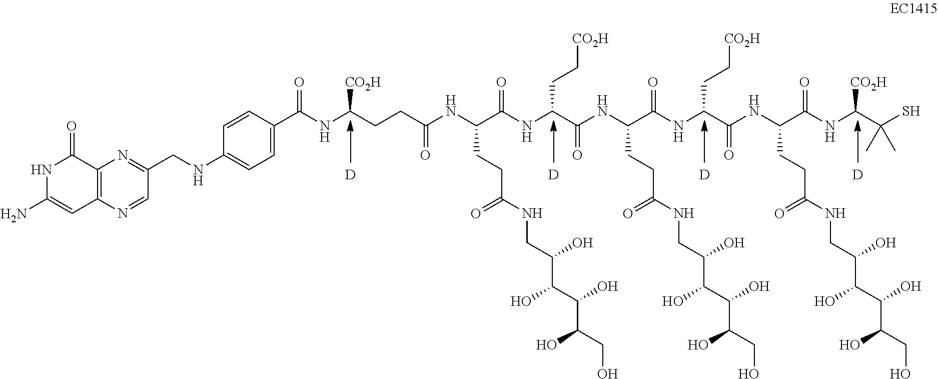

EC1415: [M+H]⁺=1709.69, [M+2H]²⁺=855.22. Partial ¹H NMR (D2O, 300 MHz) δ(ppm): 8.6 (s, 1H), 7.45 (d, 2H), 6.5 (d, 2H), 4.5 (s, 2H), 4.3-4.1 (m, 6H), 3.95 (t, 1H), 3.8-3.4 (m, 19H), 3.4-2.95 (m, 7H), 2.4-1.7 (m, 26H), 1.6 (m, 1H), 1.25 (s, 2H), 1.05 (s, 3H).

### e. Example. EC1004 ss Prepared According to the Following Process

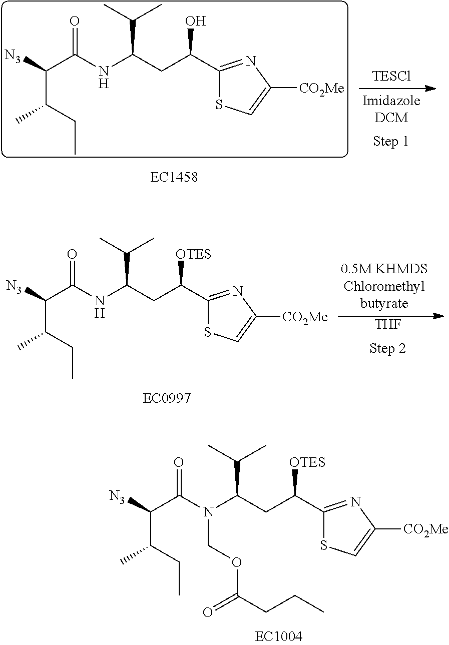

Into a round bottomed flask equipped with magnetic stir bar and temperature probe dipeptide EC1458, imidazole, and methylene chloride is added. Once all the solids have dissolved, the solution is cooled using an ice bath. Chlorotriethylsilane (TESCl) is added drop wise and the ice bath is removed. The reaction is monitored for completion. A second portion of chlorotriethylsilane and/or imidazole is added if necessary. The imidazole HCl salt is removed by filtration and methylene chloride is added. The organics are washed with a saturated solution of sodium chloride (brine), the aqueous layer is back extracted once with methylene chloride, and the combined organic layers are washed with brine. The organic layer is dried over sodium sulfate and concentrated on a rotary evaporator. The residue is dissolved in tetrahydrofuran (THF) and cooled to approximately −45° C. A solution of potassium bis(trimethylsilyl)amide (KHMDS) in toluene is added drop wise. With stiffing, chloromethyl butyrate is added and the reaction is monitored. The reaction is quenched with methanol and then ethyl acetate and brine are added. The aqueous layer is discarded and the organics are washed once with brine. The organic layer is concentrated on a rotary evaporator and the oily residue is passed through a short plug of silica gel. The plug is washed with a 20% solution of ethyl acetate in petroleum ether. The combined organics are concentrated on a rotary evaporator until distillation ceases. The crude EC1004 oil is analyzed by LC and NMR and stored in a freezer until use.

### f. Example. EC1005 is Prepared According to the Following Process

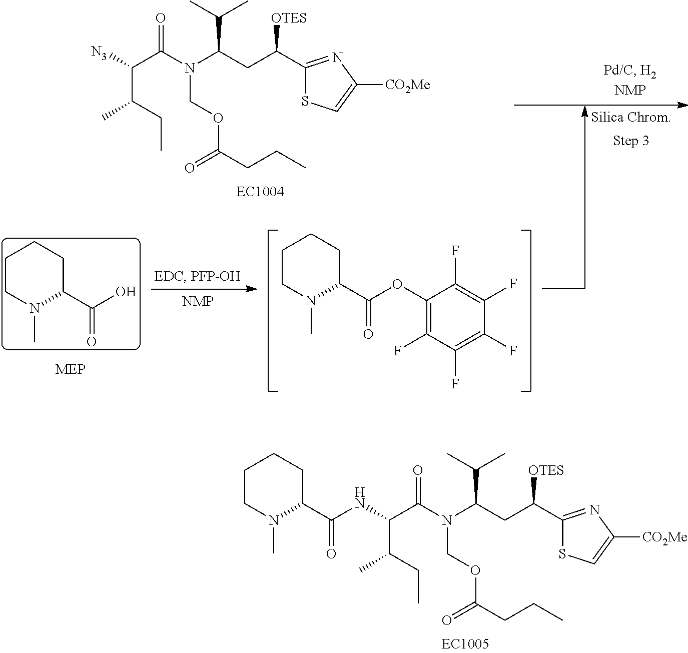

Into an appropriately sized hydrogenation flask place R-N-methyl pipecolinate (MEP), pentafluorophenol, N-methyl pyrrolidinone (NMP), and ethyl dimethylaminopropyl carbodiimide (EDC). The mixture is stirred for at least 16 h. EC1004 dissolved in N-methyl pyrrolidinone (NMP) and 10 wt % Pd/C are added. The reaction mixture is stirred/shaken under hydrogen pressure until the reaction is complete by LC analysis. The Pd/C is removed by filtration through celite. The celite is washed with ethyl acetate and the combined organics are washed three times with a 1% sodium bicarbonate/10% sodium chloride solution. The organic layer is dried over sodium sulfate and concentrated on a rotary evaporator. The residue is dissolved in DCM and purified by silica gel chromatography using ethyl acetate and petroleum ether as eluents. Fractions are collected, checked for purity, combined and dried on a rotary evaporator. The EC1005 oil is assayed by LC and stored in a freezer until use.

### g. Example. EC1008 is Prepared According to the Following Process

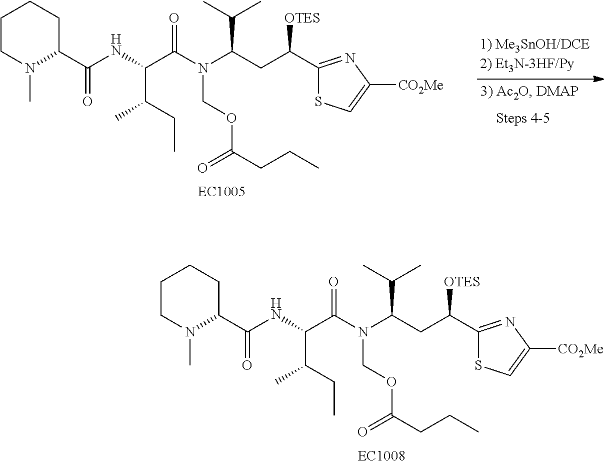

EC1005 is dissolved in 1,2-dichloroethane (DCE) and trimethyltin hydroxide is added. The reaction mixture is heated and reaction is monitored by LC. On completion, the mixture is cooled with an ice bath and filtered. The solids are then washed with DCE. The organic layer is washed once with water and dried over sodium sulfate. The solution is concentrated on a rotary evaporator and the residue dissolved in tetrahydrofuran (THF). Triethylamine trihydrofluoride is added and the mixture stirred while monitoring with LC. Pyridine, dimethylaminopyridine (DMAP), and acetic anhydride are added. The reaction is stirred and monitored by LC. The reaction mixture is concentrated to a residue and the product is purified by C18 column chromatography with acetonitrile and water as eluents. Product fractions are collected, concentrated, and lyophilized to yield a white to off-white powder.

### h. Example. EC1426 is Prepared According to the Following Process

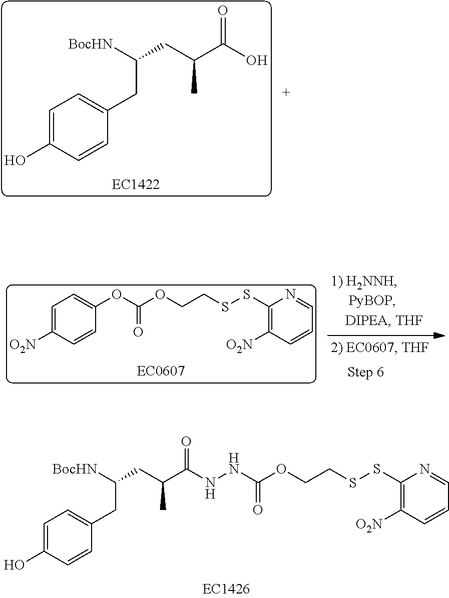 [SMILES: C[C@@H](C[C@H](Cc1ccc(O)cc1)NC(=O)OC(C)(C)C)C(=O)O ; O=C(OCCSSc1ncccc1[N+](=O)[O-])Oc1ccc([N+](=O)[O-])cc1 ; C[C@@H](C[C@H](Cc1ccc(O)cc1)NC(=O)OC(C)(C)C)C(=O)NNC(=O)OCCSSc1ncccc1[N+](=O)[O-]]

EC1422 is dissolved in tetrahydrofuran (THF) and (Benzotriazol-1-yloxy)tripyrrolidinophosphonium hexafluorophosphate (PyBop) and diisopropylethylamine (DIPEA) are added. Once all the solids have dissolved hydrazine is added and the reaction is stirred and monitored for completion. EC0607 is added and the mixture stirred and monitored for completion by LC. Ethyl acetate is added and the organics are washed once with saturated ammonium chloride, twice with saturated sodium bicarbonate, and once with saturated sodium chloride. The organics are dried over sodium sulfate and concentrated on a rotary evaporator. The crude EC1426 is purified by silica column chromatography with dichloromethane and methanol as eluents. Fractions are collected and the combined product fractions are concentrated on a rotary evaporator to yield a yellow solid.

### i. Example. EC1428 is Prepared According to the Following Process

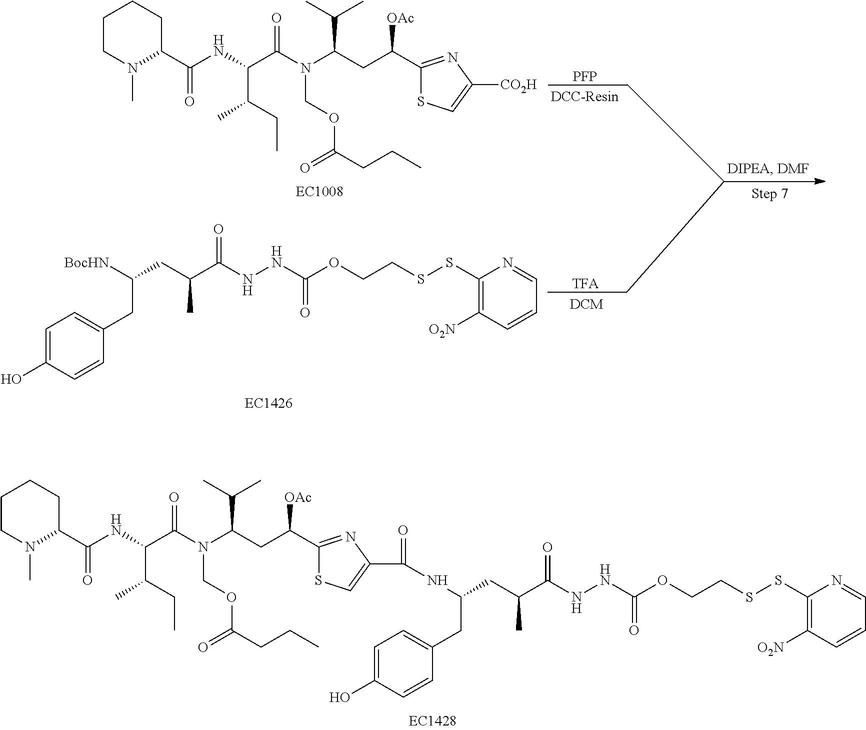

EC1008 is dissolved in dichloromethane and pentafluorophenol dissolved in DCM along with N-cyclohexylcarbodiimide,N′-methyl polystyrene (DCC-resin) are added. The mixture is stirred and reaction completion is monitord by LC. The mixture is filtered to remove the resin and the organic layer is concentrated on a rotary evaporator to yield activated EC1008. In a separate flask, EC1426 is dissolved in dichloromethane and trifluoroacetic acid is added. The reaction mixture is stirred and monitored for completion by LC. The reaction mixture is concentrated on a rotary evaporator to yield deprotected EC1426. The activated EC1008 is dissolved in DMF and diisopropylethylamine (DIPEA) is added. The deprotected EC1426 is dissolved in DMF and added to the reaction mixture. The reaction is stirred and monitored for completion by LC. Ethyl acetate is added and the organics are washed three times with saturated aqueous sodium chloride. The organic layer is dried over sodium sulfate and the volatiles removed by rotary evaporation. The crude EC1428 is purified by silica column chromatography using dichloromethane and methanol as eluents. Fractions are collected, checked for purity, and the combined product fractions are concentrated by rotary evaporation to yield a yellow solid. The EC1428 is stored in a freezer.

### j. Example. Illustrative Tubulysins are as Follows

100a-c

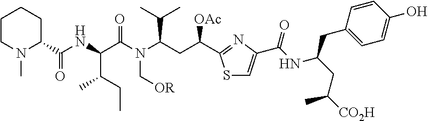

| Compound | 100a | 100b | 100c | Tub B |
|---|---|---|---|---|
| R | allyl | n-butyl | n-pentyl | |
| IC50 | 1.2 | 0.7 | 0.8 | 1.2 |
| on FR+ KB cell (nM) | | | | |

### k. Example. EC1454 is Prepared According to the Following Process

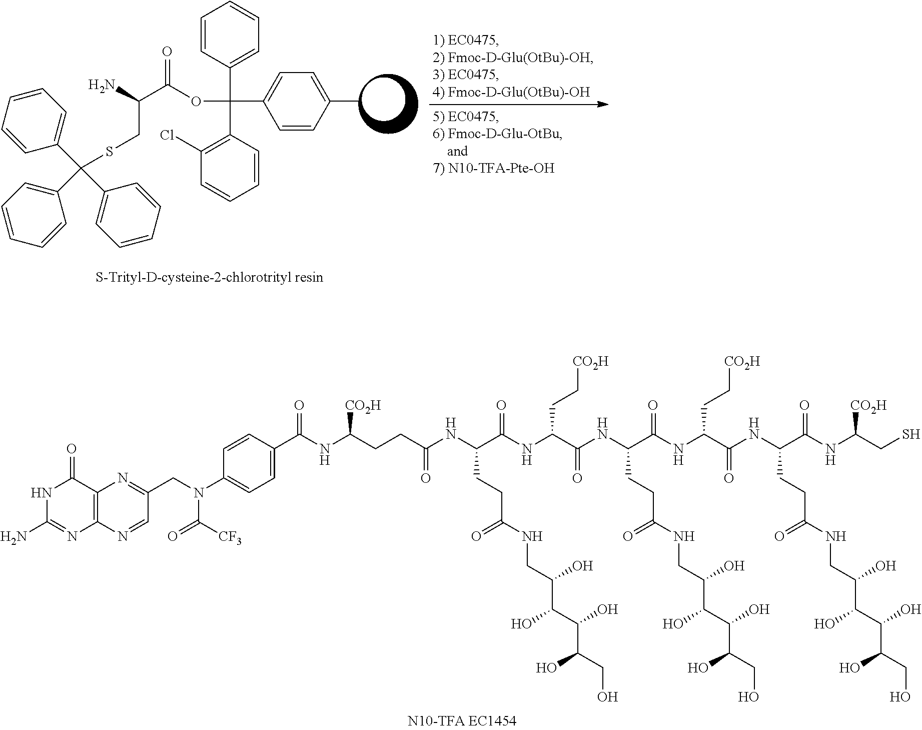

The solid phase synthesis of N¹⁰-TFA protected EC1454 starts with resin bound trityl protected D-cysteine. The resin is suspended in dimethylformamide (DMF) and washed twice with DMF. EC0475 (glucamine modified L-glutamic acid), (Benzotriazol-1-yloxy)tripyrrolidinophosphonium hexafluorophosphate (PyBOP), and diisopropylethylamine (DIPEA) are added to reaction mixture. After at least 1 hour, a Kaiser test is performed to ensure the coupling is complete. The resin is washed three times with DMF, three times with IPA, and three times with DMF. The resin is slowly washed three times with piperidine in DMF, three times with DMF, and three times with IPA. A Kaiser test is performed to confirm deprotection. The resin is washed three times with DMF and the next amino acid in the sequence is coupled following the same process. Monomers are coupled in the following order: 1) EC0475, 2) Fmoc-D-Glu(OtBu)-OH, 3) EC0475, 4) Fmoc-D-Glu(OtBu)-OH, 5) EC0475, 6) Fmoc-D-Glu-OtBu, and 7) N¹⁰-TFA-Pte-OH.

Once the final coupling is complete, the resin is washed three times with methanol and dried by passing argon through the resin at room temperature. The dried resin is suspended in a mixture of TFA, water, ethanedithiol, and triisopropylsilane. After 1 hour the resin is removed by filtration and washed with TFA. The product is precipitated by addition to cold ethyl ether, filtered, and washed with ether. The solids are dried under vacuum at room temperature and stored in a freezer.

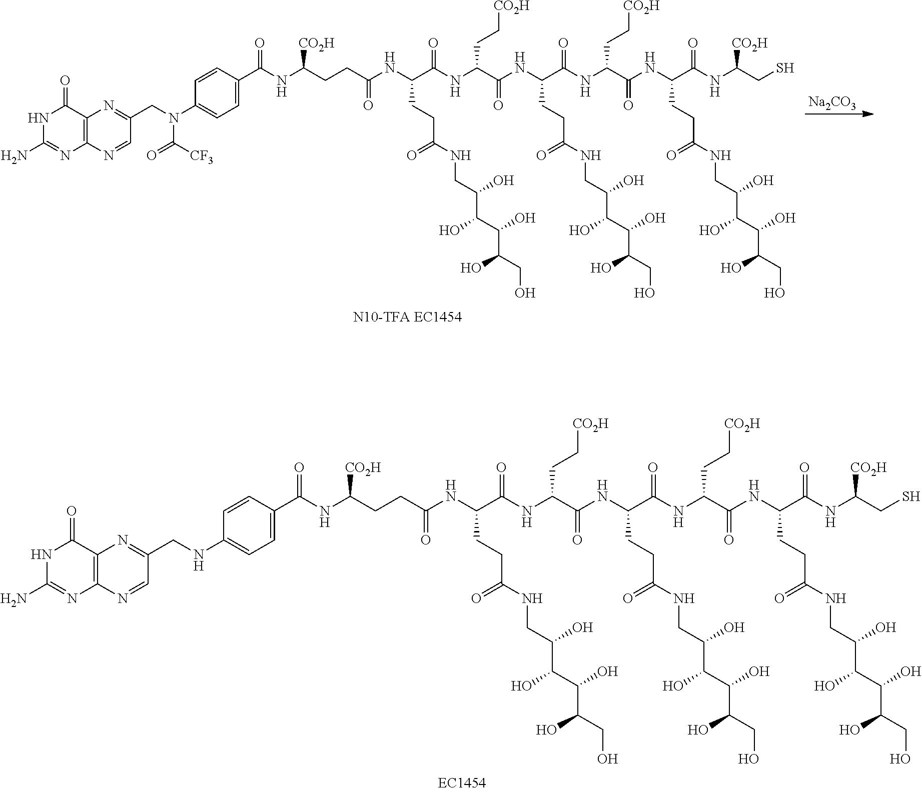

N¹⁰-TFA EC1454 is dissolved in argon sparged water. Sodium carbonate (1M in water, argon sparged) is added to achieve a pH of 9.4-10.1. The reaction mixture is stirred for at least 20 minutes. Once the reaction is complete as determined by LC, it is quenched by adjusting the pH to 1.9-2.3 with 2M HCl. The product is purified by C18 column chromatography using acetonitrile and pH 5 ammonium acetate buffer as eluents. Fractions are collected and checked for purity by HPLC. The combined product fractions are concentrated on a rotary evaporator and then lyophilized to yield EC1454 as a yellow solid. MS (ESI, [M+2H]²⁺)=840.90, [M+H1]⁺=1681.3. Selected 1H-NMR (DMSO, 300 MHz) δ(ppm): 8.6(s), 7.6(d), 6.6(d), 4.45(s), 4.35(t), 4.15-4.3(m), 3.3-3.6(m), 3.25(m), 3.0(m), 2.7-2.9(m), 2-2.3(m), 1.6-2(m). The product is stored at −20° C.

### l. Example. EC1456 is Prepared According to the Following Process

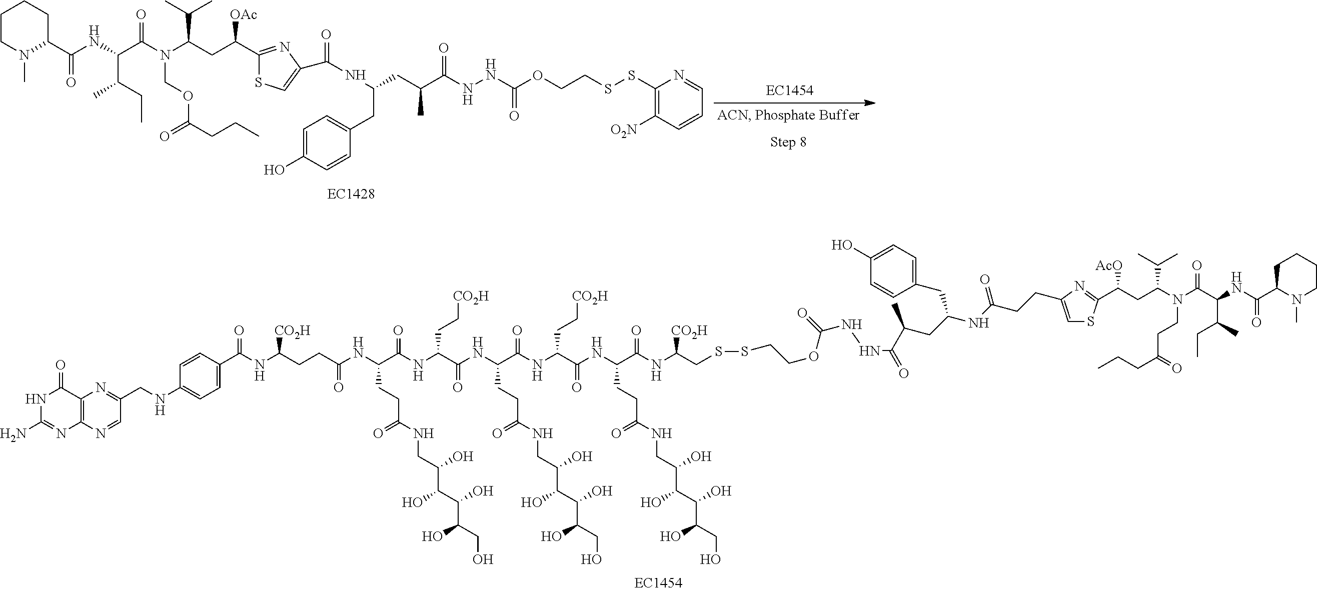

EC1428 is dissolved in acetonitrile and a solution of EC1454 in pH 7.4 Sodium phosphate buffer is added. The solutions are sparged with argon before and after addition. The reaction mixture is stirred for at least 15 minutes and then checked for completion. The desired product is purified by C18 column chromatography using acetonitrile and pH 7.4 phosphate buffer as eluents. The product fractions are collected, checked for purity, combined and concentrated by ultra-filtration to yield an aqueous solution that is 10-20 mg/mL EC1456. The final product solution is sampled for assay and then stored in a freezer.

The positive electrospray mass spectrum of EC1456 was obtained on a high resolution Waters Acquity UPLC Xevo Gs-S QTOF mass spectrometer. The spectrum was obtained following separation of the major component on a UPLC inlet system, the resolving power was approximately 35,000. The accurate mass measurement of the M+H monoisotopic peak was 2625.0598, which is 1.1 ppm error difference from the theoretical value of 2625.0570 for an ion of formula C₁₁₀H₁₆₆N₂₃O₄₅S₃. The isotopic distribution is also consistent with that formula. Mass spectral features of the ES+ spectrum for EC1456

| Observed Ion | Interpretation |
|---|---|
| 2626.06 | ¹³C isotope of the (M + H)⁺ ion for the MW 2624 |
| | drug substance |
| 1313.54 | ¹³C isotope of the (M + 2H)⁺⁺ ion for the MW 2624 |
| | drug substance |
| 1150.43 | ¹³C isotope of the (M + 2H − 326)⁺⁺ fragment, |
| | corresponding to the cleavage of the peptide bond at the |
| | tertiary nitrogen and the loss of the butyric acid moiety. |
| 876.03 | ¹³C isotope of the (M + 3H)⁺⁺⁺ ion for the MW 2624 |
| | drug substance |
| 657.27 | ¹³C isotope of the (M + 4H)⁺⁺⁺⁺ ion for the MW 2624 |
| | drug substance |

A sample of ˜30 mg EC1456 was dissolved in 665 μL, of a 9:1 mixture of deuterated dimethylsulfoxide and deuterated water. The ¹H NMR spectrum was obtained at 500 MHz at 26 deg. C on an Agilent model DD2 spectrometer fitted with a 2 channel probe containing both broadband and proton observe coils. The ¹³C NMR spectrum was obtained at 125 MHz on the same instrument under identical conditions. All spectra were referenced to the DMSO solvent residual signals at 2.5 ppm (¹H) and 39.50 ppm (¹³C).

All spectral features are assigned for both NMR spectra in the tables below (¹H and ¹³C) using the atom numbering in the following figure, where the * symbols indicate the connection for the disulfide bond.

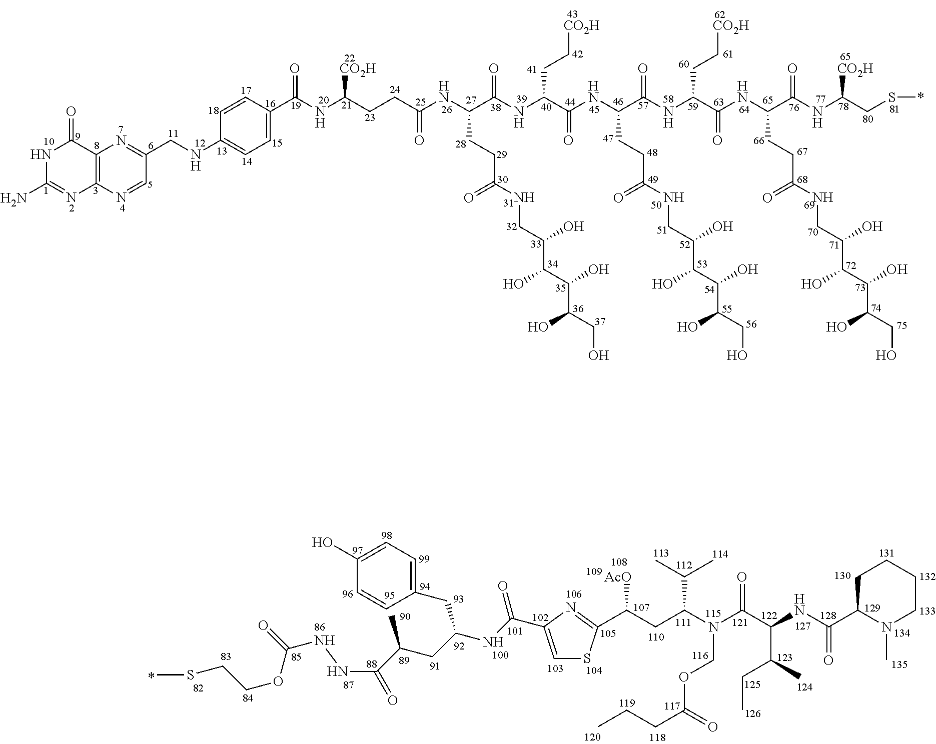 [SMILES: *SC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c1ccc(NCc2cnc3nc(N)[nH]c(=O)c3n2)cc1)C(=O)O)C(=O)O ; CCCC(=O)OCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)C[C@H](C)C(=O)NNC(=O)OCCSC)cs1)C(C)C]

Assignments were made on the basis of both 1D and 2D NMR experiments, including through bond H—H connectivity using the COSY and TCSY 2D experiments, through space H—H proximity using 2D NOESY, carbon multiplicity measurement using the 1D DEPT experiment and through bond C—H connectivity using the proton detected 2D experiments HSQC and HMBC. In most cases of overlap in the 1D spectra (different protons or carbons resonating at the same chemical shift) could be resolved in the 2D spectra, in these cases the tables reflect the chemical shifts measured from the 2D spectra but summed integrations for the group of co-resonating species. In some cases of 1D overlap (such as the nearly identical glutamic acid and glucamine subunits) there was also overlap in the 2D correlation spectra which precludes unambiguous assignment of single or multiple resonances between multiple atom numbers, in these cases there are multiple entries for chemical shift and/or atom number assignments in a single table row.

NH and OH protons were exchanged by the D₂O deuterium atoms and are mostly absent from the spectrum, except weak broad peaks in the 5-10 ppm region. The ¹H peaks in the spectrum that are not listed in the table include a broad HOD peak at 3.75 ppm, and a DMSO peak at 2.50 ppm. The HOD peak does not obscure any resonances, but elevates the integrations for nearby resonances at 4.2 and 3.4-3.7 ppm due to the broad baseline rise. The DMSO peak obscures the resonance for H129, which is not integrated for this reason. The ¹³C peaks in spectrum not listed in the table include the very large DMSO solvent at 39.50 ppm. The DMSO peak obscures both the signals from C91 and C93. The C116 peak is not observable in the ¹³C spectrum due to extensive broadening due to conformational changes around the nearby amide group. All three chemical shifts (C91, C93, C116) are visible in and measured in the proton detected 2D correlation spectra.

Proton NMR assignments for EC1456

| Proton | | |
|---|---|---|
| Chemical Shift (ppm) | Assignment | # protons |
| 8.61 | 5 | 1 |
| 8.16 | 103 | 1 |
| 7.58 | 15, 17 | 2 |
| 6.96 | 95, 99 | 2 |
| 6.62 | 14, 18 | 4 |
| 6.59 | 96, 98 | |
| 6.18 | 116 Ha | 1 |
| 5.7 | 107 | 1 |
| 5.24 | 116 Hb | 1 |
| 4.47 | 11 | 2 |
| 4.39 | 111, 122 | 2 |
| 4.21 | 78 | 10 |
| 4.21 | 65 | |
| 4.18 | 84 | |
| 4.15 | 46 | |
| 4.15 | 59 | |
| 4.13 | 21 | |
| 4.13 | 40 | |
| 4.09 | 27 | |
| 4.09 | 92 | |
| 3.61 | 33, 52, 71 | 3 |
| 3.56 | 34, 53, 72 | 6 |
| 3.54 | 37Ha, 56Ha, 75Ha | |
| 3.46 | 36, 55, 74 | 3 |
| 3.4 | 35, 54, 73 | 6 |
| 3.38 | 37Hb, 56Hb, 75Hb | |
| 3.21 | 80Ha, 32Ha, 51 Ha, | 4 |
| | 70 Ha | |
| 3.05 | 32Hb, 51Hb, 70Hb | 3 |
| 2.93 | 80 Hb | 3 |
| 2.91 | 83 | |
| 2.8 | 133Ha | 1 |
| 2.68 | 93 | 2 |
| 2.49 (see text) | 129 | 1 |
| 2.35 | 89 | 2 |
| 2.33 | 110Ha | |
| 2.8 | 133Hb | 37 |
| 2.17 | 118 | |
| 2.14-2.08 | 24, 29, 42, 48, 61, 67 | |
| 2.09 | 110Hb | |
| 2.08 | 109 | |
| 2.02 | 135 | |
| 1.97-1.70 | 28, 41, 47, 60, 66 | |
| 1.92 | 23Ha | |
| 1.88 | 123 | |
| 1.8 | 91Ha | |
| 1.79 | 23Hb | |
| 1.77 | 112 | |
| 1.6 | 131Ha | 9 |
| 1.56 | 130Ha | |
| 1.5 | 132Ha | |
| 1.5 | 91Hb | |
| 1.45 | 125Ha | |
| 1.42 | 119 | |
| 1.4 | 132Hb | |
| 1.33 | 130Hb | |
| 1.14 | 131Hb | 2 |
| 1.07 | 125Hb | |
| 1 | 90 | 3 |
| 0.94 | 114 | 3 |
| 0.79 | 124 | 3 |
| 0.77 | 126 | 3 |
| 0.75 | 120 | 3 |
| 0.64 | 113 | 3 |

Carbon NMR assignments for EC1456

| Carbon | |
|---|---|
| Chemical shift | |
| (ppm) | Assignment |
| 176.77, 176.32 | 43, 62 |
| 175.74 | 88 |
| 175.42 | 22 |
| 174.75 | 121 |
| 173.87, 172.68, | 25, 38, 44, 57, 63 |
| 172.15, 171.94, | |
| 171.84 | |
| 173.43 | 79 |
| 173.3 | 128 |
| 172.79 (2x), | 30, 49, 68 |
| 172.72 | |
| 172.46 | 117 |
| 170.87 | 76 |
| 170.39 | 108 |
| 169.3 | 105 |
| 166.09 | 19 |
| 162.4 | 9 |
| 160.7 | 101 |
| 156.4 | 85 |
| 156.09 | 3 |
| 155.71 | 97 |
| 154.59 | 1 |
| 150.84 | 13 |
| 149.63 | 102 |
| 149.11 | 6 |
| 148.99 | 5 |
| 130.44 | 95, 99 |
| 128.99 | 15, 17 |
| 128.89 | 94 |
| 127.99 | 8 |
| 124.97 | 103 |
| 122.24 | 16 |
| 115.25 | 96, 98 |
| 111.86 | 14, 18 |
| 72.17 (3x) | 35, 54, 73 |
| 71.78, 71.74, 71.71 | 33, 52, 71 |
| 71.62, 71.59 (2x) | 36, 55, 74 |
| 69.65, 69.57 (2x) | 34, 53, 72 |
| 69.45 | 107 |
| 69.34 | 116 |
| 68.51 | 129 |
| 63.42 (3x) | 37, 56, 75 |
| 63.03 | 84 |
| 55.08 | 133 |
| 54.05 | 40 |
| 53.88 | 78 |
| 53.46 (2x) | 46, 59 |
| 53.33 | 27 |
| 52.96 (2x) | 122, 111 |
| 52.89 | 21 |
| 52.55 | 65 |
| 49.77 | 92 |
| 46.07 | 11 |
| 44.02 | 135 |
| 42.85 | 80 |
| 42.34 (2x), 42.29 | 32, 51, 70 |
| 39.52 | 93 |
| 38.95 | 91 |
| 37.43 | 83 |
| 35.95 | 118 |
| 35.43 | 123 |
| 35.38 | 89 |
| 34.86 | 110 |
| 32.56, 32.36, | 24, 29, 42, 48, 61, 67 |
| 32.16, 32.09 (2x), | |
| 31.81 | |
| 30.5 | 112 |
| 29.95 | 130 |
| 28.60, 28.04, 27.78 | 28, 41, 47, 60, 66 |
| (2x), 27.66 | |
| 27 | 23 |
| 25.01 | 132 |
| 24.43 | 125 |
| 23.04 | 131 |
| 20.86 | 109 |
| 20.56 | 114 |
| 19.64 | 113 |
| 18.36 | 90 |
| 18.04 | 119 |
| 15.64 | 124 |
| 13.72 | 120 |
| 10.28 | 126 |

The IR spectrum of EC1456 was acquired on a Nexus 6700® Fourier transform infrared (FT-IR) spectrophotometer (Thermo Nicolet) equipped with an Ever-Glo mid/far IR source, an extended range potassium bromide (KBr) beam splitter, and a deuterated triglycine sulfate (DTGS) detector. An attenuated total reflectance (ATR) accessory (Thunderdome™, Thermo Spectra-Tech), with a germanium (Ge) crystal was used for data acquisition. The spectrum represents 256 co-added scans collected at a spectral resolution of 4 cm-1. A background data set was acquired with a clean Ge crystal. A Log 1/R (R=reflectance) spectrum was acquired by taking a ratio of these two data sets against each other. Wavelength calibration was performed using polystyrene.

Infrared band assignments for EC1456 reference substance

| Characteristic Absorption(s) (cm⁻¹) | Functional Group |
|---|---|
| 1700-1500 (m, m) | Aromatic C═C Bending |
| 2950-2850 (m or s) | Alkyl C—H Stretch |
| ~3030 (v) | Aromatic C—H Stretch |
| 3550-3200 (broad, s) | Alcohol/Phenol O—H Stretch |
| 3700-3500 (m) | Amide C═O Stretch |

The ultraviolet spectrum EC1456 acquired on a Perkin-Elmer Lambda 25 UV/Vis spectrometer. The spectrum was recorded at 40.7 uM in 0.1M NaOH solvent on a 1 cm path-length cell at 25 deg. C. The local maxima at 366 nm, 288 nm and 243 nm are due primarily to the Pteroic acid, benzamide/phenol and thiazole-amide substructures, respectively, although the molecule contains dozens of chromaphores with overlapping absorption in the UV region.

### 2. Preclinical Studies:

a. Antitumor Activity on Paclitaxel Resistant Tumors

Four to six week-old female nu/nu mice (Harlan Sprague Dawley, Inc., Indianapolis, Ind.) were maintained on a standard 12 h light-dark cycle and fed ad libitum with folate-deficient chow (Harlan diet #TD00434, Harlan Teklad, Madison, Wis.) for the duration of the experiment. FR-positive paclitaxel resistant KB-PR10 cells were grown continuously as a monolayer, using folate-free RPMI medium (FFRPMI) containing 5% heat-inactivated fetal calf serum (HIFCS) at 37° C. in a 5% CO₂/95% air-humidified atmosphere with no antibiotics. KB-PR10 cells (1×10⁶ per nu/nu mouse) in 100 μL were injected in the subcutis of the dorsal medial area. Mice were divided into groups of five, and test articles were freshly prepared and injected through the lateral tail vein under sterile conditions in a volume of 200 μL of phosphate-buffered saline (PBS). Mice were treated with 20 mg/kg Paclitaxel, 2 μmol/kg EC145 (See WO2004/069159 for a description of EC145) or 1 μmol/kg Compound I on a three times a week for 2 weeks schedule when the tumors were approximately 100-145 mm³, 101-129 mm³ and 100-140 mm³ in volume, respectively. The mice in the control groups received no treatment. Growth of each s.c. tumor was followed by measuring the tumor three times per week during treatment and twice per week thereafter until a volume of 1500 mm³ was reached. Tumors were measured in two perpendicular directions using Vernier calipers, and their volumes were calculated as 0.5×L×W², where L=measurement of longest axis in mm and W=measurement of axis perpendicular to L in mm All in vivo studies were performed in accordance with the American Accreditation Association of Laboratory Animal Care guidelines.

### Results:

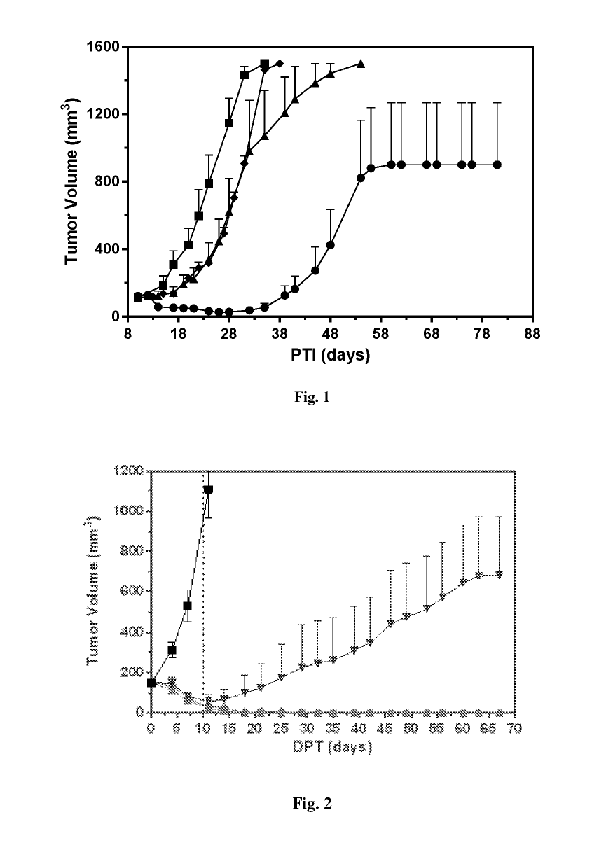

KB-PR10 cells are resistant to paclitaxel and express high levels of p-glycoprotein. As shown in FIG. 1, treatment with 20 mg/kg of paclitaxel (three times a week for two weeks) produced little to no anti-tumor activity with 0 partial responses. These tumors are also resistant to Compound I with a 2 μmol/kg dose (three times a week for two weeks) producing no-anti-tumor activity. However, EC1456 at 1 μmol/kg (three times a week for two weeks) produced good antitumor activity with 60% PRs and 40% cures.

b. Antitumor Activity in Huprime® NSCLC PDX Tumor Model

Female Balb/c nu/nu mice were fed ad libitum with folate-deficient chow (Harlan diet #TD01013) for the duration of the experiment. Primary human NSCLC models LU1147 or LU2505 fragments (2-4 mm in diameter) were inoculated subcutaneously at the right flank of each mouse. Mice were randomized into 6 experimental groups of 7 mice each as in the table below and test articles were injected through the lateral tail vein under sterile conditions in a volume of 200 μL of phosphate-buffered saline (PBS). These studies were performed at Crown Bioscience (Beijing) Inc., Ground Floor, Light Muller Building, Changping Sector of Zhongguancun Scientific Park, No. 21 Huoju Road, Changping District, Beijing, P.R. China.

| | | | | LU1147 | LU2505 |
|---|---|---|---|---|---|
| | | | Dosing | tumor size | tumor size |
| Group | Treatment | Dose level | Frequency | (mm³) | (mm³) |
| 1 | 0.9% | — | QW × 2 | 162.3 ± 20.9 | 151.0 ± 13.8 |
| | NaCl | | | | |
| 2 | EC1456 | 2 μmol/kg | BIW × 2 | 161.6 ± 15.5 | 151.1 ± 15.5 |
| 5 | EC1456 | 4 μmol/kg | QW × 2 | 179.6 ± 29.1 | 151.3 ± 15.5 |

Growth of each s.c. tumor was followed by measuring the tumor two times per week until a volume of 1200 mm³ was reached. Tumors were measured in two perpendicular directions using Vernier calipers, and their volumes were calculated as 0.5×L×W², where L=measurement of longest axis in mm and W=measurement of axis perpendicular to L in mm

### Results

Treatment with 15 mg/kg of Docetaxel (one dose) produced some anti-tumor activity in animals exhibiting stable disease with 2 PR's, 2 CR's and 2 cures. EC1456 at 2 μmol/kg (two times a week for two weeks) produced anti-tumor activity in mice bearing LU2505 tumors with cures in 7 of 7 animals. EC1456 at 4 μmol/kg (once a week for two weeks) produced anti-tumor activity in mice bearing LU2505 tumors with cures in 7 of 7 animals. See FIG. 2.

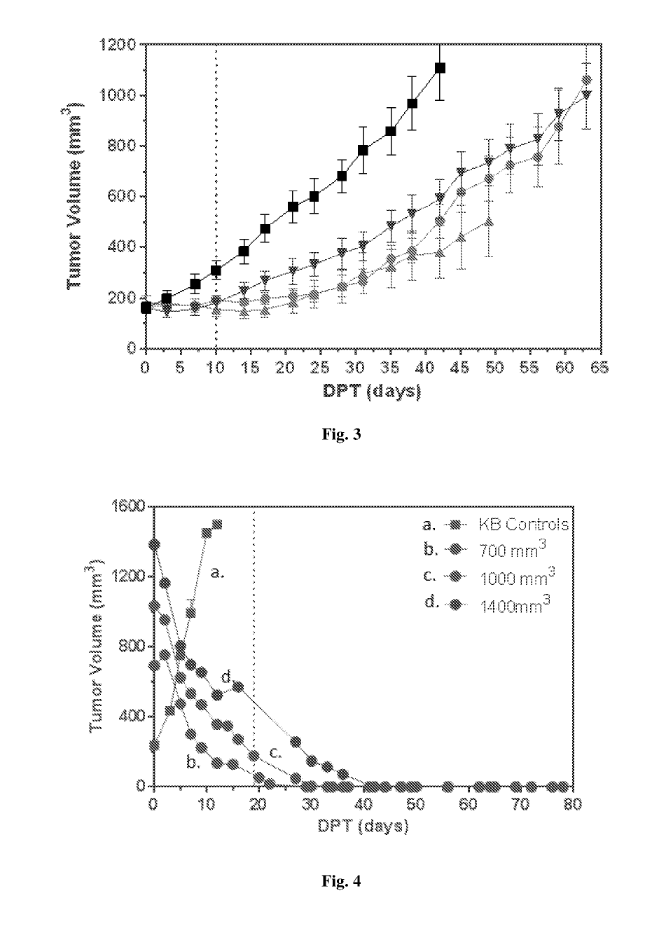

Treatment with 15 mg/kg of Docetaxel (one dose) produced minimal anti-tumor activity with 2 animals exhibiting stable disease. EC1456 at 2 μmol/kg (two times a week for two weeks) produced anti-tumor activity in mice bearing LU1147 tumors with 5 animals exhibiting stable disease. EC1456 at 4 μmol/kg (once a week for two weeks) produced anti-tumor activity in mice bearing LU1147 tumors with 6 animals exhibiting stable disease. See FIG. 3.

c. Antitumor Activity in Large KB Tumor Model

Female Balb/c nu/nu mice were fed ad libitum with folate-deficient chow (Harlan diet #TD01013) for the duration of the experiment. KB tumor cells were inoculated subcutaneously at the right flank of each mouse. Mice were dosed with EC1456 at 2 μmol/kg, TIW×2 after the tumors reached an average of 700, 1000, and 1400 mm³ through the lateral tail vein under sterile conditions in a volume of 200 μL of phosphate-buffered saline (PBS).

Growth of each s.c. tumor was followed by measuring the tumor two times per week. Tumors were measured in two perpendicular directions using Vernier calipers, and their volumes were calculated as 0.5×L×W², where L=measurement of longest axis in mm and W=measurement of axis perpendicular to L in mm

### Results:

EC1456 at 2 μmol/kg (three times a week for two weeks) produced excellent anti-tumor activity with 100% cures in both the 1000 and 1400 mm³ groups. See FIG. 4.

d. Antitumor Activity in Endometrial, Triple Negative Breast and Ovarian Tumor Models

Female Balb/c nu/nu mice were fed ad libitum with folate-deficient chow (Harlan diet #TD01013) for the duration of the experiment. Primary human Endometrial model ST040, TNBC models ST502 and ST738 and Ovarian model ST024 fragments (2-4 mm in diameter) were inoculated subcutaneously at the right flank of each mouse. Mice were randomized into 6 experimental groups of 5 or 3 mice each and test articles were injected through the lateral tail vein under sterile conditions in a volume of 200 μL of phosphate-buffered saline (PBS). These materials were obtained from and studies were performed at South Texas Accelerated Research Therapeutics, 4383 Medical Drive, San Antonio, Tex. 78229

Growth of each s.c. tumor was followed by measuring the tumor two times per week until a volume of 1200 mm³ was reached. Tumors were measured in two perpendicular directions using Vernier calipers, and their volumes were calculated as 0.5×L×W², where L=measurement of longest axis in mm and W=measurement of axis perpendicular to L in mm

### Results:

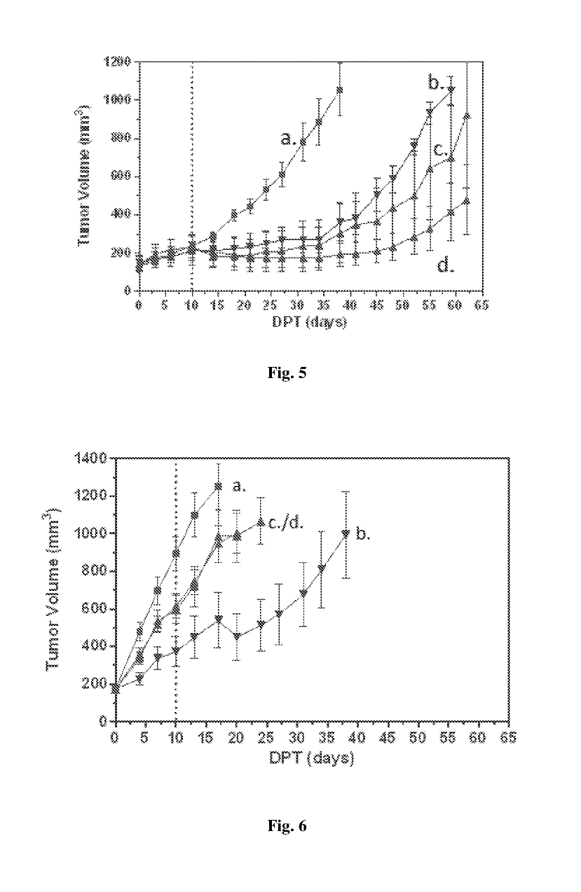

i. Endometrial ST040 Model: Treatment with 15 mg/kg of Paclitaxel (once a week for two weeks) produced minimal anti-tumor activity with zero animals exhibiting stable disease. EC1456 at 1.5 μmol/kg (two times a week for two weeks) and 3 μmol/kg (once a week for two weeks) produced slightly better anti-tumor activity with 2 animals exhibiting stable disease/1 animal exhibiting PR and 2 animals exhibiting stable disease respectively. See FIG. 5.

ii. Triple Negative Breast Cancer (TNBC) ST502 Model: Treatment with 1 mg/kg of Eribulin mesylate (once a week for two weeks) produced minimal anti-tumor activity with 1 animal exhibiting stable disease/1 animal exhibiting PR. EC1456 at 2 μmol/kg (two times a week for two weeks) and 4 μmol/kg (once a week for two weeks) produced no anti-tumor activity. See FIG. 6.

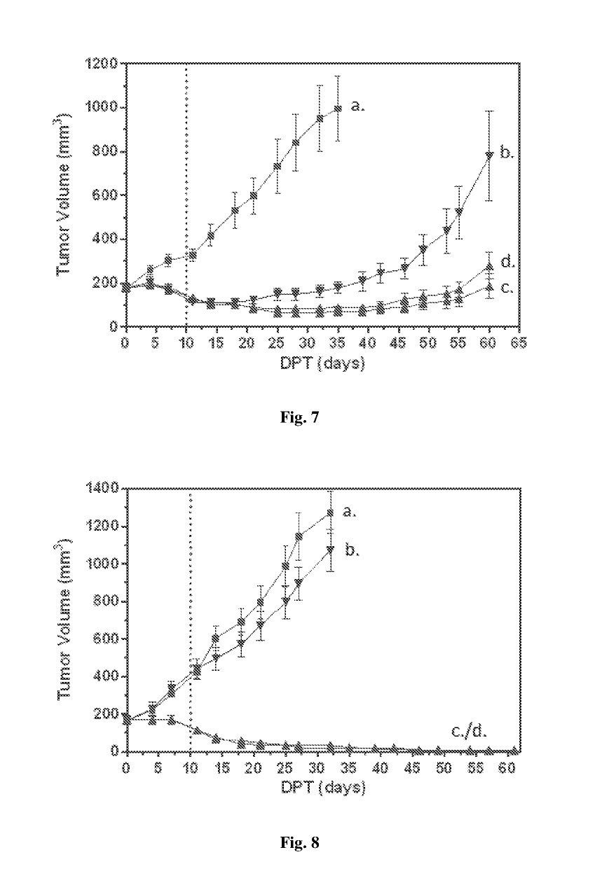

iii. Triple Negative Breast Cancer (TNBC) ST738 Model: Treatment with 1 mg/kg of Eribulin mesylate (once a week for two weeks) produced some anti-tumor activity with 5 animals exhibiting stable disease/2 PR's. EC1456 at 2 μmol/kg (two times a week for two weeks) and 4 μmol/kg (once a week for two weeks) also produced some anti-tumor activity with 2 animals exhibiting stable disease/3 animals exhibiting PR's and 2 animals exhibiting stable disease/5 animals exhibiting PR's. See FIG. 7.

iv. Ovarian ST024 Model: Treatment with 15 mg/kg of Paclitaxel (once a week for two weeks) produced no anti-tumor activity. EC1456 at 2 μmol/kg (two times a week for two weeks) and 4 μmol/kg (once a week for two weeks) produced curative (100% animals exhibiting cures) anti-tumor activity. See FIG. 8.

### 3. Clinical Studies:

Study Design: This is a Phase 1, multicenter, open-label, non-randomized, dose-escalation oncology study to evaluate the administration of Compound I in two schedules: Schedule #1: BIW on Weeks 1 and 2 of a 3-week schedule and Schedule #2: once weekly on Weeks 1 and 2 of a 3-week schedule.

### Study Population:

I. Inclusion Criteria

To qualify for enrollment, the following criteria must be met:

1. Patients must have the ability to understand and sign an approved informed consent form (ICF).

2. Patients must be≧18 years of age.

3. Patients must have histology confirmed metastatic or locally advanced solid tumor (preferably TNBC, NSCLC, ovarian, endometrial) that has failed to respond to standard therapy, is not a candidate for standard therapy, or for which standard therapy does not exist.

4. Patients must have an Eastern Cooperative Oncology Group (ECOG) performance status of 0 or 1.

5. Patients must have at least one measurable lesion per RECIST v1.1 criteria.

6. For the purpose of obtaining a RECIST v1.1 baseline scan, patients must have a radiological evaluation conducted no more than 28 days prior to beginning study therapy. Note: For patients with a history of CNS metastasis, baseline radiological imaging must include evaluation of the brain (MRI preferred or CT with contrast).

7. Patients must have recovered (to baseline/stabilization) from prior cytotoxic-therapy-associated acute toxicities.

8. Patients with prior radiation therapy are eligible if they meet the following criteria:
a) Previous radiation therapy is allowed to<25% of the bone marrow (Cristy and Eckerman, 1987).
b) Prior radiotherapy must be completed at least 2 weeks before patient begins study therapy.
c) Patient must have recovered from the acute toxic effects of the treatment before beginning study therapy.
d) Palliative for pain or symptom control must be completed at least one week before patient begins study therapy, and these lesions must be excluded as target and non-target lesions.

9. Patients must have adequate organ function:
a) Bone marrow reserve: Absolute neutrophil count (ANC)≧1.5×10⁹/L. Platelets≧100×10⁹/L. Hemoglobin≧9 g/dL.
b) Cardiac:
   i. Left ventricular ejection fraction (LVEF) equal to or greater than the institutional lower limit of normal. LVEF must be evaluated within 28 days prior to C1D1.
   ii. Cardiac Troponin I within normal limit.
c) Hepatic: Total bilirubin ≦1.5 x the upper limit of normal (ULN). Alanine aminotransferase (ALT), aspartate aminotransferase (AST)≦3.0×ULN OR <5.0×ULN for patients with liver metastases.
d) Renal: Serum creatinine≦1.5×ULN, or for patients with serum creatinine>1.5 ULN, creatinine clearance≧50 mL/min.

10. Patients of childbearing potential:
a) All women of childbearing potential MUST have a negative serum pregnancy test within 1 week prior to the ^{99m}Tc-etarfolatide imaging procedure and within 1 week prior to treatment with Compound I.
b) Women of child bearing potential must practice an effective method of birth control (e.g., oral, transdermal or injectable contraceptives, intrauterine device [IUD], or double-barrier contraception, such as diaphragm and spermicidal jelly) for the duration of their participation in the trial through 90 days following the last dose of Compound I.
c) Male patients who are sexually active must practice an effective method of birth control (e.g., condom and spermicidal jelly). Effective birth control methods should be used throughout study participation and for at least 90 days following the last dose of Compound I.

II. Exclusion Criteria

The presence of any of the following will exclude patients from the study:

1. More than 4 prior cytotoxic/biologics regimens for metastatic disease. Neoadjuvant and adjuvant treatments would not count towards this criterion (Note: hormonal therapy also would not count towards this criterion).

2. Chemotherapy, immunotherapy or biological therapy (including monoclonal antibodies) within 28 days prior to Compound I administration.

3. Known hypersensitivity to the components of the study therapy or its analogs.

4. Carcinomatous meningitis and/or symptomatic central nervous system (CNS) metastases. Note: Asymptomatic patients with stable CNS metastatic lesions in CT or MRI scans in the last 6 months are eligible.

5. Malignancies that are expected to alter life expectancy or may interfere with disease assessment. Patients with adequately treated non-melanoma skin cancer, carcinoma in situ of the cervix, or low-grade (Gleason score≦6) localized prostate cancer, ductal carcinoma in situ (DCIS) and patients with prior history of malignancy who have been disease free for more than 3 years are eligible.

6. Serious cardiac illness or medical conditions such as unstable angina, pulmonary embolism, or uncontrolled hypertension.

7. Anti-folate therapy such as methotrexate for rheumatoid arthritis.

8. Pregnant or lactating women.

9. Other concurrent chemotherapy, immunotherapy, radiotherapy, or investigational therapy.

10. Active infections (e.g., hepatitis or HIV carriers)

### Treatments & Regimens:

I. Folate Receptor Status Determination (^{99m}Tc-etarfolatide Administration); Prior to the ^{99m}Tc-etarfolatide imaging procedure, patients received one intravenous (IV) injection of 0.5 mg of folic acid, followed within 1-3 minutes by an injection of 0.1 mg of etarfolatide labeled with 20-25 mCi of technetium-99m. Patients then underwent SPECT imaging of the regions(s) known to contain the target lesion(s) approximately 1 hour after injection of ^{99m}Tc-etarfolatide. Folate receptor status was determined by visual inspection of images.

II. Compound I Administration: Compound I was administered as an intravenous bolus injection in two different schedules: Schedule #1: biweekly on Weeks 1 and 2, i.e. on Days 1, 4, 8, 11, of a 3-week cycle or 4-week cycle, and Schedule #2: once weekly on Weeks 1 and 2, i.e. Days 1 and 8 of a 3-week cycle. Treatment was allowed to continue until the patient experienced progressive disease (PD) or intolerable toxicity. Patients discontinued multivitamins or supplements containing folic acid for the duration of the Compound I treatment period.

II. Schedule #1 BIW Dosing Dose Finding: Cohorts consisting of 3-6 patients per dose level were treated with Compound I following a 3+3 schema. DLTs observed in Cycle 1 were used to determine whether additional patients should be enrolled at the same dose level, at a lower dose level, or a higher dose level according to the rules outlined below.
If 0/3 or 1/6 patients experience a DLT; the dose level will be escalated.
If 1/3 experience a DLT; another three patients will be evaluated at this dose level.
If 2/6 patients experience a DLT the dose escalation stage of the trial will be terminated, and the dose directly below the current dose will be considered the MTD.
If ≧2/3 or ≧3/6 patients experience a DLT; the dose directly below the current dose will be explored to 6 patients. If there are <2/6 DLT in that dose level, it will be considered as the MTD.

Intermediate, not previously studied dose levels may be explored, if evaluation of toxicity at such a dose is desired. Decisions for new dose levels will be made based on available preclinical and/or clinical data, and will be made jointly by the Investigator and study sponsor.

II. Schedule #1 BIW Dosing Dose Escalation Scheme: All patients in a dose cohort completed the Cycle 1 DLT (Dose Limiting Toxicity) evaluation before dosing was initiated at the next higher dose level. Only toxicities occurring during the first cycle of study therapy were considered as DLTs and utilized to inform dose escalation decisions.

The table below outlines the dose escalation scheme for Compound I for Schedule #1 BIW dosing, with eight doses levels of Compound I planned. Should the MTD not be determined after escalation of Compound Ito dose level 8, Compound I may continue to be dose escalated in 25% increments.

Schedule #1 Dose Escalation Scheme for Compound I

| Level | Dose (mg/m²) |
|---|---|
| 1 | 0.5 |
| 2 | 1.0 |
| 3 | 1.5 |
| 4 | 2.0 |
| 5 | 2.5 |
| 6 | 3.5 |
| 7 | 4.5 |
| 8 | 6.0 |

NOTE: Although a patient's actual BSA may exceed 2.0 m², the maximum allowable dose of Compound I must be calculated using a BSA that does not exceed 2.0 m².

III. Schedule #2 Once Weekly Dosing Dose Escalation Scheme: The continuous reassessment model (CRM) will be used to determine the MTD for this dose schedule.

All patients must complete the Cycle 1 DLT evaluation before dosing is initiated at the next higher dose level. Only toxicities occurring during the first cycle of study therapy will be considered as DLTs and utilized to inform dose escalation decisions.

The MTD is defined as the dose, at which the probability of a dose limiting toxicity (DLT) is equal to τ=20%.

This dose escalation is designed by using the likelihood-based version of the Continual

Reassessment Method (CRM; O'Quigley & Shen, 1996).

In order to apply the CRM, the dose escalation scheme is split into two stages: the initial dose escalation stage and the model guided stage (Paoletti et al., 2006).

In the initial dose escalation stage, patients are assigned to doses one at a time. The starting dose is 1.5 mg/m². The next doses are 2.0, 2.5, 3.5, 4.5, and 6.0 mg/m². The doses are escalated until the first occurrence of DLT is observed. At that point, the model guided stage is initiated.

In case DLT is observed at the lowest dose in the initial doseescalation stage, the investigators, in collaboration with study sponsor, will re-consider the dose range to be used in the trial.

In case the highest dose is reached without DLT, patients are continued to be assigned to this dose until the maximum of 10 patients, unless a DLT occurs. The probability of observing no

DLT among 10 patients is at most 10.7% when the true probability of DLT is equal to 20% or more.

In the model guided stage, the assignment of the doses to the cohorts of three patients is based on the value of the probability of DLT estimated from a dose toxicity model. In particular, the following model is assumed:

π(dᵢ; β)=xᵢ^{β}

where β>0, π(dᵢ; β) is the probability of DLT at dose dᵢ, and xᵢ is an appropriate re-coding of the dose, assuming β=1 and a priori expectations of the probability of DLT for the doses considered in the trial (O'Quigley & Shen, 1996).

The parameter β determines the form of the dose toxicity relationship and is used to select the dose for the next patients. In particular, after obtaining the information about DLT for a particular cohort of three patients, the dose toxicity model is fitted to the DLT data for all the patients and the maximum-likelihood estimate of β is computed. The next cohort of three patient is assigned the dose, for which the probability of DLT, estimated by using the updated value of β, is as close as possible to τ.

Note that the decision about the assignment of the model recommended dose is to be confirmed or modified by the investigators in collaboration with study sponsor and the statistician running the dose escalation model.

The model guided stage stops when the maximum sample size of 30 patients has been achieved (including the initial dose-escalation stage). The MTD is defined as the dose, for which the estimated probability of DLT, given the estimate of the parameter β based on all included patients, is the closest to τ.

The following are initial estimates of the probability of DLT at the doses that are used in the trial for Schedule #2 Once Weekly Dosing:

| | | Probability | Re-coded |
|---|---|---|---|
| Level | Dose (mg/m²) | of DLT | dose xᵢ |
| 1 | 1.5 | 1% | .01 |
| 2 | 2.0 | 5% | .05 |
| 3 | 2.5 | 15% | .15 |
| 4 | 3.5 | 25% | .25 |
| 5 | 4.5 | 45% | .45 |
| 6 | 6.0 | 65% | .65 |

Intermediate, not previously studied dose levels may be explored, if evaluation of tolerability at such a dose is desired. Decisions for new dose levels will be made based on available preclinical and/or clinical data, and will be made jointly by the Investigator and study sponsor.

IV: DLT Definition for both schedules:

DLTs will be based on events occurring in Part A during the first cycle of therapy and the adverse events must be drug related (i.e. definitely, probably or possibly):
≧Grade 4 hematological toxicity.
Grade 3 neutropenia with fever >38.5° C. and/or infection requiring antibiotic or anti-fungal treatment.
≧Grade 3 non-hematological toxicity
Grade 3 nausea or vomiting lasting more than 72 hours.
A delay of >2 weeks in the scheduled administration of Compound I due to drug-related toxicity.
Schedule #1: BIW Dosing Schedule: Inability to administer at least 3 of the 4 scheduled doses of Compound I in a cycle due to drug-related toxicity.
Schedule #2: Once Weekly Dosing Schedule: Inability to administer both doses of

Compound I in a cycle due to drug-related toxicity.

In the event that a patient drops out resulting in the lack of information about the DLT within the required observation period, the patient will be replaced by a new one, who will be enrolled at the exact same dose.

V. Route of Administration:

Compound I was administered via IV bolus injection.

VI. Clinical Laboratory Evaluations

During the Compound I treatment phase of the clinical trial, the following procedures should be performed as indicated:
For all schedules: Hematology (as noted above) within 3 days prior to Days 1 and 8 of each cycle, and at follow-up. If the absolute neutrophil count (ANC) is determined to be <1.0×10⁹/L, or the platelet count is <100×10⁹/L, repeat testing should occur at intervals chosen by the clinical investigator in order to assure patient safety and to document duration of nadir (i.e., neutropenia, anemia, etc).
For Schedule #1: BIW dosing schedule: Hematology will also be obtained on Day 15 +/− 1 day of each cycle to capture potential nadir counts of the cycle.
For Schedule #2: once weekly dosing schedule: Hematology will also be obtained on Day 15 +/−1 day of each cycle to capture potential nadir counts of the cycle.
Serum chemistries (as noted above):
   For Schedule #1: BIW dosing schedule: Within 3 days prior to Days 1 and 8, and on Day 15 +/−1 day of each cycle and at follow-up.
   For Schedule #1: BIW dosing schedule: Within 3 days prior to Days 1 and 8, and on Day 15 +/−1 day of each cycle and at follow-up.
For all schedules: Complete urinalysis should occur within 3 days prior to Days 1 and 8 of each cycle and at follow-up.
In addition to the requirements listed above, the principal investigator may conduct additional hematology, serum chemistry and urine analyses (and manage abnormalities identified as part of this additional testing) as medically required.
Troponin-I
   For Schedule #1: BIW Dosing Schedule: on Days 1, 4, 8, and 11 of first 2 cycles.
      Can be drawn up to one day before Day 8 and 11.
      If there was no elevation of Troponin-I in the first two cycles, they will need to be evaluated only on days 1 and 8 in subsequent cycles.
   For Schedule #2: Once Weekly Dosing Schedule: Troponin-I on Days 1, and 8.
      Can be drawn up to one day before Day 8.

### On-treatment Procedures and Assessments:

1. ^{99m}Tc-etarfolatide scan

After screening and registration, all patients received a ^{99m}Tc-etarfolatide scan.

II. Week 1—Treatment
Directed physical exam with weight (for Compound I dose calculation based on BSA—NOTE: Although a patient's actual BSA may exceed 2.0 m², the maximum allowable dose of Compound I must be calculated using a BSA that does not exceed 2.0 m²) and ECOG performance status evaluation within 3 days prior to Days 1.
Sample collection for hematology, serum chemistry, urinalysis prior to Compound I administration within 3 days prior to drug administration on Day 1.
Repeat pregnancy test for all women of child-bearing potential prior to Compound I administration on Day 1 (Cycle 1 only).
Documentation of concomitant medications.
Obtain weight for Compound I dose calculation based on BSA—NOTE: Although a patient's actual BSA may exceed 2.0 m², the maximum allowable dose of Compound I must be calculated using a BSA that does not exceed 2.0 m².
Obtain a pre dose ECG
For Schedule #1: BIW Dosing Schedule: Administer Compound I on Days 1 and 4. For Schedule #2: Once Weekly Dosing Schedule: Administer Compound I on Day 1.
Sample for PK analysis within 15 minutes prior to administration of Compound I and at 2, 5, 10, 20, 30, 45, 60, 90, 150 minutes, and 240 minutes (+/− 30 minutes) post 10 cc saline flush after Compound I dose administration is completed, Day 1 (Cycle 1 only).
Vital signs (BP, PLS, RR) prior to the administration of Compound I and then every 15 (±5) minutes for 30 minutes post administration of Compound I.
Schedule #1: BIW Dosing Schedule: Troponin-I on Days 1 and 4 of first 2 cycles.
   Can be drawn up to one day before Day 1 and 4.
   If there was no elevation of Troponin-I in the first two cycles, it will need to be evaluated only on days 1 and 8 in subsequent cycles.
Schedule #2: Once Weekly Dosing Schedule: Troponin-I on Day 1.
   Can be drawn up to one day before Day 1.
Tumor markers, if clinically indicated.
Monitor AEs and SAEs

III. Week 2—Treatment:
Directed physical exam with weight (for Compound I dose calculation based on BSA—NOTE: Although a patient's actual BSA may exceed 2.0 m², the maximum allowable dose of Compound I must be calculated using a BSA that does not exceed 2.0 m²) and ECOG performance status evaluation within 3 days prior to Day 8.
Sample collection for hematology, serum chemistry, and urinalysis prior to Compound I administration within 3 days prior to drug administration on Day 8.
Documentation of concomitant medications.
Schedule #1: BIW Dosing Schedule: Administer Compound I on Days 8, 11.
Schedule #2: Once Weekly Dosing Schedule: Administer Compound I on Day 8.
Sample for PK analysis within 15 minutes prior to administration of Compound I and at 2, 5, 10, 20, 30, 45, 60, 90, 150 minutes, and 240 minutes (+/− 30 minutes) post 10 cc saline flush after Compound I dose administration is completed, Day 11 (Cycle 1 only) for Schedule #1: BIW Dosing Schedule and Day 8 (Cycle 1 only) for Schedule #2: Once Weekly Dosing Schedule.
Vital signs (BP, PLS, RR) prior to the administration of Compound I and then every 15 (+5) minutes for 30 minutes post administration of Compound I.
Schedule #1: BIW Dosing Schedule: Troponin-I on Days 8 and 11 of first 2 cycles.
   Can be drawn up to one day before Day 8 and 11.
   If there was no elevation of Troponin-I in the first two cycles, it will need to be evaluated only on days 1 and 8 in subsequent cycles.
Schedule #2: Once Weekly Dosing Schedule: Troponin-I on Day 1.
   Can be drawn up to one day before Day 1.
Tumor markers, if clinically indicated.
Monitor AEs and SAEs

IV. Week 3—Rest/Observation:
Sample collection for hematology on Day 15 +/− 1 day.
Sample collection for chemistry on Day 15 +/− 1 day.
Tumor markers, if clinically indicated.
Monitor Adverse Events and SAEs (phone evaluation is adequate)
Documentation of concomitant medications (phone evaluation is adequate)

V. Radiological Assessments:

CT, MRI, PET scan or chest x-ray performed for lesion assessment per RECIST V1.1 criteria after every 2 cycles of therapy (±3 days). The historical or screening imaging used to define the target lesions should be the modality used for all subsequent follow-up target lesion assessments. If a patient's scan after 2 cycles exhibits evidence of tumor response (CR or PR), a “confirmatory” scan may be conducted no less than 4 weeks from when the response was observed. The next regularly-scheduled radiographic evaluation can act as the “confirmatory” scan. If a patient has experienced tolerable toxicities and has radiographic evidence of tumor size stabilization or regression at the time of radiological assessment, or if tumor markers are indicative [in the investigator's opinion] of clinical benefit, that patient is eligible to continue Compound I treatment for a duration deemed clinically appropriate by the investigator.

MUGA scan will be performed after every 2 cycles of therapy (+/− 3 days) to assess for cardiac function.

### Post-Therapy (Follow-up) Procedures and Assessments:

Patients returned to the investigative site for a follow-up visit approximately 30 days after the last dose of study drug. The following evaluations were conducted:
Focused history and physical exam (including weight and vital signs) conducted to assess general status
Monitor Adverse Events and Serious Adverse Events (SAEs)
Documentation of concomitant medications
ECOG status evaluation
Serum chemistries, hematology, urinalysis, and troponin (as per section 9.2.2)
Tumor markers, if clinically indicated
For patients who progress with PD, an (Optional) Repeat ^{99m}Tc-etarfolatide imaging. A phone contact no less than 4 days following the repeat ^{99m}Tc-etarfolatide imaging is required to monitor AEs and SAEs.

In the event a patient could not return to the investigative site for the follow-up assessment, the patient was contacted and the following information obtained:
Monitor Adverse Events and Serious Adverse Events (SAEs)
Documentation of concomitant medications
Patient status

### Results:

Three patients enrolled into DL1 and DL2 respectively. All 6 patients were eligible for DLT, safety, and anti-tumor assessment. In DL1, 3 patients received a median of 8 cycles of study drug (range: 2-9). In DL2, 3 patients have received a median of 1 cycle (range: 1-2) in

DL1. There have been no dose delays, omissions, or reductions due to toxicity in either dose level. No deaths occurred on treatment or within 30 days of study discontinuation, treatment related serious adverse events (SAEs) or DLTs.

In DL1, there was one SAE (Grade 3 vomiting in cycle 6), attributed to a viral syndrome. There were no SAEs in DL2. No Grade 4 or treatment-related Grade 3 TEAEs were observed in

DL1. Regardless of the relatedness to study drugs, there were no >Grade 3 TEAEs observed in DL2. To date, there has been no evidence of cumulative or late-emerging treatment-related AEs in either dose level. In DL1, TEAEs such as tinnitus, constipation, asthenia, ataxia, dizziness, and orthostatic hypotension, have been mild and self-limited. TEAEs such as abdominal pain, diarrhea, vomiting, hypoglycemia and pain have been mild and self-limited in DL2.

Durable stable disease with EC1456 has been observed in the BIW dosing schedule in 3 folate receptor evaluable patients (1 patient with gastroesophageal cancer, 1 with pleural mesothelioma, and 1 patient with small cell lung cancer). Durable stable disease with EC1456 has been observed in the QW dosing schedule in 1 folate receptor evaluable patient (non-small cell lung cancer) and 1 folate receptor non-evaluable patient (Leiomyosarcoma). Results are summarized in Table I. The shorthand identifier FR (%) stands for “folate receptor (percentage of evaluable tumors in the patient that are folate receptor positive)”.

TABLE 1

| | Folate | Prior | | | | |
|---|---|---|---|---|---|---|
| | Receptor | Systemic | | | Cycles | |
| Study Disease | Status | Treatments | Dose | Schedule | Cpd 1 | Response |
| Adenocarcinoma | FR (100%) | Palifosfamide; | 0.5 mg/m² | BIW | >14 | Stable |
| of | | 5-FU | | | | disease |
| gastresophogeal | | Capecitabine | | | | |
| junction | | | | | | |
| Pleural | FR (100%) | Pemetrexed + | 0.5 mg/m² | BIW | >14 | Stable |
| mesothelioma | | Cisplatin; | | | | disease |
| | | Investigational | | | | |
| | | Agent; | | | | |
| | | Pemetrexed + | | | | |
| | | Carboplatin | | | | |
| Triple-negative | FR (20-80%) | Gemcitabine + | 0.5 mg/m² | BIW | 2 | Progressive |
| breast cancer | | Paclitaxel; | | | | disease |
| | | Capecitabine; | | | | |
| | | Vinorelbine; | | | | |
| | | Eribulin | | | | |
| Non-small cell | FR (0%) | Pemetrexed + | 1.0 mg/m² | BIW | 2 | Progressive |
| lung cancer | | Carboplatin; | | | | disease |
| | | Docetaxel | | | | |
| Triple-negative | FR (100%) | Paclitaxel; | 1.0 mg/m² | BIW | 1 | Progressive |
| breast cancer | | Cyclophosphamide + | | | | disease |
| | | Docetaxel; | | | | |
| | | Cyclophosphamide + | | | | |
| | | Doxorubicin | | | | |
| Endometrial | FR (100%) | Gemcitabine + | 1.0 mg/m² | BIW | 1 | Progressive |
| cancer | | Paclitaxel + | | | | disease |
| | | Carboplatin; | | | | |
| | | Gemcitabine + | | | | |
| | | Carboplatin; | | | | |
| | | Paclitaxel; | | | | |
| | | Regorafinib | | | | |
| Leiomyosarcoma | Not | Gemcitabine + | 1.5 mg/m² | QW | 12 | Stable |
| | evaluable | Docetaxel | | | | disease |
| Small cell lung | FR (20-80%) | Gemcitabine + | 2.0 mg/m² | BIW | 6 | Stable |
| cancer | | Etoposide | | | | disease |
| Non-small cell | FR (100%) | Erlotinib | 2.5 mg/m² | QW | 8 | Stable |
| lung cancer | | Carboplatin + | | | | disease |
| | | Paclitaxel + | | | | |
| | | Bevacuzimab | | | | |
| | | Premexetred | | | | |

What is claimed is:

1. A method for treating a cancer in a patient in need of such treatment comprising, administering to the patient a therapeutically effective amount of a compound of the formula I 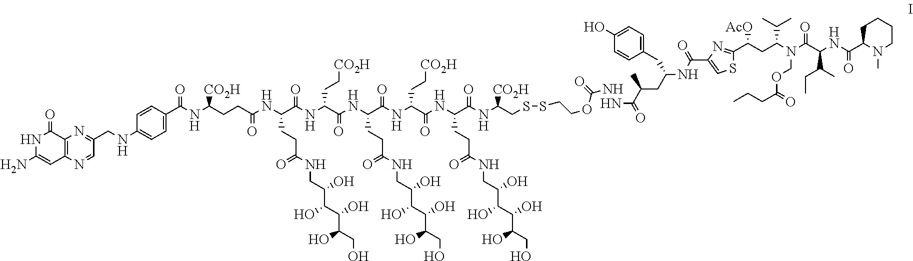 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c2ccc(NCc3cnc4cc(N)[nH]c(=O)c4n3)cc2)C(=O)O)C(=O)O)cs1)C(C)C]
or a pharmaceutically acceptable salt thereof, wherein the cancer is selected from the group consisting of non-small cell lung cancer, ovarian cancer, pleural mesothelioma, adenocarcinoma of gastroesophogeal junction, leiomyosarcoma and small cell lung cancer.

2. The method of claim 1, wherein the cancer is a folate receptor expressing cancer.

3. The method of claim 1, wherein the compound is at least about 98 percent pure.

4. The method of claim 1, wherein the compound of formula I, or a pharmaceutically acceptable salt thereof, is administered in a parenteral dosage form.

5. The method of claim 4, wherein the parenteral dosage form is selected from the group consisting of intradermal, subcutaneous, intramuscular, intraperitoneal, intravenous, and intrathecal.

6. The method of claim 1, wherein the therapeutically effective amount is from about 0.5 mg/m² to about 20.0 mg/m².

7. The method of claim 1, further comprising detecting folate receptor overexpression by the cancer.

8. The method of claim 7, wherein the step of detecting occurs before the step of administering.

9. The method of claim 8, wherein the detecting is performed by imaging and wherein the imaging is selected from the group consisting of SPECT imaging, PET imaging, IHC, and FISH.

10. The method of claim 9, wherein the detecting is performed by SPECT imaging.

11. The method of claim 1, further comprising determining the folate receptor status of the patient by imaging.

12. The method of claim 11, wherein the imaging is SPECT imaging.

13. The method of claim 12, wherein the folate receptor status is based on a measurement of the percentage of evaluable lesions in the patient that are folate receptor positive.

14. The method of claim 11, wherein the folate receptor status of the patient correlates with a clinical benefit to the patient.

15. The method of claim 14, wherein the clinical benefit is selected from the group consisting of inhibition of tumor growth, stable disease, a partial response, and a complete response.

16. The method of claim 13, wherein the folate receptor positive lesions indicate functionally active folate receptors.

17. The method of claim 8, wherein the step of detecting comprises administering a conjugate of the formula III 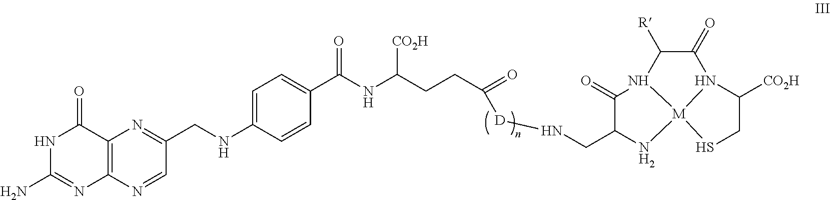
or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, wherein D is a divalent linker, wherein n is 0 or 1, and wherein M is a cation of a radionuclide.

18. The method of claim 17, wherein M in the conjugate, or a pharmaceutically acceptable salt thereof, is selected from the group consisting of an isotope of gallium, an isotope of indium, an isotope of copper, an isotope of technetium, and an isotope of rhenium.

19. The method of claim 17, wherein M in the conjugate, or a pharmaceutically acceptable salt thereof, is an isotope of technetium.

20. The method of claim 17, wherein the conjugate is of the formula 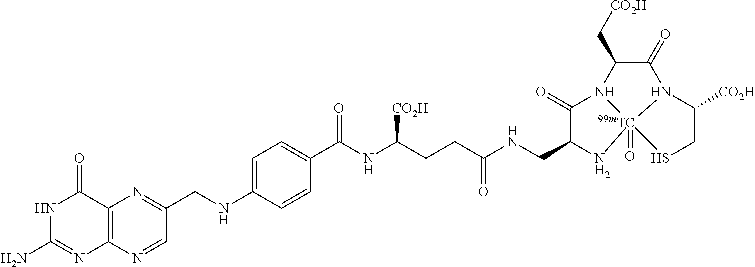
or a pharmaceutically acceptable salt thereof.
